(19) United States
(12) Patent Application Publication
(10) Pub. No.: US 2025/0332265 A1
(43) Pub. Date: Oct. 30, 2025

(54) PSMA TARGETED CONJUGATE COMPOUNDS AND USES THEREOF

(71) Applicant: CASE WESTERN RESERVE UNIVERSITY, Cleveland, OH (US)

(72) Inventors: James Basilion, Cleveland, OH (US); Xinning Wang, Cleveland, OH (US); Zhenghong Lee, Cleveland, OH (US)

(21) Appl. No.: 18/708,899

(22) PCT Filed: Nov. 9, 2022
(86) PCT No.: PCT/US2022/079566

§ 371 Date: May 9, 2024

Related U.S. Application Data
(60) Provisional application No. 63/277,426, filed on Nov. 9, 2021.
(60) Provisional application No. 63/348,544, filed on Jun. 3, 2022.
(60) Provisional application No. 63/359,257, filed on Jul. 8, 2022.

(51) Int. Cl. A61K 47/54; A61K 49/00; A61K 51/04; A61P 35/00

(52) U.S. Cl. CPC A61K 47/542; A61K 47/545; A61K 49/0002; A61K 51/0402; A61K 51/0497; A61P 35/00

(57) ABSTRACT

PSMA targeted conjugate compounds, pharmaceutical compositions comprising these compounds, methods for treating and detecting cancers in a subject, methods for identifying cancer cells in a sample are described herein.

### RELATED APPLICATION

This application claims priority from U.S. Provisional Application Nos. 63/277,426, filed Nov. 9, 2021 and 63/348,544 filed Jun. 3, 2022, and 63/359,257, filed Jul. 8, 2022, the subject matter of which are incorporated herein by reference in their entirety.

### TECHNICAL FIELD

This application relates to prostate-specific membrane antigen (PSMA) targeted conjugate compounds and to their use in compositions for targeting, imaging, and treating cancer.

### BACKGROUND

Prostate-specific membrane antigen (PSMA) is a 120 kDa protein expressed in prostate tissues and was originally identified by reactivity with a monoclonal antibody designated 7E11-C5 (Horoszewicz et al., 1987, Anticancer Res. 7:927-935; U.S. Pat. No. 5,162,504). PSMA is characterized as a type II transmembrane protein sharing sequence identity with the transferrin receptor (Israeli et al., 1994, Cancer Res. 54:1807-1811). PSMA is a glutamate carboxy-peptidase that cleaves terminal carboxy glutamates from both the neuronal dipeptide N-acetylaspartylglutamate (NAAG) and gamma-linked folate polyglutamate. That is, expression of PSMA cDNA confers the activity of N-acetylated α-linked acidic dipeptidase or “NAALADase” activity (Carter et al., 1996, PNAS 93:749-753).

PSMA is expressed in increased amounts in prostate cancer, and elevated levels of PSMA are detectable in the sera of these patients (Horoszewicz et al., 1987, supra; Rochon et al., 1994, Prostate 25:219-223; Murphy et al., 1995, Prostate 26:164-168; and Murphy et al., 1995, Anticancer Res. 15:1473-1479). As a prostate carcinoma marker, PSMA is believed to serve as a target for imaging and cytotoxic treatment modalities for prostate cancer. Prostate carcinogenesis, for example, is associated with an elevation in PSMA abundance and enzymatic activity of PSMA. PSMA antibodies, particularly indium-Ill labeled and tritium labeled PSMA antibodies, have been described and examined clinically for the diagnosis and treatment of prostate cancer. PSMA is expressed in prostatic ductal epithelium and is present in seminal plasma, prostatic fluid and urine.

Recent evidence suggests that PSMA is also expressed in tumor associated neovasculature of a wide spectrum of malignant neoplasms including conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma. (Chang et al. (1999) Cancer Res. 59, 3192-3198).

In addition to prostate cancer and other proliferating or neoplastic cells, normal tissues can also express PSMA or PSMA-like molecules with the highest density of non-cancer tissue expression in the kidneys, lacrimal glands, and salivary glands. These tissue represent areas of interference (for PSMA-expressing cancer imaging) or dose-limiting sites of toxicity (for PSMA-targeted cancer therapies).

### SUMMARY

Embodiments described herein relate to PSMA targeted conjugate compounds, pharmaceutical compositions comprising these compounds, methods for treating and detecting cancers (e.g., prostate cancer) in a subject using these PSMA targeted conjugate compounds, and methods for identifying cancer cells (e.g., prostate cancer cells) in a sample using these compounds. Advantageously, the PSMA targeted conjugate compounds after systemic administration to a subject in need thereof show minimal accumulation and/or uptake in non-PSMA targets in non-cancer tissue, such as salivary glands, lacrimal glands, and kidney of the subject.

In some embodiments, the compound can include the general formula (I):

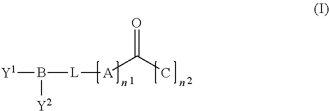

wherein:
n¹ and n² are each independently 1, 2, 3, or 4;
A, B, and C each independently include at least one of a negatively charged amino acid, an acidic amino acid, a non-standard negatively charged or acidic amino acid thereof, a derivative thereof, or an analogue thereof;
L is an optionally substituted aliphatic or heteroaliphatic linking group; and
Y¹ and Y² are each independently a H of B or can include at least one detectable moiety, therapeutic agent, or theranostic agent that is directly or indirectly linked to B, wherein at least one of Y¹ or Y² is not H.

In some embodiments, Y¹ and Y² are not H.

In some embodiments, the compound of formula (I) does not bind to aquaporin (e.g., AQP3), such as aquaporin expressed in the salivary glands, upon administration of the compound to a subject with cancer.

In other embodiments, the compound has a selectivity for PSMA expressing cancer tissue versus non-PSMA expressing non-cancer tissue ≥5 times, ≥10 times, ≥20 times, ≥30 times, ≥40 times, ≥50 times or more times.

In other embodiments, the compound upon administration to a subject in need thereof shows minimal accumulation or uptake in salivary glands of the subject.

In other embodiments, Y¹ and Y² each independently can include at least one radiolabel or radioisotope, radiosensitizer, chelating agent, photosensitizer, fluorescent labeling agent, magnetic resonance imaging (MRI) agent, or anti-cancer agent.

In some embodiments, the anticancer agent is linked to B via a protease cleavable or acid-labile linker. The linker can include a maleimido-caproyl linker-valine-citrulline cleavable peptide-p-aminobenzyl carbamate spacer (MC-VC-PABC) protease cleavable linker.

In some embodiments, at least one of Y¹ or Y² includes a chelating agent optionally complexed with a metal or metal ion. In some embodiments, the chelating agent includes at least one of diethylenetriaminepentaacetate (DTPA), 1,4,7,10-tetraazadodecanetetraacetate (DOTA), 1,4,7,10-tetraazadodecane-1,4,7-triacetate (DO3A), ethylenediaminetetraacetate (EDTA), 1,4,7,10-tetraazacyclotridecanetetraacetic acid (TRITA), 1,4,8,11-tetraazacyclotetradecane-1,4,8,11-tetraacetic acid (TETA), 1,4,7,10-tetraazadodecanetetramethylacetate (DOTMA), 1,4,7,10-tetraazadodecane-1,4,7-trimethylacetate (DO3MA), N,N′,N″,N′″-tetraphosphonatomethyl-1,4,7,10-tetraazacyclododecane (DOTP), 1,4,7,10-tetraazacyclododecane-1,4,7,10-tetrakis(methylene methylphosphonic acid) (DOTMP), 1,4,7,10-tetraazacyclododecane-1,4,7,10-tetrakis(methylene phenylphosphonic acid) (DOTPP), N,N′-ethylenedi-L-cysteine, 1,4,7-triazacyclononane-1,4,7-triacetic acid (NOTA), 1,4,7-triazacyclononane (TACN), N,N′-Bis(2-hydroxy-5-(ethylene-beta-carboxy)benzyl)ethylenediamine N,N′-diacetic acid (HBED-CC), S-2-(4-isothiocyanatobenzyl)-1,4,7,10-tetraazacylododecane tetracetic acid (p-SCN-Bn-DOTA), MeO-DOTA-NCS, [(R)-2-Amino-3-(4-isothiocyanatophenyl)propyl]-trans-(S,S)-cyclohexane-1,2-diamine-pentaacetic acid (CHX-A″-DTPA-NCS), 2-[4-nitrobenzyl]-1,4,7,10,13-pentaazacyclopentadecane-N,N′,N″,N′″,N″″-pentaacetic acid (PEPA), 1,4,7,10,13,16-hexaazacyclooctadecane-N,N′,N″,N′″,N″″-hexaacetic acid (HEHA), desferrioxamine B (DFO), or derivatives thereof.

In some embodiments, the metal ion includes at least one of a radioactive isotope of Ga, I, In, Y, Lu, Bi, Ac, Re, In, Th, Tc, Tl, Tb, Zr, Cu, Rb, At, Pb, Gd, Sm, or Sr. In other embodiments, the metal ion includes at least one of ¹⁸⁶Re, ¹⁸⁸Re, ^{99m}Tc ¹⁵³Gd, ¹¹¹In ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹T, ¹⁶¹T, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th.

In some embodiments, L can include at least one ring selected from the group consisting of an optionally substituted 4 to 7 membered nonaromatic heterocyclic ring and an optionally substituted C4-C7 cycloalkyl ring.

In some embodiments B includes at least one, two, three, four, five, or more negatively charged or acidic amino acids.

In some embodiments, the negatively charged or acidic amino acids can be selected from aspartic acid or aspartate or glutamic acid or glutamate.

In other embodiments, B further includes at least one of lysine, tyrosine, or cysteine.

In other embodiments, the compound can include the general formula (II):

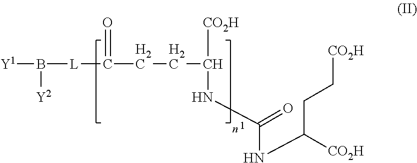

wherein:
n¹ is 1, 2, 3, or 4;
B includes a negatively charged amino acid, an acidic amino acid, a non-standard negatively charged or acidic amino acid thereof, a derivative thereof, or an analogue thereof;
L is an optionally substituted aliphatic or heteroaliphatic linking group; and
Y¹ and Y² are each independently a H of B or can include at least one of a detectable moiety, therapeutic agent, or a theranostic agent that is directly or indirectly linked to B, wherein at least one of Y¹ or Y² is not H.

In some embodiments, Y¹ and Y² are not H.

In some embodiments, Y¹ and Y² each independently include at least one radiolabel or radioisotope, radiosensitizer, chelating agent, photosensitizer, fluorescent labeling agent, magnetic resonance imaging (MRI) agent, or anti-cancer agent.

In some embodiments, B has the following formula:

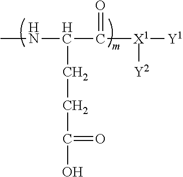

wherein m is 1, 2, 3, or 4, X¹ includes at least one amino acid, and at least one of Y¹ or Y² includes at least one detectable moiety, therapeutic agent, or theranostic agent that is directly or indirectly linked to X¹.

In some embodiments, X¹ further includes at least one of lysine, tyrosine, or cysteine.

In some embodiments, two of Y¹ and Y² include at one radiolabel or radioisotope, radiosensitizer, chelating agent, photosensitizer, fluorescent labeling agent, magnetic resonance imaging (MRI) agent, or anti-cancer agent

In other embodiments, B has the following formula:

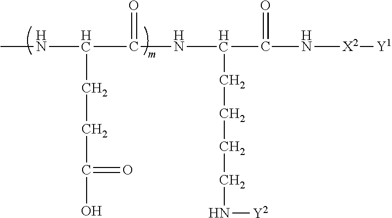

wherein m is 1, 2, 3, or 4;
X² is H or includes at least one amino acid;
Y¹ includes at least one detectable moiety; therapeutic agent, or theranostic agent that is directly or indirectly linked to X² or is absent if X² is H; and
Y² includes at least one of a detectable moiety, therapeutic agent, or theranostic agent.

In other embodiments, one of Y¹ or Y² includes at least one radiolabel or radioisotope, radiosensitizer, chelating agent, photosensitizer, fluorescent labeling agent, magnetic resonance imaging (MRI) agent, or anti-cancer agent.

In some embodiments, the compound can include the general formula:

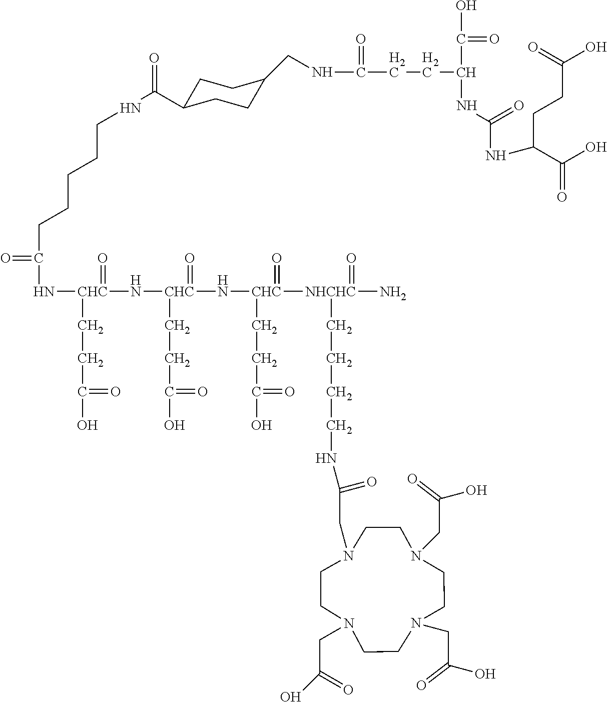

complexed with at least one of a metal contrast agent or metal ion. In some embodiments, the metal ion can include at least one of ¹⁸⁶Re, ¹⁸⁸Re, ^{99m}Tc, ⁵³Gd, ¹¹¹In, ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹Tb, ¹⁶¹Tb, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th or a pharmaceutically acceptable salt thereof.

In some embodiments, the compound includes the following formula:

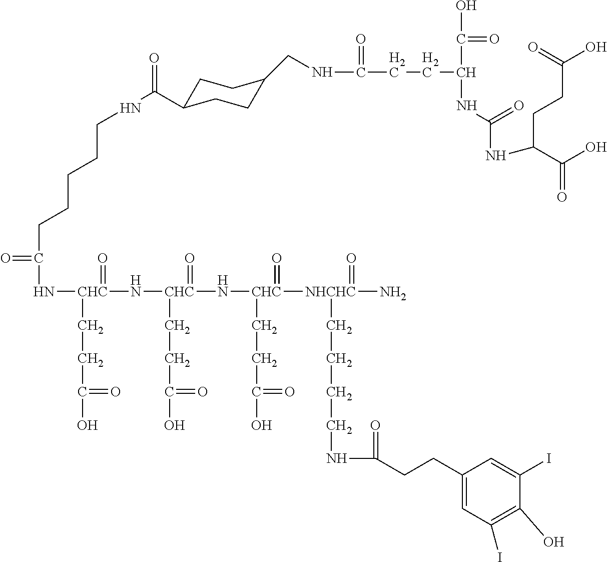

pharmaceutically acceptable salt thereof, wherein the I is ¹²³⁻¹²⁵I or ¹³¹I.

In some embodiments, B has the following formula:

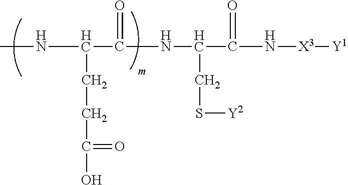

wherein m is 1, 2, 3, or 4;
X³ is H or includes at least one of an amino acid or linker;
Y¹ includes at least one of H, detectable moiety, therapeutic agent, or theranostic agent that is directly or indirectly linked to X³, or is absent if X³ is a H; and
Y² includes at least one of a detectable moiety, therapeutic agent, or theranostic agent.

In other embodiments, Y¹ and Y² include at least one radiolabel or radioisotope, radiosensitizer, chelating agent, photosensitizer, fluorescent labeling agent, magnetic resonance imaging (MRI) agent, or anti-cancer agent.

In some embodiments, the radiosensitizer is a Au ion that is conjugated to a free thiol.

In some embodiments, the compound includes the following formula:

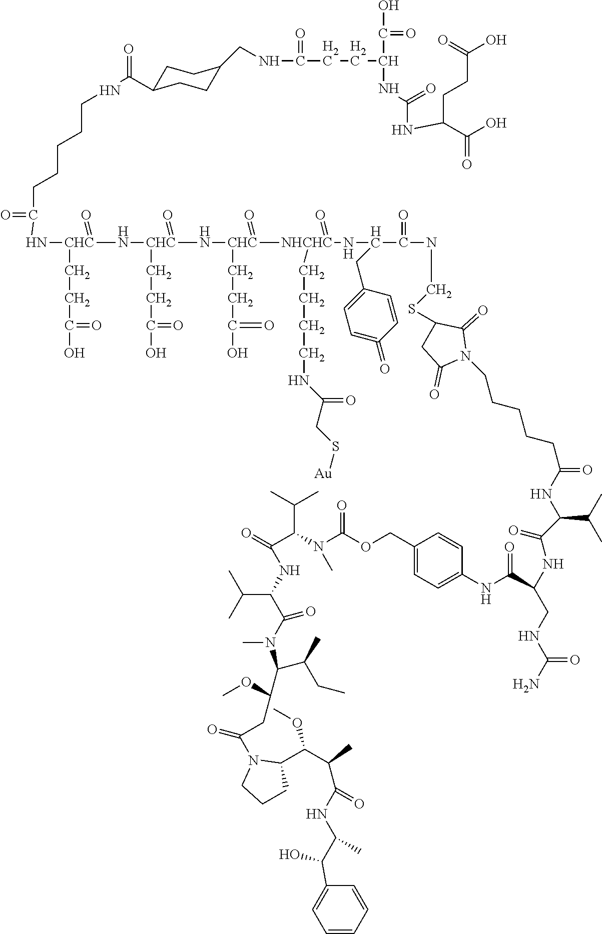

Tn other embodiments. B has the following formula:

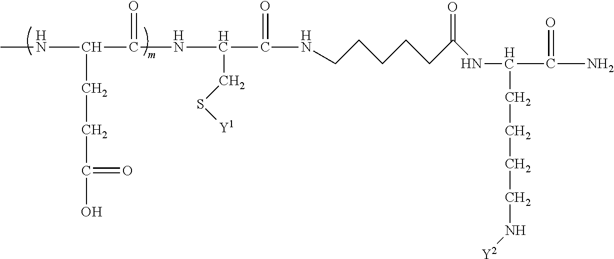

wherein m is 1, 2, 3, or 4; and
Y¹ is a first imaging agent, therapeutic agent, or theranostic agent that is directly or indirectly linked to the thiol group and Y² includes at least one second imaging agent, therapeutic agent, or theranostic agent that is directly or indirectly linked to the amine group.

In some embodiments, each of Y¹ and Y² independently include at least one radiolabel or radioisotope, radiosensitizer, chelating agent, photosensitizer, fluorescent labeling agent, magnetic resonance imaging (MRI) agent, or anti-cancer agent.

In some embodiments, L can include at least one ring selected from the group consisting of an optionally substituted 4 to 7 membered nonaromatic heterocyclic ring and an optionally substituted C4-C7 cycloalkyl ring.

In some embodiments, at least one of Y¹ or Y² includes a chelating agent optionally complexed with a metal contrast agent or metal ion.

In some embodiments, the chelating agent includes at least one of diethylenetriaminepentaacetate (DTPA), 1,4,7,10-tetraazadodecanetetraacetate (DOTA), 1,4,7,10-tetraazadodecane-1,4,7-triacetate (DO3A), ethylenediaminetetraacetate (EDTA), 1,4,7,10-tetraazacyclotridecanetetraacetic acid (TRITA), 1,4,8,11-tetraazacyclotetradecane-1,4,8,11-tetraacetic acid (TETA), 1,4,7,10-tetraazadodecanetetramethylacetate (DOTMA), 1,4,7,10-tetraazadodecane-1,4,7-trimethylacetate (DO3MA), N,N′,N″,N′″-tetraphosphonatomethyl-1,4,7,10-tetraazacyclododecane (DOTP), 1,4,7,10-tetraazacyclododecane-1,4,7,10-tetrakis(methylene methylphosphonic acid) (DOTMP), 1,4,7,10-tetraazacyclododecane-1,4,7,10-tetrakis(methylene phenylphosphonic acid) (DOTPP), N,N′-ethylenedi-L-cysteine, 1,4,7-triazacyclononane-1,4,7-triacetic acid (NOTA), 1,4,7-triazacyclononane (TACN), N,N′-Bis(2-hydroxy-5-(ethylene-beta-carboxy)benzyl)ethylenediamine N,N′-diacetic acid (HBED-CC), S-2-(4-isothiocyanatobenzyl)-1,4,7,10-tetraazacylododecane tetracetic acid (p-SCN-Bn-DOTA), MeO-DOTA-NCS, [(R)-2-Amino-3-(4-isothiocyanatophenyl)propyl]-trans-(S,S)-cyclohexane-1,2-diamine-pentaacetic acid (CHX-A″-DTPA-NCS), 2-[4-nitrobenzyl]-1,4,7,10,13-pentaazacyclopentadecane-N,N′,N″,N′″,N″″-pentaacetic acid (PEPA), 1,4,7,10,13,16-hexaazacyclooctadecane-N,N′,N″,N′″,N″″-hexaacetic acid (HEHA), desferrioxamine B (DFO), or derivatives thereof.

In some embodiments, the metal ion includes at least one of a radioactive isotope of Ga, I, In, Y, Lu, Bi, Ac, Re, In, Th, Tc, Tl, Tb, Zr, Cu, Rb, At, Pb, Gd, Sm, or Sr. In other embodiments, the metal ion includes at least one of ¹⁸⁶Re, ¹⁸⁸Re, ^{99m}Tc ¹⁵³Gd, ¹¹¹In ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹Tb, ¹⁶¹T, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th.

In some embodiments, the compound has the general formula:

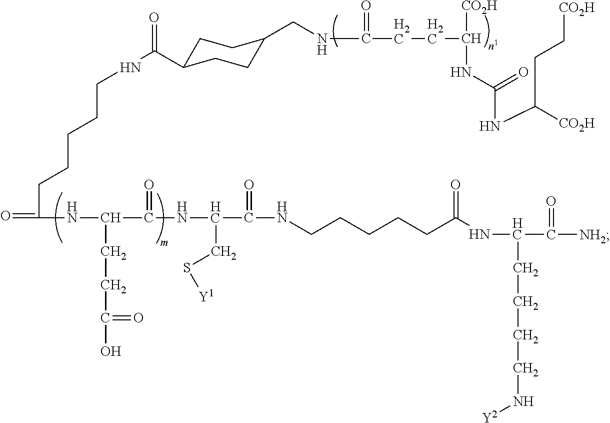

wherein m and n¹ are each independently 1, 2, 3, or 4; and wherein Y¹ includes a radiolabel or radioisotope, radiosensitizer, chelating agent, photosensitizer, fluorescent labeling agent, magnetic resonance imaging (MRI) agent, or anti-cancer agent that is directly or indirectly linked to the thiol group and Y² includes a radiolabel or radioisotope, radiosensitizer, chelating agent, photosensitizer, fluorescent labeling agent, magnetic resonance imaging (MRI) agent, or anti-cancer agent that is directly or indirectly linked to the amine group.

In other embodiments, at least one of Y¹ or Y² is an anti-cancer agent and the other of Y¹ or Y² includes a radiolabel or radionuclide. For example, the anti-cancer agent can be a synthetic anticancer agent, such as monomethyl auristatin E (MMAE), and the radionuclide can include a chelating agent optionally complexed with a metal ion.

In some embodiments, the chelating agent includes at least one of diethylenetriaminepentaacetate (DTPA), 1,4,7,10-tetraazadodecanetetraacetate (DOTA), 1,4,7,10-tetraazadodecane-1,4,7-triacetate (DO3A), ethylenediaminetetraacetate (EDTA), 1,4,7,10-tetraazacyclotridecanetetraacetic acid (TRITA), 1,4,8,11-tetraazacyclotetradecane-1,4,8,11-tetraacetic acid (TETA), 1,4,7,10-tetraazadodecanetetramethylacetate (DOTMA), 1,4,7,10-tetraazadodecane-1,4,7-trimethylacetate (DO3MA), N,N′,N″,N′″-tetraphosphonatomethyl-1,4,7,10-tetraazacyclododecane (DOTP), 1,4,7,10-tetraazacyclododecane-1,4,7,10-tetrakis(methylene methylphosphonic acid) (DOTMP), 1,4,7,10-tetraazacyclododecane-1,4,7,10-tetrakis(methylene phenylphosphonic acid) (DOTPP), N,N′-ethylenedi-L-cysteine, 1,4,7-triazacyclononane-1,4,7-triacetic acid (NOTA), 1,4,7-triazacyclononane (TACN), N,N′-Bis(2-hydroxy-5-(ethylene-beta-carboxy)benzyl)ethylenediamine N,N′-diacetic acid (HBED-CC), S-2-(4-isothiocyanatobenzyl)-1,4,7,10-tetraazacylododecane tetracetic acid (p-SCN-Bn-DOTA), MeO-DOTA-NCS, [(R)-2-Amino-3-(4-isothiocyanatophenyl)propyl]-trans-(S,S)-cyclohexane-1,2-diamine-pentaacetic acid (CHX-A″-DTPA-NCS), 2-[4-nitrobenzyl]-1,4,7,10,13-pentaazacyclopentadecane-N,N′,N″,N′″,N″″-pentaacetic acid (PEPA), 1,4,7,10,13,16-hexaazacyclooctadecane-N,N′,N″,N′″,N″″-hexaacetic acid (HEHA), desferrioxamine B (DFO), or derivatives thereof.

In some embodiments, the metal ion includes at least one of a radioactive isotope of Ga, I, In, Y, Lu, Bi, Ac, Re, In, Th, Tc, Ti, Tb, Zr, Cu, Rb, At, Pb, Gd, Sm, or Sr. In other embodiments, the metal ion includes at least one of ¹⁸⁶Re, ¹⁸⁸Re, ^{99m}Tc ¹⁵³Gd, ¹¹¹In ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹T, ¹⁶¹Tb, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th.

In some embodiments, at least one of Y¹ or Y² is an anti-cancer agent and the other of Y¹ or Y² is a photosensitizer. For example, the anti-cancer agent can be a synthetic anticancer agent, such as monomethyl auristatin E (MMAE), and the photosensitizer can be IR700.

For example, a PSMA targeted conjugate compound that includes MMAE and IR700 can have the following formula:

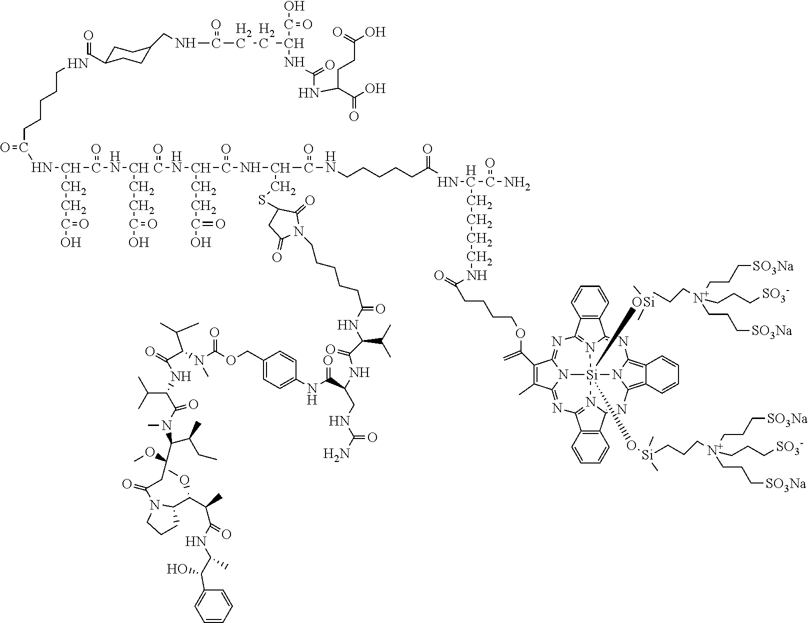

or a pharmaceutically acceptable salt thereof.

In another example, the PSMA targeted conjugate compound includes MMAE and a chelating agent optionally complexed with a metal ion having the following formula:

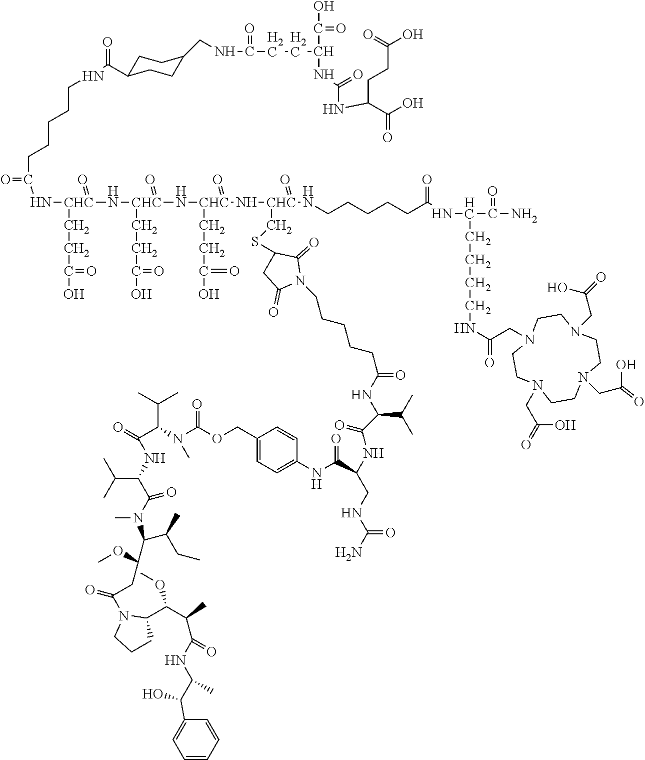

or a pharmaceutically acceptable salt thereof.

The metal ion complexed with the chelating agent can include at least one of a radioactive isotope of Ga, I, In, Y, Lu, Bi, Ac, Re, In, Th, Tc, Tl, Tb, Zr, Cu, Rb, At, Pb, Gd, Sm, or Sr. In other embodiments, the metal ion includes at least one of ¹⁸⁶Re, ¹⁸⁸Re, ^{99m}Tc, ¹⁵³Gd, ¹¹¹In, ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹Tl, ¹⁶¹Tb, ¹⁵³Sm, ⁸⁹Sr, ²¹¹At, ²²⁵Ac, ²²⁷Ac, ¹²³⁻¹²⁵I ¹³¹I, ⁶⁷Cu, ²⁰³Pb, ²¹²Pb, ²¹¹Bi, ²¹³Bi, or ²²⁷⁻²²³Th.

### BRIEF DESCRIPTION OF THE DRAWINGS

The following is a brief description of the drawings which are presented for the purpose of illustrating the invention and not for the purpose of limiting them.

FIGS. 1(A-E) illustrate a schematic and plots showing novel PSMA-targeted drug conjugates. (A) Structure of PSMA-1-VcMMAE-Cy5.5 which has a cleavable linker. (B) Structure of PSMA-1-McMMAE-Cy5.5 which has a noncleavable linker. (C) In vitro competition binding results of PSMA-targeted drug conjugates. Values are mean±SD of triplicates. PSMA-1-VcMMAE-Cy5.5 and PSMA-1-McMMAE-Cy5.5 showed similar binding affinity. (D) In vitro cathepsin cleavage of PSMA-targeted drug conjugates. Both conjugates were stable in PBS. In the presence of cathepsin, PSMA-1-VcMMAE-Cy5.5 decomposed rapidly, while PSMA-1-McMMAE-Cy5.5 remained stable. Values are mean±SD of triplicates. (E) In vitro cytotoxicity of PSMA-targeted drug conjugates to PSMA-positive PC3pip cells (solid lines) and PSMA negative PC3flu cells (dashed lines) after 72-hour incubation. PSMA-1-VcMMAE-Cy5.5 was much more potent than PSMA-1-McMMAE in killing PC3pip cells. Presence of cathepsin B inhibitor E64 reduced the activity of PSMA-1-VcMMAE-Cy5.5, suggesting the conjugate functioned as prodrug. Values are mean±SD of 6 replicates.

FIGS. 2(A-B) illustrate images showing in vitro fluorescence studies of PSMA-targeted drug conjugates. (A) In vitro cellular uptake results of PSMA-targeted drug conjugates. Cells were incubated with 50 nM of drug conjugates for 4 hour. Selective uptake was observed only in PC3pip cells and the conjugates were mainly located in lysosomes. Specificity of drug conjugates for PSMA binding was evaluated by including 1 mM of unlabeled PSMA-1 ligand during incubations. Signal in PC3pip cells was significantly competed by PSMA-1, suggesting the binding is specific. Images are taken at 40×. Representative images are shown from three independent experiments. (B) Immuno-detection of α-tubulin. Cells were treated with 5 nM of drugs for 24 hours then fixed and immunofluorescence stained by Alex-488-labeled α-tubulin antibody. Selective disruption in PC3pip cells was only observed when cells were treated with PSMA-1-VcMMAE-Cy5.5. Images were taken at 40×. Representative images are shown from three independent experiments.

FIGS. 3(A-D) illustrate images and plots showing in vivo biodistribution and antitumor activity studies. (A) In vivo Maestro imaging of a typical mouse bearing heterotopic PC3pip and PC3flu tumors treated with 40 nmol/kg of drug conjugates through i.v. injection. Representative images are shown of n=5. Selective uptake was observed in PC3pip tumors. (B) Quantification of fluorescent signal intensity in PC3pip and PC3flu tumors. Values are mean±SD of 5 animals. (C) Ex vivo imaging of mouse organs at 72-hour post-injection. Fluorescent signal in PC3pip tumor was significantly higher than in other organs. Representative images are shown of n=5. (D) In vivo tumor inhibition of PSMA-targeted drug conjugates using heterotopic PC3pip tumors. Mice received 160 nmol/kg of either PSMA-1-VcMMAE-Cy5.5 or PSMA-1-McMMAE-Cy5.5 every 4 days with a total of 5 doses. Significant tumor regression was only observed in mice treated with PSMA-1-VcMMAE-Cy5.5. Values represent mean±SD of 5 animals. (*, PSMA-1-VcMMAE-Cy5.5 vs PBS, P<0.05; #, PSMA-1-VcMMAE-Cy5.5 vs PSMA-1-McMMAE-Cy5.5, P<0.05).

FIGS. 4(A-B) illustrate plots showing in vitro cytotoxicity studies of PSMA-1-VcMMAE (A) and PSMA-1-VcMMAE-Cy5.5. (B) Cells were incubated with various concentration of drugs for 72 hours, then cell viability was determined by CCK8 assay. Both conjugates showed the ability to selectively kill PC3pip cells. In the presence of 10 M of PSMA-1 ligand, their potency to kill PC3pip cells was reduced to the same as PC3flu cells, indicating the killing is selective for PSMA expression. Presence of cathepsin B inhibitor E64 also reduced the activity of PSMA-1-VcMMAE, suggesting the conjugate functioned as prodrug. Values are mean±SD of 6 replicates.

FIGS. 5(A-C) are a schematic illustration of mechanism of the multifunctional theranostic molecule PSMA-1-MMAE-IR700. After administration, PSMA-1-MMAE-IR700 will selectively bind to PSMA receptors on prostate cancer cells and enter cancer cells through receptor mediated endocytosis. The fluorescence signal emitted by IR700 can be used for diagnosis and image-guided surgery of prostate cancer by detecting residual cancer (A). Internalized PSMA-1-MMAE-IR700 will be delivered to lysosomes, the conjugate will be digested by cathepsins to generate free MMAE and PSMA-1-IR700. Delivered as a prodrug, protease released MMAE will exert its chemotherapeutic effect (B), while PSMA-1-IR700 will generated reactive oxygen species and deploy photodynamic therapy when illuminated by 690 nm light (C).

FIGS. 6(A-D) illustrate characterization of PSMA-1-MMAE-IR700. (A) Structure of PSMA-1-MMAE-IR700. (B) Absorbance spectrum of PSMA-1-MMAE-IR700. (C) In vitro competition binding results of PSMA-1-MMAE-IR700. Values are mean±SD of triplicates. (D) In vitro cathepsin cleavage of PSMA-1-MMAE-IR700. Values are mean±SD of triplicates.

FIG. 7 illustrates in vitro uptake studies of PSMA-1-MMAE-IR700 in PSMA-positive PC3pip and PSMA-negative PC3flu cells. Cells were incubated with 50 nM of PSMA-1-MMAE-IR700 for various times. Blocking experiments were performed by co-incubation of cells with 50 nM of PSMA-1-MMAE-IR700 and 100× of PSMA-1 ligand. Nuclei were stained by DAPI and false colored blue. PSMA-1-MMAE-IR700 signal was false colored red. Images were taken at 40×. Scale bar=50 μm. Representative images are shown from three independent studies.

FIGS. 8(A-B) illustrate in vitro cytotoxicity of PSMA-1-MMAE-IR700. (A) Dark cytotoxicity of PSMA-1-MMAE-IR700. Cells were incubated with drugs for 24 h in the dark, and then cell viability was determined. NA means IC50 value is not available. Values are mean±SD of six replicates. (B) Cytotoxicity of PSMA-1-MMAE-IR700 with 690 nm light treatment. Cells were incubated with 5 nM of drugs for 24 h in the dark. Drugs were then washed off and cell were exposed to 1 J/cm² or 3 J/cm² light. Cell viability was measured 24 h later. Values are mean±SD of six replicates. (*: P<0.0001, PSMA-1-MMAE-IR700 with 1 J/cm² light versus PSMA-1-MMAE-IR700 with no light, or PSMA-1-IR700 with 1 J/cm² light. $: P<0.0001, PSMA-1-MMAE-IR700 with 1 J/cm² light to PC3pip cells versus that treatment to PC3flu cells. #: P<0.0001, PSMA-1-MMAE-IR700 with 3 J/cm² light versus PSMA-1-MMAE-IR700 with no light, or PSMA-1-IR700 with 3 J/cm² light. &: P<0.0001, PSMA-1-MMAE-IR700 with 3 J/cm² light to PC3pip cells versus that treatment to PC3flu cells).

FIGS. 9(A-D) illustrate in vivo fluorescence images of PSMA-1-MMAE-IR700. (A) In vivo Maestro imaging of a typical mouse bearing heterotopic PC3pip and PC3flu tumors treated with 100 nmol/kg of PSMA-1-MMAE-IR700 delivered through i.v. injection. Representative images are shown of n=5. Selective uptake was observed in PC3pip tumors. (B) Quantification of fluorescent signal intensity in PC3pip and PC3flu tumors. Values are mean±SD of 5 animals. (C) Ex vivo imaging of mouse organs at 48 h post injection. Fluorescence from PC3pip tumors was significantly higher than in other organs. Representative images are shown of n=5. (D) Quantification of fluorescent signal intensity in tissues. Values are mean±SD of 5 animals.

FIGS. 10(A-B) illustrate detection of primary orthotopic prostate tumor and lymph node metastases by PSMA-1-MMAE-IR700. Representative images are shown from 3 animals. (A) In vivo and ex vivo fluorescence image of PSMA-1-MMAE-IR700 in mice bearing orthotopic PC3pipGFP tumor. Mice received 100 nmol of PSMA-1-MMAE-IR700 through tail vein injection. Images were taken at 1 h post injection. White arrow indicates lymph node, and red arrow indicates residual primary tumor. (B) Histological analysis of dissected primary tumor and lymph nodes. Presence of tumor cells was confirmed by H&E staining, GFP signal, and IR700 signal from PSMA-1-MMAE-IR700. Nuclear stain, DAPI, is false colored blue. Scale bar=100 μm in the lower panel. Black or white dashed lines indicate the borderline between normal tissue and cancer tissues. “T” is for tumor tissues; “N” is for normal prostate; and “L” is for lymphocytes.

FIGS. 11(A-D) illustrate in vivo antitumor activity of PSMA-1-MMAE-IR700 in mice bearing heterotopic PC3pip tumors. For survival experiments, mice received drugs through tail vein injection. PDT was performed at 1 hour post injection. Treatment was scheduled every 4 days with a total of five doses as indicated by the red arrows. Each group had 5 mice. For tumor growth curves and body weight curves, values are mean±SD of 5 animals. The plots stopped when animals died during the experiments since values are represent as mean±SD of 5 animals. (A) Tumor growth curves of mice. (B) Kaplan-Meier survival curves of treated mice. (*, P<0.05, PSMA-1-MMAE-IR700+PDT versus the other 6 groups. *, P<0.05, PSMA-1-MMAE-IR700 without PDT versus PBS, PSMA-1-IR700 without PDT and PSMA-1-IR700+MMAE without PDT. #, P<0.05, PSMA-1-IR700 with PDT versus PBS, PSMA-1-IR700 without PDT and PSMA-1-IR700+MMAE without PDT; ø, P<0.05, PSMA-1-IR700+MMAE with PDT versus PBS, PSMA-1-IR700 without PDT and PSMA-1-IR700+MMAE without PDT). (C) Body weight changes of mice treated with PSMA-1-VcMMAE. (D) Induction of apoptosis by the treatment. Tumors were dissected at 4 days post treatment and examined by H&E staining and caspase-3 assay. Scale bar=100 μm. Pictures are representative images of five mice.

FIGS. 12(A-E) illustrate PSMA-AuNC-MMAE conjugates characterization. (A) TEM image of PSMA-AuNC-MMAE conjugates. (B) Hydrodynamic size distribution of PSMA-AuNC-MMAE conjugates with average size of 4.4 nm. (C) Excitation and emission spectra of PSMA-AuNC-MMAE conjugates. (D) Fluorescence of PSMA-AuNC-MMAE conjugates at concentration from 0 to 5 mg/mL. (E) Release of MMAE from the PSMA-AuNC-MMAE conjugates upon cathepsin cleavage, with and without protease inhibitor.

FIGS. 13(A-D) illustrate in vitro cell targeting, cytotoxicity and radiosensitization. (A) Selective uptake of PSMA-AuNC-MMAE conjugates and α-tubulin disruption upon intracellular MMAE release (scale bar=30 μm). (B) Cytotoxicity of cells after incubating with PSMA-AuNCs, free MMAE, PSMA-MMAE, PSMA-AuNC-MMAE conjugates, and PSMA-AuNC-MMAE conjugates with protease inhibitor for 24 h (data are presented as mean±SD, n=5; difference is compared with two-tailed t test, **p≤0.01). (C, D) Colony survival curves of PC3pip (C) and PC3flu cells (D) after incubating with PSMA-AuNCs, PSMA-AuNC-MMAE conjugates, PSMA-MMAE, mixture of PSMA-AuNCs and PSMA-AuNC-MMAE with equivalent Au and MMAE (data are presented as mean±SD, n=3; difference is compared with two-tailed t test, **p≤0.01).

FIGS. 14(A-E) illustrate tumor targeting, biodistribution, and clearance of PSMA-AuNCs. (A) Tumor targeting of PSMA-AuNCs for mice bearing PC3pip and PC3flu tumors. (B) Accumulation and kinetics of PSMA-AuNCs in PC3pip and PC3flu tumors after injection. (C) Fluorescence imaging of main organs at 24 h postinjection. (D) Biodistribution of PSMA-AuNCs in main organs at 24 h postinjection. (E) Renal clearance of PSMA-AuNCs before and after injection. Data are presented as mean±SD n=3; two-tailed t-test: *P<0.05.

FIGS. 15(A-D) illustrate tumor growth curve of mice (A) injected with PBS, PSMA-AuNCs, PSMA-MMAE, PSMA-AuNC-MMAE conjugates, and mixture of PSMA-AuNCs and PSMA-MMAE with equivalent Au and MMAE. (B) Mice receiving the same injection and additionally 6 Gy radiation at 4 h postinjection. (C) Tumor weight at the end of observation. Data are presented as mean±SD (n=5), and difference is compared with two-tailed t test, **p≤0.01. (D) Representative tumor H&E and immunochemistry images showing the accumulation of NCs in tumors and the damage induced by MMAE. Top panel: NCs in H&E tumor tissue stained with a silver staining kit; second panel: TUNEL assay of tumor tissues taken from the black box regions demonstrating increased apoptosis; third panel: NCs inherent fluorescence images showing their distribution in tumor tissues; bottom panel: α-tubulin staining showing the disruption of α-tubulin for tumor tissues. Each group of mice were injected with PBS, PSMA-AuNCs, PSMA-AuNC-MMAE conjugates, PSMA-MMAE, and a mixture of PSMA-AuNCs and PSMA-MMAE with equivalent Au and MMAE to that delivered from dosing with PSMA-AuNC-MMAE. Tumors were isolated at 24 h post injection.

FIGS. 16(A-C) illustrate PSMA-AuNC-MMAE enhances sensitivity of the human prostate PC3pip cells to ionizing radiation in vivo. The groups were injected with PBS, PSMA-AuNCs, PSMA-AuNC-MMAE conjugates, PSMA-MMAE, and a mixture of PSMA-AuNCs and PSMA-MMAE with equivalent Au and MMAE to the other agents. Mice received 6 Gy of radiation at 4 h postinjection focused on the tumor region only and tumors were isolated 1 h after the radiation. (A) H&E staining of PC3pip frozen tumor tissue after different treatments. Scale bars=1 mm. (B, C) Immunohistochemistry for the phosphorylated y-H2AX (on serine-139) histones in tumor cell nuclei. Presence of y-H2AXpos nuclei is shown in viable portions of the tumors in (A). Red dotted spots: y-H2AXpos staining. Blue nuclei: DAPI. Arrows: y-H2AXpos nuclei. Inset images are single cells at high magnification. Scale bars=25 μm. (Necrotic tumor areas showed disorganized y-H2AXpos staining for DNA breaks; data not shown.)

FIGS. 17(A-C) illustrate (A) Molecular Structure of CY-PSMA-1-MMAE Ligand, (B) PSMA-AuNC-MMAE Conjugates Formation Using the CY-PSMA-1-MMAE Ligands, and (C) Uptake Sequence of PSMA-AuNC-MMAE Conjugates, Intracellular MMAE Release from Lysosomes, Radiosensitization by Gold (1) and Chemotherapy by Free MMAE via a Microtubule Disruption Mechanism (2)a aMMAE arrests DNA in a form more susceptible to destruction by RT.

FIG. 18 illustrates synthesis of CY-PSMA-MMAE-SH conjugation. (1) Peptide CY-PSMA-1 was synthesized by Fmoc chemistry and conjugated with MMAE via a Vc linker; (2) —NH₂ group in the Lys residue was reacted with N-succinimidyl S-acetylthioacetate (SATA) to introduce active —SH group in the conjugate; (3) SATA was deprotected to produce free —SH group.

FIG. 19 illustrates synthesis of C(Trt)Y-PSMA-SH. Peptide C(Trt)Y-PSMA-1 was synthesized by Fmoc chemistry and (1) the —NH₂ group in the Lys residue was reacted with N-succinimidyl S-acetylthioacetate (SATA) to introduce active —SH group in the conjugate; (2) SATA was deprotected to produce free —SH group. To synthesize the conjugates with similar reactive —SH group from the Lys residue, the original Cys residue was protected with Trt.

FIGS. 20(A-C) illustrate (A) TEM image of PSMA-AuNCs-MMAE at low magnification (inset, thon ring for the NCs; scale bar=20 nm); (B) Emission spectra of the reaction mixture indicating the formation of PSMA-AuNC-MMAE conjugates over time at room temperature; (C) PSMA-AuNC-MMAE conjugates synthesized at room temperature and at 37° C.

FIG. 21 illustrates α-tubulin staining for the blank PC3pip and PC3flu cells, showing the perfect green fibrous α-tubulin network and spindle structure.

FIG. 22 illustrates in vitro uptake and α-tubulin staining for PC3pip at different time point with E64 inhibitor added and incubating with PSMA-AuNC-MMAE conjugates, showing the E64 inhibitor could inhibit the cleavage of MMAE from the PSMA-AuNC-MMAE conjugates and reduce the toxicity to cells. At 1 h and 6 h after coincubation with AuNCs-MMAE, α-tubulin network for PC3pip cells was as normal as the blank control. However, at 24 h, their structure was still disrupted but compared to cells with no addition of E64 inhibitor, cell morphology was slightly better.

FIGS. 23(A-B) illustrate cytotoxicity of cells after incubating with PSMA-AuNCs, free MMAE, PSMA-MMAE, PSMA-AuNC-MMAE conjugates, and PSMA-AuNC-MMAE conjugates with inhibitor for (a) 1 h and (b) 4 h. Data were presented as mean±SD (n=5).

FIG. 24 illustrates biodistribution of PSMA-AuNCs in main organs at 24 h post-injection (ICP). Data are presented as mean±SD. n=3; two-tailed t-test: *P<0.05.

FIGS. 25(A-B) illustrate body weight of mice (A) injected with PBS, PSMA-AuNCs, PSMA-MMAE, PSMA-AuNC-MMAE conjugates, and mixture of PSMA-AuNCs &PSMA-MMAE with equivalent Au and MMAE; (B) mice receiving the same injection and additionally 6 Gy radiation at 4 h post-injection. Data were presented as mean±SD (n=5).

FIG. 26 illustrates a graph showing statistical analysis of the levels of γ-H2AX^{pos} nuclei.

FIG. 27 illustrates histology analysis of main organs from mice injected with PBS (blank control) and PSMA-targeted AuNCs-MMAE at the end of treatment (day 30).

FIG. 28 illustrates a synthesis scheme for PSMA-targeted MR contrast agents.

FIGS. 29(A-C) illustrate Maestro Image data of mice receiving 1 nmol of PSMA-1-Cys(Cy5.5)-C6-Lys(DOTA-Gd)2. (A) In vivo images at different time points. (B) Ex vivo imaging of organs. (C) Quantification of fluorescent signal in CP3pip and PC3flu tumors. Values represent mean±SD of 3 animals.

FIGS. 30(A-C) illustrate Maestro Image data of mice receiving 1 nmol of PSMA-1-Cys(Cy5.5)-C6-Lys(DOTA-Gd)4. (A) In vivo images at different time points. (B) Ex vivo imaging of organs. (C) Quantification of fluorescent signal in CP3pip and PC3flu tumors. Values represent mean±SD of 3 animals.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the application.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,” as used herein are non-limiting and are for illustrative purposes only. “Including” and “including but not limited to” are used interchangeably.

The term “or” as used herein should be understood to mean “and/or” unless the context clearly indicates otherwise.

The term “agent” is used herein to denote a chemical compound, a mixture of chemical compounds, a biological macromolecule, or an extract made from biological materials.

The term “sample” can refer to a specimen or culture obtained from any source, as well as clinical, research, biological and environmental samples. Biological samples may be obtained from animals (including humans) and encompass cells, fluids, solids, tissues, and organs, and whole organisms.

The terms “patient”, “subject”, “mammalian host,” and the like are used interchangeably herein, and refer to humans and non-human animals (e.g., rodents, arthropods, insects, fish (e.g., zebrafish)), non-human primates, ovines, bovines, ruminants, lagomorphs, porcines, caprines, equines, or canines felines, aves, etc.).

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys, and liver.

Terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “polypeptide” refers to a polymer composed of amino acid residues related naturally occurring structural variants, and synthetic non-naturally occurring analogs thereof linked via peptide bonds or modified peptide bonds (i.e., peptide isosteres), related naturally occurring structural variants, and synthetic non-naturally occurring analogs thereof, glycosylated polypeptides, and all “mimetic” and “peptidomimetic” polypeptide forms. Synthetic polypeptides can be synthesized, for example, using an automated polypeptide synthesizer. The term can refer to an oligopeptide, peptide, polypeptide, or protein sequence, or to a fragment, portion, or subunit of any of these. The term “protein” typically refers to large polypeptides. The term “peptide” typically refers to short polypeptides.

A “portion” of a polypeptide or protein means at least about three sequential amino acid residues of the polypeptide. It is understood that a portion of a polypeptide may include every amino acid residue of the polypeptide.

“Mutants,” “derivatives,” and “variants” of a polypeptide (or of the DNA encoding the same) are polypeptides which may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

“Recombinant,” as used herein, means that a protein is derived from a prokaryotic or eukaryotic expression system.

The term “derivative” refers to an amino acid residue chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-benzylhistidine. Also included as derivatives are those amino acid residues, which contain one or more naturally occurring amino acid derivatives of the twenty standard amino acids, such as non-standard amino acids.

“PSMA” refers to Prostate Specific Membrane Antigen, a potential carcinoma marker that has been hypothesized to serve as a target for imaging and cytotoxic treatment modalities for cancer.

The term “small molecule” can refer to lipids, carbohydrates, polynucleotides, polypeptides, or any other organic or inorganic molecules.

The term “imaging probe” can refer to a biological or chemical moiety that may be used to detect, image, and/or monitor the presence and/or progression of a cell cycle, cell function/physiology, condition, pathological disorder and/or disease.

The terms “treating” or “treatment” of a disease can refer to executing a treatment protocol to eradicate at least one diseased cell. Thus, “treating” or “treatment” does not require complete eradication of diseased cells.

The term “nanoparticle” refers to any particle having a diameter of less than 1000 nanometers (nm). In some embodiments, nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments. In general, the nanoparticles should have dimensions small enough to allow their uptake by eukaryotic cells. Typically, the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., about 1 nm to about 30 nm or about 1 nm to about 5 nm, are used in some embodiments.

The term “radiosensitizer” refers to compounds or agents that increase the cytotoxicity of ionizing radiation. For example, heavy-metal nanomaterials with high atomic number (Z) values, such as gold nanomaterials.

An “effective amount” can refer to that amount of a therapeutic agent that results in amelioration of symptoms or a prolongation of survival in the subject and relieves, to some extent, one or more symptoms of the disease or returns to normal (either partially or completely) one or more physiological or biochemical parameters associated with or causative of the disease.

Therapeutic agents can include any agent (e.g., molecule, drug, pharmaceutical composition, etc.) capable of preventing, inhibiting, or arresting the symptoms and/or progression of a disease.

Embodiments described herein relate to PSMA targeted conjugate compounds, pharmaceutical compositions comprising these compounds, methods for treating and detecting cancers (e.g., prostate cancer) in a subject using these PSMA targeted conjugate compounds, and methods for identifying cancer cells (e.g., prostate cancer cells) in a sample using these compounds. It has been shown that PSMA targeted compounds conjugated to anti-cancer agents can increase uptake of the conjugate compound in PSMA expressing cells while also improving cell killing compared to agents administered alone. In addition, PSMA targeted conjugate compounds described herein can decrease non-PSMA target toxicity of the therapeutic agent, or a theranostic agent administered (e.g., systemically) to a subject. Moreover, surprisingly it was found that the PSMA targeted conjugate compound upon systemic administration to a subject show minimal accumulation and/or uptake in non-PSMA targets in non-cancer tissue, such as salivary glands, lacrimal glands, and kidney of the subject.

Pathological studies indicate that PSMA is expressed by virtually all prostate cancers, and its expression is further increased in poorly differentiated, metastatic, and hormone-refractory carcinomas. Higher PSMA expression is also found in cancer cells from castration-resistant prostate cancer patients. Increased PSMA expression is reported to correlate with the risk of early prostate cancer recurrence after radical prostatectomy. In addition to being overexpressed in prostate cancer (PCa), PSMA is also expressed in the neovasculature of neoplasms including but not limited to conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma.

In some embodiments, the PSMA targeted conjugate compounds described herein, can selectively recognize PSMA-expressing tumors, cancer cells, and/or cancer neovasculature in vivo and be used to deliver a therapeutic agent, detectable moiety, and/or theranostic agent to the PSMA-expressing tumors, cancer cells, and/or cancer neovasculature to treat and/or detect the PSMA-expressing tumors, cancer cells, and/or cancer neovasculature in a subject.

In some embodiments, the PSMA expressing cancer that is treated and/or detected is prostate cancer. In other embodiments, the cancer that is treated and/or detected can include malignant neoplasms, such a conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma.

In some embodiments, the compound can include the general formula (I):

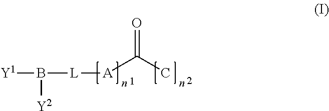

wherein:
n¹ and n² are each independently 1, 2, 3, or 4;
A, B, and C each independently include at least one of a negatively charged amino acid, an acidic amino acid, a non-standard negatively charged or acidic amino acid thereof, a derivative thereof, or an analogue thereof;
L is an optionally substituted aliphatic or heteroaliphatic linking group; and
Y¹ and Y² are each independently a H of B or can include at least one detectable moiety, therapeutic agent, or a theranostic agent that is directly or indirectly linked to B, wherein at least one of Y¹ or Y² is not H.

In some embodiments, Y¹ and Y² are not H.

In some embodiments, the compound of formula (I) does not bind to aquaporin (e.g., AQP3), such as aquaporin expressed in the salivary glands, upon administration of the compound to a subject with cancer.

In other embodiments, the compound has a selectivity for PSMA expressing cancer tissue versus non-PSMA expressing non-cancer tissue ≥5 times, ≥10 times, ≥20 times, ≥30 times, ≥40 times, ≥50 times or more times.

In other embodiments, the compound upon administration to a subject in need thereof shows minimal accumulation or uptake in salivary glands of the subject.

In other embodiments, Y¹ and Y² can include at least one radiolabel or radioisotope, radiosensitizer, chelating agent, photosensitizer, fluorescent labeling agent, magnetic resonance imaging (MRI) agent, or anti-cancer agent.

In some embodiments, the anticancer agent is linked to B via a protease cleavable or acid-labile linker. The linker can include a maleimido-caproyl linker-valine-citrulline cleavable peptide-p-aminobenzyl carbamate spacer (MC-VC-PABC) protease cleavable linker.

In some embodiments, at least one of Y¹ or Y² includes a chelating agent optionally complexed with a metal or metal ion. In some embodiments, the chelating agent includes at least one of diethylenetriaminepentaacetate (DTPA), 1,4,7,10-tetraazadodecanetetraacetate (DOTA), 1,4,7,10-tetraazadodecane-1,4,7-triacetate (DO3A), ethylenediaminetetraacetate (EDTA), 1,4,7,10-tetraazacyclotridecanetetraacetic acid (TRITA), 1,4,8,11-tetraazacyclotetradecane-1,4,8,11-tetraacetic acid (TETA), 1,4,7,10-tetraazadodecanetetramethylacetate (DOTMA), 1,4,7,10-tetraazadodecane-1,4,7-trimethylacetate (DO3MA), N,N′,N″,N′″-tetraphosphonatomethyl-1,4,7,10-tetraazacyclododecane (DOTP), 1,4,7,10-tetraazacyclododecane-1,4,7,10-tetrakis(methylene methylphosphonic acid) (DOTMP), 1,4,7,10-tetraazacyclododecane-1,4,7,10-tetrakis(methylene phenylphosphonic acid) (DOTPP), N,N′-ethylenedi-L-cysteine, 1,4,7-triazacyclononane-1,4,7-triacetic acid (NOTA), 1,4,7-triazacyclononane (TACN), N,N′-Bis(2-hydroxy-5-(ethylene-beta-carboxy)benzyl)ethylenediamine N,N′-diacetic acid (HBED-CC), S-2-(4-isothiocyanatobenzyl)-1,4,7,10-tetraazacylododecane tetracetic acid (p-SCN-Bn-DOTA), MeO-DOTA-NCS, [(R)-2-Amino-3-(4-isothiocyanatophenyl)propyl]-trans-(S,S)-cyclohexane-1,2-diamine-pentaacetic acid (CHX-A″-DTPA-NCS), 2-[4-nitrobenzyl]-1,4,7,10,13-pentaazacyclopentadecane-N,N′,N″,N′″,N″″-pentaacetic acid (PEPA), 1,4,7,10,13,16-hexaazacyclooctadecane-N,N′,N″,N′″,N″″-hexaacetic acid (HEHA), desferrioxamine B (DFO), or derivatives thereof.

In some embodiments, the metal ion includes at least one of a radioactive isotope of Ga, I, In, Y, Lu, Bi, Ac, Re, In, Th, Tc, Tl, Tb, Zr, Cu, Rb, At, Pb, Gd, Sm, or Sr. In other embodiments, the metal ion includes at least one of ¹⁸⁶Re, ¹⁸⁸Re, ^{99m}Tc ¹⁵³Gd, ¹¹¹In ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹P, ¹⁶¹T, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th.

In other embodiments, L can be an optionally substituted aliphatic or heteroaliphatic group that includes at least one ring selected from the group consisting of an optionally substituted 4 to 7 membered nonaromatic heterocyclic ring and an optionally substituted C4-C7 cycloalkyl ring.

An aliphatic group is a straight chained, branched or cyclic non-aromatic hydrocarbon, which is completely saturated or which contains one or more units of unsaturation. An alkyl group is a saturated aliphatic group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. C1-C4 straight chained or branched alkyl or alkoxy groups or a C3-C8 cyclic alkyl or alkoxy group (preferably C1-C4 straight chained or branched alkyl or alkoxy group) are also referred to as a “lower alkyl” or “lower alkoxy” groups; such groups substituted with —F, —Cl, —Br, or —I are “lower haloalkyl” or “lower haloalkoxy” groups; a “lower hydroxyalkyl” is a lower alkyl substituted with —OH; and the like.

Optional substituents for a substitutable atom in alkyl, cycloalkyl, aliphatic, cycloaliphatic, heterocyclic, benzylic, aryl, or heteroaryl groups described herein are those substituents that do not substantially interfere with the activity of the disclosed compounds. A “substitutable atom” is an atom that has one or more valences or charges available to form one or more corresponding covalent or ionic bonds with a substituent. For example, a carbon atom with one valence available (e.g., —C(—H)═) can form a single bond to an alkyl group (e.g., —C(-alkyl)=), a carbon atom with two valences available (e.g., —C(H₂)—) can form one or two single bonds to one or two substituents (e.g., —C(alkyl)(Br))—, —C(alkyl)(H)—) or a double bond to one substituent (e.g., —C═O)—), and the like. Substitutions contemplated herein include only those substitutions that form stable compounds.

For example, suitable optional substituents for substitutable carbon atoms include —F, —Cl, —Br, —I, —CN, —NO₂, —OR^{a}, —C(O)R^{a}, —OC(O)R^{a}, —C(O)OR^{a}, —SR^{a}, —C(S)R^{a}, —OC(S)Ra, —C(S)OR^{a}, —C(O)SR^{a}, —C(S)SR^{a}, —S(O)R^{a}, —SO₂R^{a}, —SO₃R^{a}, POR^{a}R^{b}, PO₂R^{a}R^{b}, —PO₃R^{a}R^{b}, —PO₄R^{a}R^{b}, —P(S)R^{a}R^{b}, —P(S)OR^{a}R^{b}, —P(S)O₂R^{a}R^{b}, —P(S)O₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}) —SO₂N(R^{a}R^{b}), —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C═CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl, wherein R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group. Also contemplated are isomers of these groups.

Suitable substituents for nitrogen atoms having two covalent bonds to other atoms include, for example, optionally substituted alkyl, optionally substituted cycloalkyl, optionally substituted aliphatic, optionally substituted cycloaliphatic, optionally substituted heterocyclic, optionally substituted benzyl, optionally substituted aryl, optionally substituted heteroaryl, —CN, —NO₂, —OR^{a}, —C(O)R^{a}, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), and the like.

Suitable substituents for nitrogen atoms having three covalent bonds to other atoms include —OH, alkyl, and alkoxy (preferably C1-C4 alkyl and alkoxy). Substituted ring nitrogen atoms that have three covalent bonds to other ring atoms are positively charged, which is balanced by counteranions such as chloride, bromide, fluoride, iodide, formate, acetate and the like. Examples of other suitable counter anions are provided in the section below directed to suitable pharmacologically acceptable salts.

In some embodiments B includes at least one, two, three, four, five, or more negatively charged or acidic amino acids.

In some embodiments, the negatively charged or acidic amino acids can be selected from aspartic acid or aspartate or glutamic acid or glutamate.

In other embodiments, B further includes at least one of lysine, tyrosine, or cysteine.

In other embodiments, the compound can include the general formula (II):

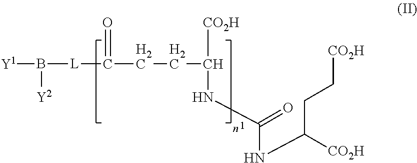

wherein:
n¹ is 1, 2, 3, or 4;
B includes a negatively charged amino acid, an acidic amino acid, a non-standard negatively charged or acidic amino acid thereof, a derivative thereof, or an analogue thereof;
L is an optionally substituted aliphatic or heteroaliphatic linking group; and
Y¹ and Y² are each independently a H of B or can include at least one detectable moiety, therapeutic agent, or a theranostic agent that is directly or indirectly linked to B, wherein at least one of Y¹ or Y² is not H.

In some embodiments, Y¹ and Y² are not H.

In some embodiments, Y¹ and Y² each independently include at least one radiolabel or radioisotope, radiosensitizer, chelating agent, photosensitizer, fluorescent labeling agent, magnetic resonance imaging (MRI) agent, or anti-cancer agent.

In some embodiments, B has the following formula:

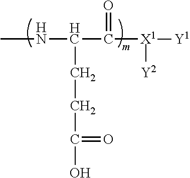

wherein m is 1, 2, 3, or 4, X¹ includes at least one amino acid, and at least one of Y¹ or Y² includes at least one detectable moiety, therapeutic agent, or theranostic agent that is directly or indirectly linked to X¹.

In some embodiments, X¹ further includes at least one of lysine, tyrosine, or cysteine.

In some embodiments, two of Y¹ and Y² include at least one radiolabel or radioisotope, radiosensitizer, chelating agent, photosensitizer, fluorescent labeling agent, magnetic resonance imaging (MRI) agent, or anti-cancer agent

In other embodiments, B has the following formula:

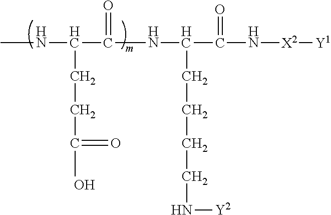

wherein m is 1, 2, 3, or 4;
X² is H or includes at least one amino acid;
Y¹ includes at least one detectable moiety; therapeutic agent, or theranostic agent that is directly or indirectly linked to X² or is absent if X² is H; and
Y² includes at least one detectable moiety, therapeutic agent, or theranostic agent.

In other embodiments, one of Y¹ or Y² includes at least one radiolabel or radioisotope, radiosensitizer, chelating agent, photosensitizer, fluorescent labeling agent, magnetic resonance imaging (MRI) agent, or anti-cancer agent.

In some embodiments, the compound can include the general formula:

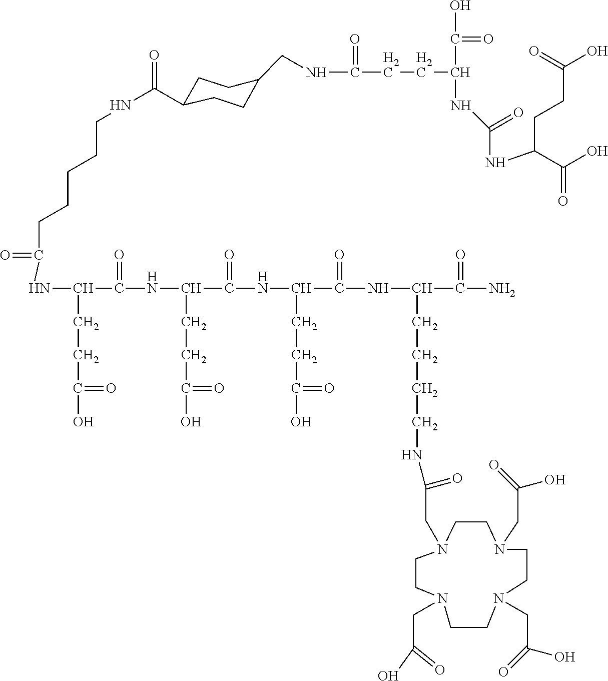

complexed with at least one metal contrast agent or metal ion. In some embodiments, the metal ion can include at least one of ¹⁸⁶Re, ¹⁸⁸Re, ^{99m}Tc, ¹⁵³Gd, ¹¹¹In, ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹T, ¹⁶¹Tb, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th or a pharmaceutically acceptable salt thereof.

In some embodiments, the compound includes the following formula:

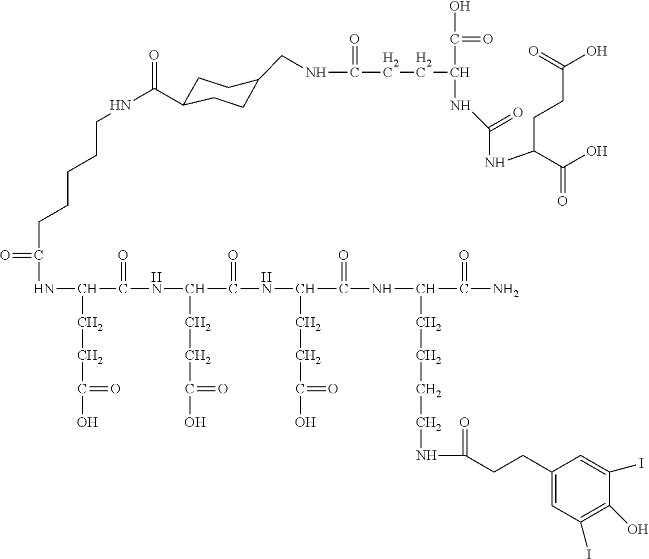 [SMILES: NC(=O)C(CCCCNC(=O)CCc1cc(I)c(O)c(I)c1)NC(=O)C(CCC(=O)O)NC(=O)C(CCC(=O)O)NC(=O)C(CCC(=O)O)NC(=O)CCCCCNC(=O)C1CCC(CNC(=O)CCC(NC(=O)NC(CCC(=O)O)C(=O)O)C(=O)O)CC1]

or a pharmaceutically acceptable salt thereof, wherein the I is ¹²³⁻¹²⁵I or ¹³¹I.

In some embodiments, B has the following formula:

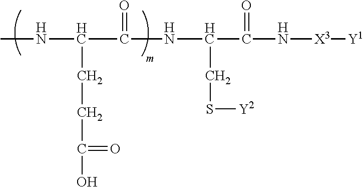

wherein m is 1, 2, 3, or 4;
X³ is H or includes at least one of an amino acid or linker;
Y¹ includes at least one of H, detectable moiety, therapeutic agent, or theranostic agent that is directly or indirectly linked to X³, or is absent if X³ is a H; and
Y² includes at least one detectable moiety, therapeutic agent, or theranostic agent.

In other embodiments, Y¹ and Y² include at least one radiolabel or radioisotope, radiosensitizer, chelating agent, photosensitizer, fluorescent labeling agent, magnetic resonance imaging (MRI) agent, or anti-cancer agent.

In some embodiments, the radiosensitizer is a Au ion that is conjugated to a free thiol.

In some embodiments, the compound includes the following formula:

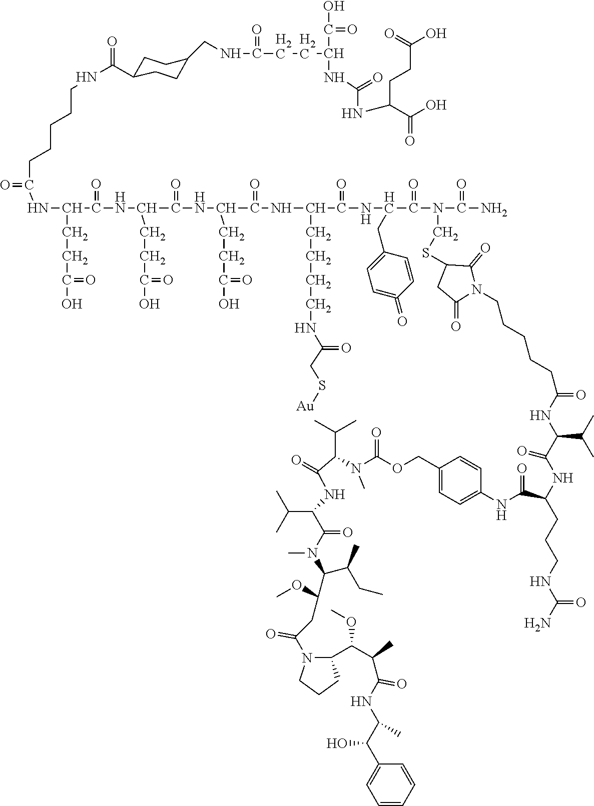

In some embodiments, a plurality of such compounds can aggregate form a PSMA targeted gold nanocluster (AuNC) and MMAE conjugates (PSMA-AuNC-MMAE), for radiotherapy and chemotherapy. In an exemplary embodiment, PSMA-AuNC-MMAE synthesis can occur by CY-PSMA-1-MMAE ligand reduction and stabilization where the cysteine residue captures Au³⁺ ions in solution with the thiol group and a pH increase from the addition of NaOH in the solution turns the phenolic group of the tyrosine residue into a phenoxide ion, which can reduce Au³⁺ ions. The resultant Au atoms can then aggregate to form gold nanoclusters where the gold nanoclusters are stabilized via thiol groups of the CY-PSMA-1-MMAE cysteine residues.

The PSMA-AuNC-MMAE can exhibit discrete optical and electronic states characterized by, for example, strong quantum effects and fluorescence, which allows the gold nanoclusters to behave as molecular, rather than metallic substances. This molecular optical and electronic behavior sharply distinguishes the PSMA-AuNC-MMAE from gold nanoparticles whose optical characteristics can be driven by plasmon resonance.

In some embodiments, an PSMA-AuNC-MMAE formed from a plurality of compounds that include formula (I) as described herein can each include about 10 to about 150 gold atoms. In some embodiments, the gold nanoclusters include about 10 to about 40 gold atoms or about 10 to about 12 gold atoms. In particular embodiments, a gold nanocluster can include 15, 18, 22, 25, 39 or 144 gold atoms. In an exemplary embodiment, a PSMA-AuNC-MMAE formed from a plurality of compounds as described herein can include 25 gold atoms.

In some embodiments, PSMA-AuNC-MMAE for use in a composition described herein can have an average diameter less than about 10 nm. In order to reduce nanoparticle-induced toxicity compared to larger particles, PSMA-AuNC-MMAE formed as described herein can be small enough (e.g., less than about 5.5 nm) to be excreted from urine. In some embodiments, PSMA-AuNC-MMAE for use in a composition describe herein can have an average diameter less than about 5 nm. In particular embodiments, a PSMA-AuNC-MMAE can have an average diameter of about 1.5 nm. An exemplary PSMA-AuNC-MMAE for use in a composition describe herein can have an average diameter of 1.5±0.5 nm and a hydrodynamic diameter of 3.0 nm.

In other embodiments, the compound can include the general formula:

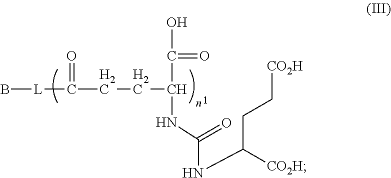

wherein n¹ is independently 1, 2, 3, or 4;
L is an optionally substituted aliphatic or heteroaliphatic linking group;
wherein B has the following formula:

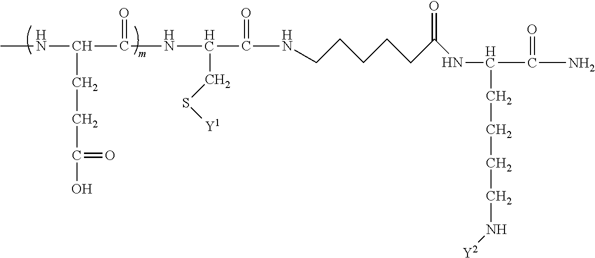

wherein m is 1, 2, 3, or 4; and
Y¹ is a first imaging agent, therapeutic agent, or theranostic agent that is directly or indirectly linked to the thiol group and Y² is a second imaging agent, therapeutic agent, or theranostic agent that is directly or indirectly linked to the amine group.

In some embodiments, each of Y¹ or Y² independently includes at least one radiolabel or radioisotope, radiosensitizer, chelating agent, photosensitizer, fluorescent labeling agent, magnetic resonance imaging (MRI) agent, or anti-cancer agent.

In some embodiments, L can include at least one ring selected from the group consisting of an optionally substituted 4 to 7 membered nonaromatic heterocyclic ring and an optionally substituted C4-C7 cycloalkyl ring.

In some embodiments, at least one of Y¹ or Y² includes a chelating agent optionally complexed with a metal contrast agent or metal ion.

In some embodiments, the chelating agent includes at least one of diethylenetriaminepentaacetate (DTPA), 1,4,7,10-tetraazadodecanetetraacetate (DOTA), 1,4,7,10-tetraazadodecane-1,4,7-triacetate (DO3A), ethylenediaminetetraacetate (EDTA), 1,4,7,10-tetraazacyclotridecanetetraacetic acid (TRITA), 1,4,8,11-tetraazacyclotetradecane-1,4,8,11-tetraacetic acid (TETA), 1,4,7,10-tetraazadodecanetetramethylacetate (DOTMA), 1,4,7,10-tetraazadodecane-1,4,7-trimethylacetate (DO3MA), N,N′,N″,N′″-tetraphosphonatomethyl-1,4,7,10-tetraazacyclododecane (DOTP), 1,4,7,10-tetraazacyclododecane-1,4,7,10-tetrakis(methylene methylphosphonic acid) (DOTMP), 1,4,7,10-tetraazacyclododecane-1,4,7,10-tetrakis(methylene phenylphosphonic acid) (DOTPP), N,N′-ethylenedi-L-cysteine, 1,4,7-triazacyclononane-1,4,7-triacetic acid (NOTA), 1,4,7-triazacyclononane (TACN), N,N′-Bis(2-hydroxy-5-(ethylene-beta-carboxy)benzyl)ethylenediamine N,N′-diacetic acid (HBED-CC), S-2-(4-isothiocyanatobenzyl)-1,4,7,10-tetraazacylododecane tetracetic acid (p-SCN-Bn-DOTA), MeO-DOTA-NCS, [(R)-2-Amino-3-(4-isothiocyanatophenyl)propyl]-trans-(S,S)-cyclohexane-1,2-diamine-pentaacetic acid (CHX-A″-DTPA-NCS), 2-[4-nitrobenzyl]-1,4,7,10,13-pentaazacyclopentadecane-N,N′,N″,N′″,N″″-pentaacetic acid (PEPA), 1,4,7,10,13,16-hexaazacyclooctadecane-N,N′,N″,N′″,N″″-hexaacetic acid (HEHA), desferrioxamine B (DFO), or derivatives thereof.

In some embodiments, the metal ion includes at least one of a radioactive isotope of Ga, I, In, Y, Lu, Bi, Ac, Re, In, Th, Tc, Tl, Tb, Zr, Cu, R^{b}, At, Pb, Gd, Sm, or Sr. In other embodiments, the metal ion includes at least one of ¹⁸⁶Re, ¹⁸⁸Re, ^{99m}Tc, ¹⁵³Gd, ¹¹¹In, ⁶⁷Ga, ⁶⁸Ga, ²⁰¹Tl, ⁸²Rb, ¹¹¹Cu, ⁸⁹Zr, ⁹⁰Y, ¹⁷⁷Lu, T(tritium), ¹⁴⁹T, ¹⁶¹Tb, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th.

In some embodiments, the anticancer agent is linked to B via a protease cleavable or acid-labile linker. The linker can include a maleimido-caproyl linker-valine-citrulline cleavable peptide-p-aminobenzyl carbamate spacer (MC-VC-PABC) protease cleavable linker.

In some embodiments, the compound has the general formula:

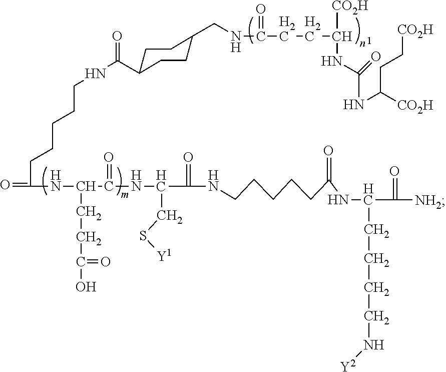

wherein m and n¹ are each independently 1, 2, 3, or 4; and wherein Y¹ includes a radiolabel or radioisotope, radiosensitizer, chelating agent, photosensitizer, fluorescent labeling agent, magnetic resonance imaging (MRI) agent, or anti-cancer agent that is directly or indirectly linked to the thiol group and Y² includes a radiolabel or radioisotope, radiosensitizer, chelating agent, photosensitizer, fluorescent labeling agent, magnetic resonance imaging (MRI) agent, or anti-cancer agent that is directly or indirectly linked to the amine group.

In some embodiments, a therapeutic, imaging and/or a theranostic agent can be coupled directly or indirectly to the PSMA targeted compound via a linker. The linker can include a heterobifunctional crosslinker capable of conjugating to the cysteine residue of the PSMA targeted compound via a maleimide group that is sulfhydryl (thiol; —SH) reactive.

In some embodiments, the heterobifunctional crosslinker can include a protease cleavable or acid-labile linker. In particular embodiments, the detectable moiety, the therapeutic agent, or the theranostic agent can be coupled directly or indirectly to the PSMA targeted compound via an acid-labile linker, such as a 4-(4-maleimidomethyl)cyclohexane-1-carboxyl hydrazide (MMCCH) linker. The MMCCH linker includes a hydrazine bond that can be cleaved to release the detectable moiety, therapeutic agent, or the theranostic agent coupled to the PSMA targeted compound. In alternative embodiments, the detectable moiety, the therapeutic agent, or the theranostic agent can be coupled directly or indirectly to the PSMA targeted portion of the compound (PSMA-1(Cys)) via a non-cleavable linker, such as Succinimidyl 4-(N-maleimidomethyl)cyclohexane-1-carboxylate (SMCC).

In other embodiments, the detectable moiety, therapeutic agent, or the theranostic agent can be coupled directly or indirectly to the PSMA targeted portion of the compound (PSMA-1(Cys)) via a protease cleavable linker. The protease cleavable linker can include a MC-Val-Cit-PABC protease cleavable linker where the Valine-Citrulline-PABC portion is protease cleaveable.

In some embodiments, the PSMA targeted conjugate compounds described herein are prepared by coupling a “vc-MMAE” linker-drug combination construct to the thiol group of the cysteine residue of the PSMA targeted compound. The vc-MMAE construct is also referred to as the linker-drug mc-vc-PABC-MMAE. The construct utilizes a maleimidocaproyl (mc) spacer, a protease-sensitive dipeptide, valine-citrulline (vc), a self-immolative spacer, para-amino benzyloxycarbonyl (PABC), and the antimitotic agent, monomethyl auristatin E (MMAE). The me spacer provides enough room for the vc group to be recognized by the lysosomal cysteine protease cathepsin B in a PSMA expressing cell, which cleaves the citrulline-PABC amide bond. The resultant PABC-substituted MMAE is not a stable intermediate and spontaneously undergoes a 1,6-elimination with a loss of p-iminoquinone methide and carbon dioxide (self-immolation) leaving MMAE as the product to exert anti-mitotic effects.

In some embodiments, the PSMA targeted conjugate compound can include a detectable moiety directly or indirectly coupled to B. Examples of detectable moieties include, but are not limited to: various ligands, imaging agents, contrast agents, radionuclides, fluorescent dyes, near-infrared dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, chelating groups, and biotin, dioxigenin or other haptens and proteins for which antisera or monoclonal antibodies are available.

In one example, the detectable moiety can include a radiolabel, that is directly or indirectly linked (e.g., attached or complexed) with B using general organic chemistry techniques. The detectable peptide can also include radiolabels, such as ⁶⁸Ga, ¹²³, ¹³¹I, ¹²⁵, ¹⁸F, ¹¹C, ⁷⁵Br, ⁷⁶Br, ¹²⁴, ¹³N, ⁶⁴Cu, ³²P, ³S, for PET by techniques well known in the art and are described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, NY 1986) the contents of which are hereby incorporated by reference. The detectable moiety can also include ¹²³I for SPECT. The ¹²³I can be coupled to B can by any of several techniques known to the art. See, e.g., Kulkarni, Int. J. Rad. Appl. & Inst. (Part B) 18: 647 (1991), the contents of which are hereby incorporated by reference. In addition, detectable moiety can include any radioactive iodine isotope, such as, but not limited to ¹³¹I, ¹²⁵, or ¹²³I. The radioactive iodine isotopes can be coupled to B by iodination of a diazotized amino derivative directly via a diazonium iodide, see Greenbaum, F. Am. J. Pharm. 108: 17 (1936), or by conversion of the unstable diazotized amine to the stable triazene, or by conversion of a non-radioactive halogenated precursor to a stable tri-alkyl tin derivative which then can be converted to the iodo compound by several methods well known to the art.

The detectable moiety can further include known metal radiolabels, such as Technetium-99m (^{99m}Tc), ¹⁵³Gd, ¹¹¹In, ⁶⁷Ga, ²⁰¹Tl, ⁸²Rb, ⁶⁴Cu, ⁹⁰Y, ¹⁷⁷Lu¹⁸⁸Rh, T(tritium), ¹⁵³Sm, ⁸⁹Sr, and ²¹¹At, ²²⁷Ac. The metal radiolabeled compounds can then be used to detect cancers, such as prostate cancer in the subject. Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2):135-40, (1998); and Hom et al., “Technetium-99m-labeled receptor-specific small-molecule radiopharmaceuticals: recent developments and encouraging results” Nuclear Medicine & Biology 24(6):485-98, (1997).

In still other embodiments, a radiolabel or radioisotope can be converted from an imaging agent to a radiotherapy. The radiolabel of the PSMA targeting conjugate compound can be injected into the subject's body and targeted to the cancer in the subject by the PSMA targeting conjugate compound. Alpha or gamma irradiation treatment can be used to switch the radiolabel from an imaging isotope of lower energy to a therapeutic agent of higher energy. Switching the isotope converts the agent from an imaging agent to an alpha or gamma emitting therapeutic agent.

In some embodiments, the detectable moiety can include a chelating agent (with or without a chelated metal group). Exemplary chelating agents can include those disclosed in U.S. Pat. No. 7,351,401, which is herein incorporated by reference in its entirety. In some embodiments, the chelating agent includes at least one of diethylenetriaminepentaacetate (DTPA), 1,4,7,10-tetraazadodecanetetraacetate (DOTA), 1,4,7,10-tetraazadodecane-1,4,7-triacetate (DO3A), ethylenediaminetetraacetate (EDTA), 1,4,7,10-tetraazacyclotridecanetetraacetic acid (TRITA), 1,4,8,11-tetraazacyclotetradecane-1,4,8,11-tetraacetic acid (TETA), 1,4,7,10-tetraazadodecanetetramethylacetate (DOTMA), 1,4,7,10-tetraazadodecane-1,4,7-trimethylacetate (DO3MA), N,N′,N″,N′″-tetraphosphonatomethyl-1,4,7,10-tetraazacyclododecane (DOTP), 1,4,7,10-tetraazacyclododecane-1,4,7,10-tetrakis(methylene methylphosphonic acid) (DOTMP), 1,4,7,10-tetraazacyclododecane-1,4,7,10-tetrakis(methylene phenylphosphonic acid) (DOTPP), N,N′-ethylenedi-L-cysteine, 1,4,7-triazacyclononane-1,4,7-triacetic acid (NOTA), 1,4,7-triazacyclononane (TACN), N,N′-Bis(2-hydroxy-5-(ethylene-beta-carboxy)benzyl)ethylenediamine N,N′-diacetic acid (HBED-CC), S-2-(4-isothiocyanatobenzyl)-1,4,7,10-tetraazacylododecane tetracetic acid (p-SCN-Bn-DOTA), MeO-DOTA-NCS, [(R)-2-Amino-3-(4-isothiocyanatophenyl)propyl]-trans-(S,S)-cyclohexane-1,2-diamine-pentaacetic acid (CHX-A″-DTPA-NCS), 2-[4-nitrobenzyl]-1,4,7,10,13-pentaazacyclopentadecane-N,N′,N″,N′″,N″″-pentaacetic acid (PEPA), 1,4,7,10,13,16-hexaazacyclooctadecane-N,N′,N″,N′″,N″″-hexaacetic acid (HEHA), desferrioxamine B (DFO), or derivatives thereof.

The chelating agent can be complexed with a radionuclide or metal ion. In some embodiments, the metal ion includes at least one of a radioactive isotope of Ga, I, In, Y, Lu, Bi, Ac, Re, In, Th, Tc, Tl, Tb, Zr, Cu, Rb, At, Pb, Gd, Sm, or Sr. In other embodiments, the metal ion includes at least one of ¹⁸⁶Re, ¹⁸⁸Re, ^{99m}Tc, ¹⁵³Gd, ¹¹¹In, ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹Tb, ¹⁶¹Tb, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th.

Fluorescent labeling agents or infrared agents include those known to the art, many of which are commonly commercially available, for example, fluorophores, such as ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED, BODIPY TR-X, ALEXA 594, BODIPY 630/650-X, PULSAR 650, BODIPY 630/665-X, TEXAS RED, ALEXA 647, TideFluor, ICG, IR700, IR800, and QUASAR 670. Fluorescent labeling agents can include other known fluorophores, or proteins known to the art, for example, green fluorescent protein.

In other embodiments, the detectable moiety includes a fluorescent dye. Exemplary Fluorescent dyes include fluorescein isothiocyanate, cyanines such as Cy5, Cy5.5, Cy7.5, and analogs thereof (e.g., sulfo-Cyanine 5 NHS ester and Cy5.5 maleimide). See also Handbook of Fluorescent Probes and Research Chemicals, 6^{th} Ed., Molecular Probes, Inc., Eugene Oreg, which is incorporated herein by reference.

In some embodiments, the fluorescence labeling agent can include the cyanine monosuccinimidyl ester, CY5.5. In certain embodiments, the near-infrared (NIR) labeling agent can include IRDye700DX (IR700). In some embodiments, a CY5.5 fluorescence labeling agent or IR700 NIR labeling agent can be coupled to the amine group of lysine linked to the PSMA ligand via a carbon spacer arm linker. A carbon spacer arm can include a standard (C6) or a longer spacer arm, such as a (C12) spacer arm. For example, the carbon spacer arm can be a C6, C7, C8, C9, C10, C11, or C12 spacer arm. In certain embodiments, the C6 carbon spacer arm is a 6-Aminohexanoic acid (Ahx) spacer arm.

The detectable moiety can further include a near infrared imaging group. Near infrared imaging groups are disclosed in, for example, Tetrahedron Letters 49(2008) 3395-3399; Angew. Chem. Int. Ed. 2007, 46, 8998-9001; Anal. Chem. 2000, 72, 5907; Nature Biotechnology vol 23, 577-583; Eur Radiol(2003) 13: 195-208; and Cancer 67: 1991 2529-2537, which are herein incorporated by reference in their entirety. Applications may include the use of a NIRF (near infra-red) imaging scanner. In one example, the NIRF scanner may be handheld. In another example, the NIRF scanner may be miniaturized and embedded in an apparatus (e.g., micro-machines, scalpel, neurosurgical cell removal device).

Quantum dots, e.g., semiconductor nanoparticles, can be employed in a molecular probe as described in Gao, et al “In vivo cancer targeting and imaging with semiconductor quantum dots”, Nature Biotechnology, 22, (8), 2004, 969-976, the entire teachings of which are incorporated herein by reference. The disclosed PSMA ligangs can be coupled to the quantum dots, administered to a subject or a sample, and the subject/sample examined by fluorescence spectroscopy or imaging to detect the labeled compound.

In certain embodiments, a detectable moiety includes a MRI contrast agent. MRI relies upon changes in magnetic dipoles to perform detailed anatomic imaging and functional studies. MRI can employ dynamic quantitative T₁ mapping as an imaging method to measure the longitudinal relaxation time, the Ti relaxation time, of protons in a magnetic field after excitation by a radiofrequency pulse. Ti relaxation times can in turn be used to calculate the concentration of a molecular probe in a region of interest, thereby allowing the retention or clearance of an agent to be quantified. In this context, retention is a measure of molecular contrast agent binding.

Numerous magnetic resonance imaging (MRI) contrast agents are known to the art, for example, positive contrast agents and negative contrast agents. The disclosed targeting peptides can be coupled to the MRI agents, administered to a subject or a sample, and the subject/sample examined by MRI or imaging to detect the labeled compound. Positive contrast agents (typically appearing predominantly bright on MRI) can include typically small molecular weight organic compounds that chelate or contain an active element having unpaired outer shell electron spins, e.g., gadolinium, manganese, iron oxide, or the like. Typical contrast agents include macrocycle-structured gadolinium(III)chelates, such as gadoterate meglumine (gadoteric acid), gadopentetate dimeglumine, gadoteridol, mangafodipir trisodium, gadodiamide, and others known to the art. In certain embodiments, the detectable moiety includes gadoterate meglumine. Negative contrast agents (typically appearing predominantly dark on MRI) can include small particulate aggregates comprised of superparamagnetic materials, for example, particles of superparamagnetic iron oxide (SPIO). Negative contrast agents can also include compounds that lack the hydrogen atoms associated with the signal in MRI imaging, for example, perfluorocarbons (perfluorochemicals).

In some embodiments, a PSMA targeted conjugate compound that is coupled to a fluorescence labeling agent, near-infrared or infrared agent can have the following general formula:

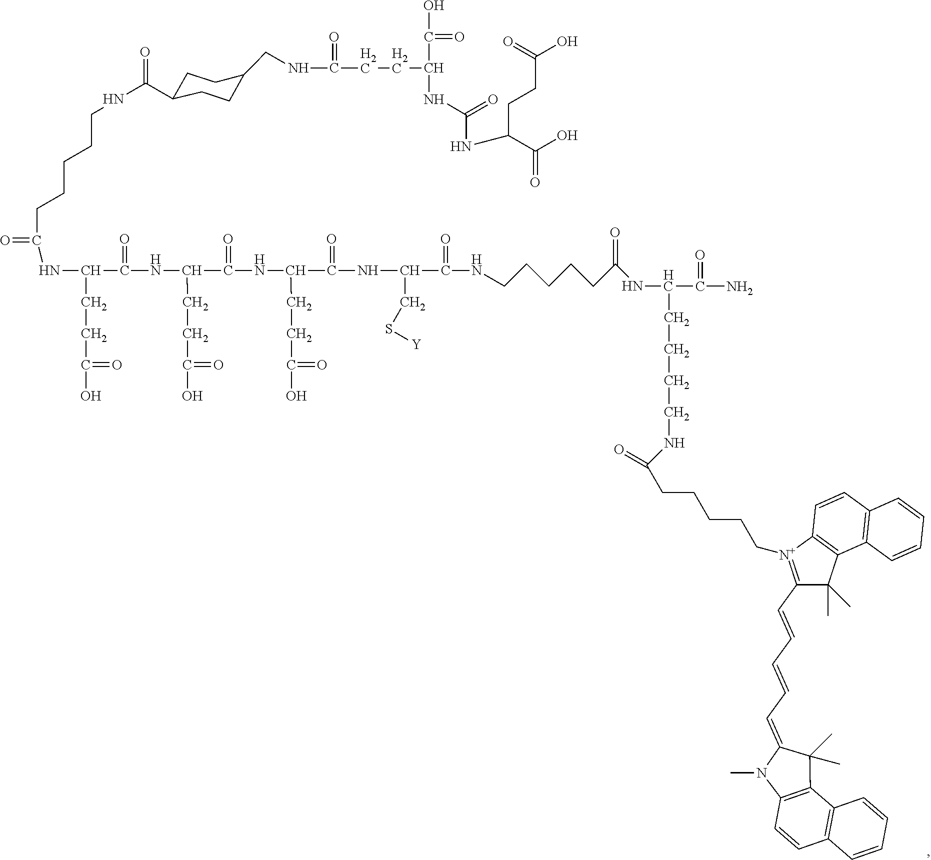

wherein Y is selected from at least one of a detectable moiety, therapeutic agent, or theranostic agent that is directly or indirectly linked to thiol group. This PSMA targeted conjugate compound can be prepared by linking the cysteine residue of the PSMA ligand to a lysine residue via a C6 linker, which then allows for coupling a detectable moiety, therapeutic agent, or theranostic agent, for example, the fluorescence labeling agent, CY5.5 to the amine on the lysine amino acid.

In some embodiments, a PSMA targeted conjugate compound that is coupled to a fluorescence labeling agent and a therapeutic agent can have the formula:

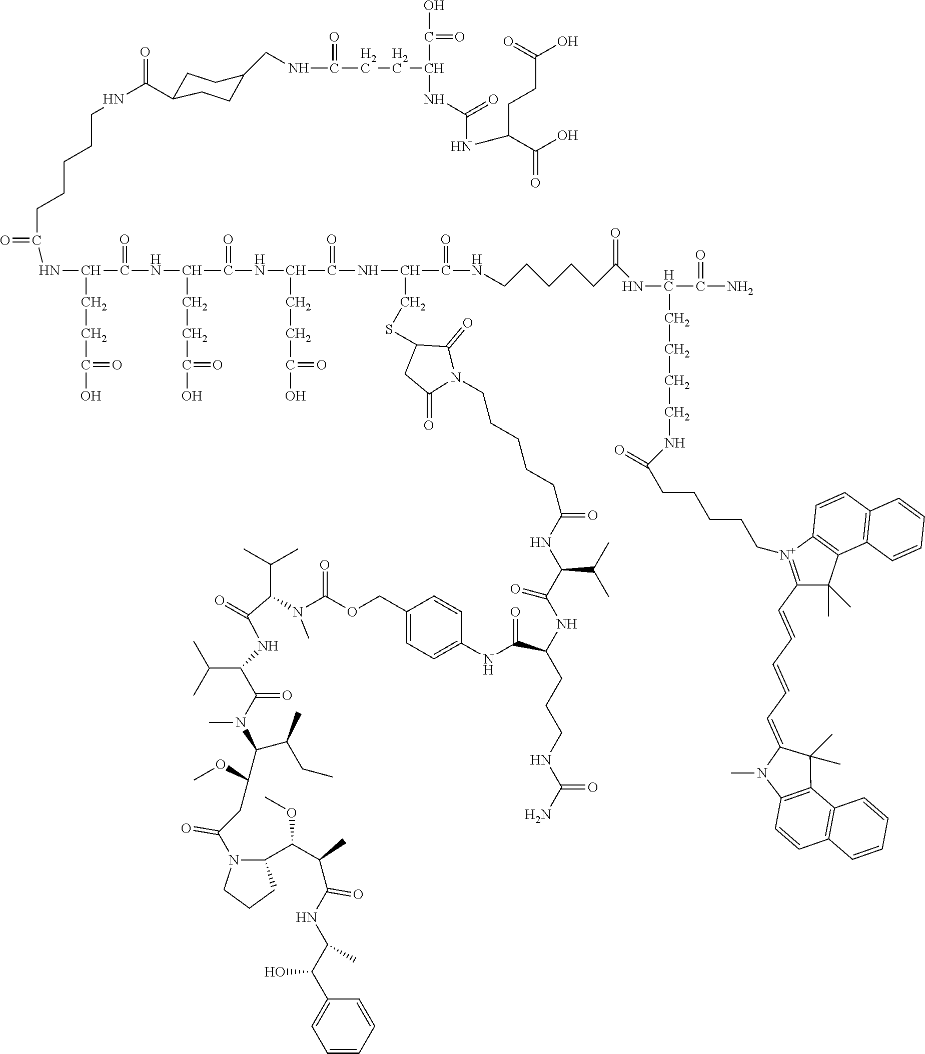 [SMILES: CC[C@H](C)[C@@H]([C@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SCC(NC(=O)C(CCC(=O)O)NC(=O)C(CCC(=O)O)NC(=O)C(CCC(=O)O)NC(=O)CCCCCNC(=O)C3CCC(CNC(=O)CCC(NC(=O)NC(CCC(=O)O)C(=O)O)C(=O)O)CC3)C(=O)NCCCCCC(=O)NC(CCCCNC(=O)CCCCC[N+]3=C(/C=C/C=C/C=C4/N(C)c5ccc6ccccc6c5C4(C)C)C(C)(C)c4c3ccc3ccccc43)C(N)=O)C2=O)C(C)C)cc1)C(C)C]

or a pharmaceutically acceptable salt thereof.

In some embodiments, a PSMA targeted conjugate compound that is coupled to a fluorescence labeling agent, near-infrared agent or infrared agent can have the following general formula:

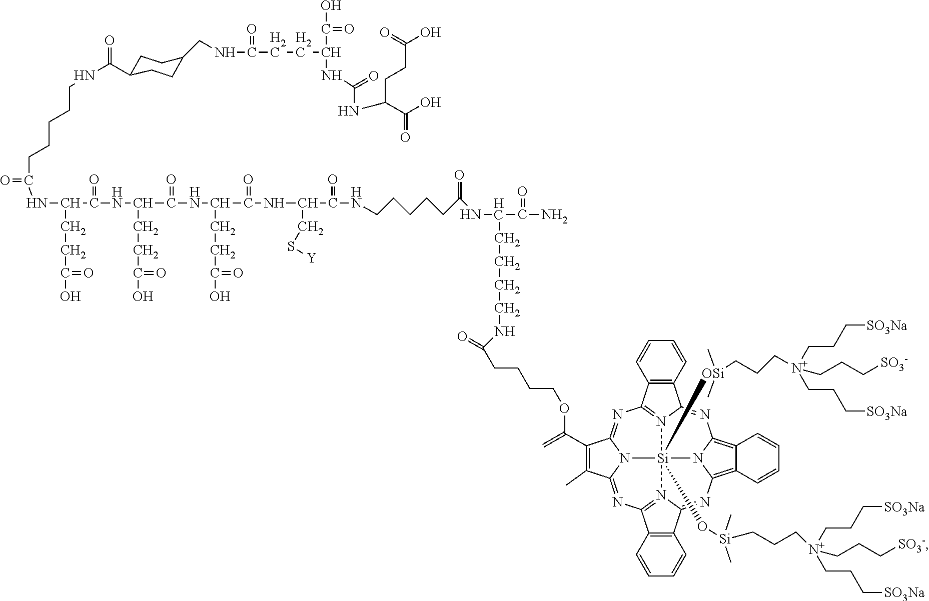

wherein Y is a therapeutic agent, such as an anti-cancer agent or chemotherapeutic, that is directly or indirectly linked to the thiol group. This PSMA targeted conjugate compound can be prepared by linking the cysteine residue of the PSMA ligand to a lysine residue via a C6 linker, which then allows for coupling the near-infrared (NIR) labeling agent, IR700 to the amine on the lysine amino acid.

In some embodiments, a PSMA targeted conjugate compound that is coupled to a near-infrared labeling agent IR700 and a therapeutic agent can have the formula:

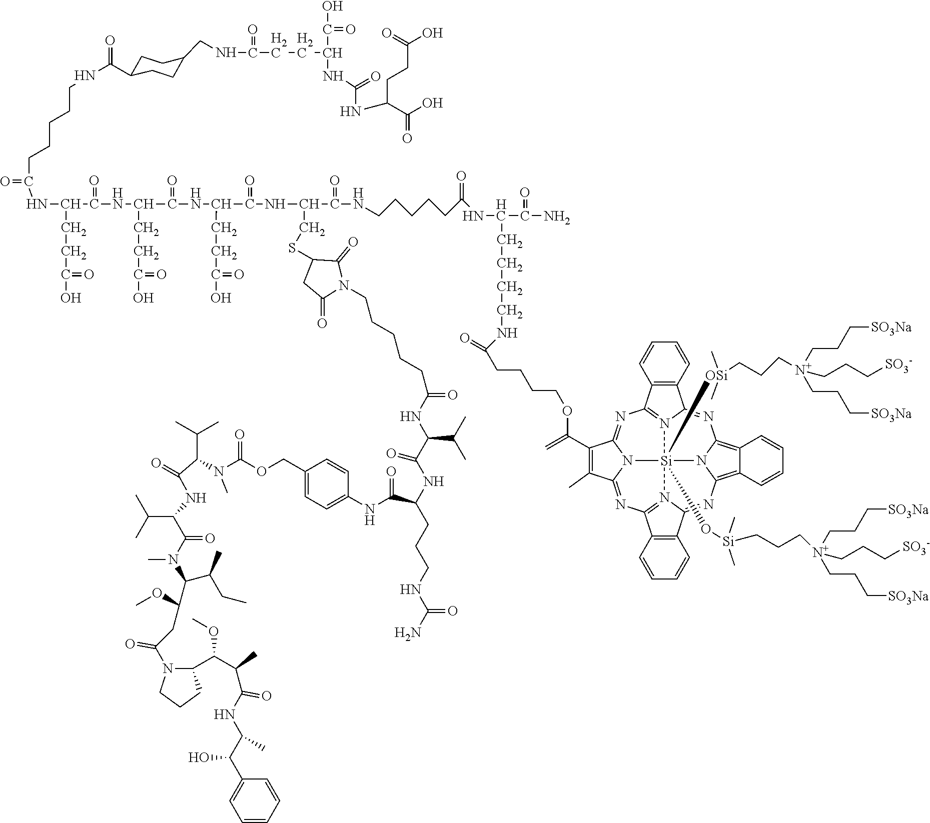

or a pharmaceutically acceptable salt thereof.

In some embodiments, a PSMA targeted conjugate compound that is coupled to an anti-cancer agent, such as MMAE, and a chelating agent optionally complexed with a metal ion can have the following formula:

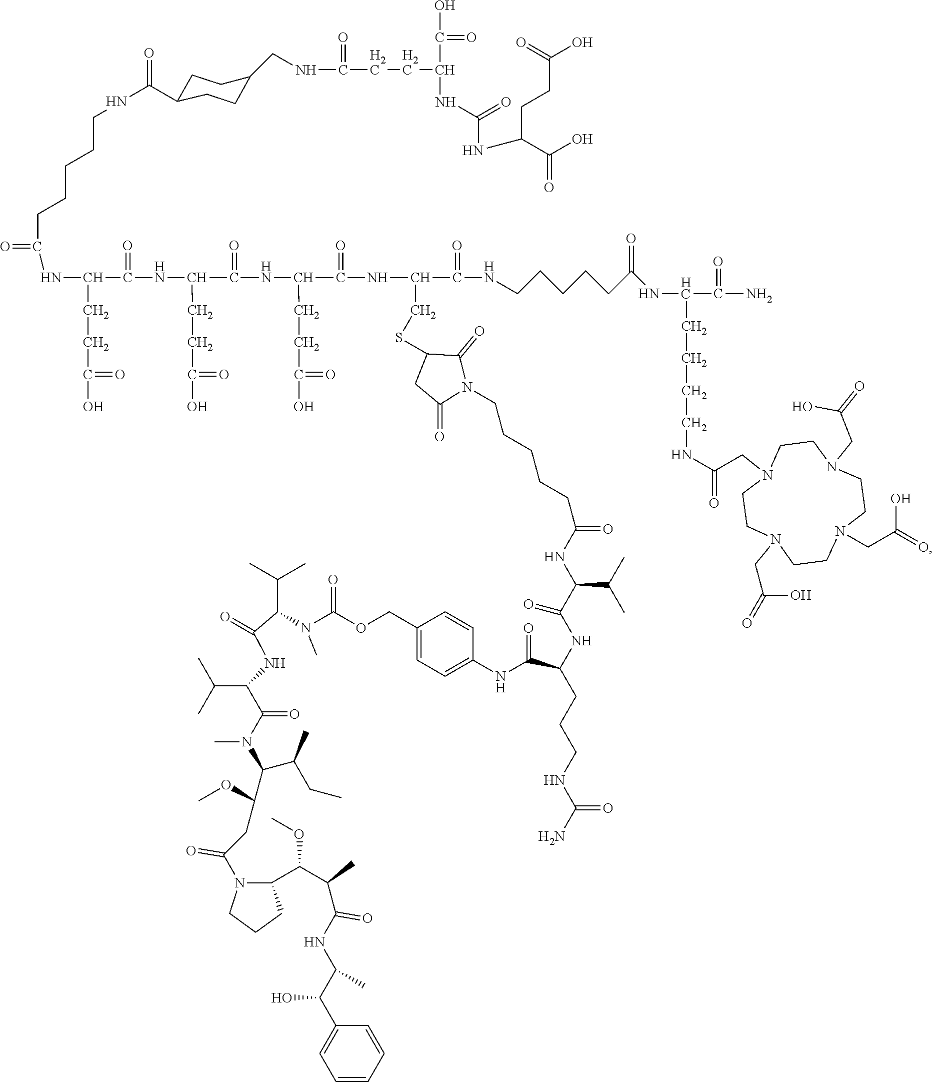 [SMILES: CC[C@H](C)[C@@H]([C@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SCC(NC(=O)C(CCC(=O)O)NC(=O)C(CCC(=O)O)NC(=O)C(CCC(=O)O)NC(=O)CCCCCNC(=O)C3CCC(CNC(=O)CCC(NC(=O)NC(CCC(=O)O)C(=O)O)C(=O)O)CC3)C(=O)NCCCCCC(=O)NC(CCCCNC(=O)CN3CCN(CC(=O)O)CCN(CC(=O)O)CCN(CC(=O)O)CC3)C(N)=O)C2=O)C(C)C)cc1)C(C)C]

or a pharmaceutically acceptable salt thereof. The metal ion complexed with the chelating agent can include at least one of a radioactive isotope of Ga, I, In, Y, Lu, Bi, Ac, Re, In, Th, Tc, Tl, Zr, Cu, R^{b}, At, Pb, Gd, Sm, or Sr. In other embodiments, the metal ion includes at least one of ¹⁸⁶Re, ¹⁸⁸Re, ⁹⁹mTc ¹⁵³Gd, ¹¹¹In, ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹Tb, ¹⁶¹Tb, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³Th.

In some aspects, the PSMA ligands coupled to a detectable moiety described herein may be used in conjunction with non-invasive imaging techniques for in vivo imaging, such as magnetic resonance spectroscopy (MRS) or imaging (MRI), or gamma imaging, such as positron emission tomography (PET) or single-photon emission computed tomography (SPECT), to determine the location or distribution of cancer cells. The term “in vivo imaging” refers to any method, which permits the detection of a labeled PSMA targeted conjugate compound, as described above. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of molecular probe along with a large excess of unlabeled, but otherwise chemically identical compound.

For purposes of in vivo imaging, the type of detection instrument available is a major factor in selecting a given detectable moiety. For instance, the type of instrument used will guide the selection of the stable isotope. The half-life should be long enough so that it is still detectable at the time of maximum uptake by the target, but short enough so that the host does not sustain deleterious effects.

The PSMA targeted conjugate compounds coupled to a detectable moiety described herein can be administered to the subject by, for example, systemic, topical, and/or parenteral methods of administration. These methods include, e.g., injection, infusion, deposition, implantation, or topical administration, or any other method of administration where access to the tissue desired. In one example, administration can be by intravenous injection in the subject. Single or multiple administrations of the probe can be given. “Administered”, as used herein, means provision or delivery is in an amount(s) and for a period of time(s) effective to label cancer cells in the subject.

The PSMA targeted conjugate compounds coupled to a detectable moiety described herein can be administered to a subject in a detectable quantity of a pharmaceutical composition containing the PSMA targeted conjugate compounds coupled to a detectable moiety described herein or a pharmaceutically acceptable water-soluble salt thereof, to a patient. A “detectable quantity” means that the amount of the detectable compound that is administered is sufficient to enable detection of binding of the compound to the cancer cells. An “imaging effective quantity” means that the amount of the detectable compound that is administered is sufficient to enable imaging of binding of the compound to the cancer cells.

The PSMA targeted conjugate compounds coupled to a detectable moiety described herein administered to a subject can be used to determine the presence, location, and/or distribution of cancer cells, i.e., PSMA expressing cancer cells or PSMA expressing neovaculature of the cancer cells, in an organ or body area of a patient. The presence, location, and/or distribution of the PSMA ligands coupled to a detectable moiety in the animal's tissue, e.g., brain tissue, can be visualized (e.g., with an in vivo imaging modality described above). “Distribution” as used herein is the spatial property of being scattered about over an area or volume. In this case, “the distribution of cancer cells” is the spatial property of cancer cells being scattered about over an area or volume included in the animal's tissue, e.g., prostate tissue. The distribution of the PSMA targeted conjugate compounds coupled to a detectable moiety may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject.

In some embodiments, the PSMA targeted conjugate compounds coupled to a detectable moiety may be administered to a subject to assess the distribution prostate cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of prostate on the tumor margin that are outside the tumor edge that appear to be grossly normal but are infiltrated by dispersing tumor cells upon histological examination.

The PSMA targeted conjugate compounds coupled to a detectable moiety can be used in intra-operative imaging techniques to guide surgical resection and eliminate the “educated guess” of the location of the tumor by the surgeon. Previous studies have determined that more extensive surgical resection improves patient survival. Thus, PSMA targeted conjugate compounds coupled to a detectable moiety that function as diagnostic molecular imaging agents have the potential to increase patient survival rates. PSMA-targeted conjugates are easily detectable at real-time imaging exposures (i.e., at <67 ms) and thus capable of being used for real-time image-guided surgery (IGS) during urological surgery. Thus, the compositions described herein for use in IGS can increase patient survival rates especially when including a therapeutic agent and/or when combined with subsequent photodynamic therapy (PDT) in accordance with a method described herein.

In some embodiments, IGS can be performed real-time using an in vivo imaging system, such as an intraoperative near-infrared fluorescence imaging system. In certain embodiments, image guided surgery can be performed using a FLARE (Fluorescence-Assisted Resection and Exploration) intraoperative NIR fluorescence imaging system where the targeted cancer is imaged at a wavelength of about 671 nm to about 705 nm. In an exemplary embodiment, the targeted PMSA expressing cancer is imaged using a PSMA conjugate compound coupled to a NIR-imaging agent during image guided surgical resection at about 690 to about 700 nm.

Following surgical resection, residual PSMA expressing cancer cells that have penetrated beyond the resection site may revert to a proliferative state to produce a more aggressive recurrent tumor that continues to disperse into nonneoplastic tissue adjacent the resection site and beyond. A therapeutic method described herein can be used to minimize cancer metastasis after a surgical resection procedure targeting PSMA positive cancer cells and/or tumor tissue using PDT.

Therefore, a therapeutic method described herein further includes the step of irradiating the PSMA-targeted conjugate compounds coupled to a photosensitizing agent that are bound to and/or complexed with the PSMA expressing cancer cells remaining in, and/or adjacent to, the surgical site to induce the cytotoxic effects of a coupled photosensitizing agent, such as the NIR-imaging agent IR700, on residual cancer cells following surgical resection of the cancer.

Methods and photosensitizing agents for conducting PDT are known in the art. See for example Thierry Patrice. Photodynamic Therapy; Royal Society of Chemistry, 2004. PDT is a site specific treatment modality that requires the presence of a photosensitizer, light, and adequate amounts of molecular oxygen to destroy targeted tumors (Grossweiner, Li, The science of phototherapy. Springer: The Netherlands, 2005). Upon illumination, a photoactivated sensitizer transfers energy to molecular oxygen that leads to the generation of singlet oxygen (O²) and other reactive oxygen species (ROS), which initiate apoptosis and oxidative damage to cancer cells. Only the cells that are exposed simultaneously to the targeted PDT compound (which is non-toxic in the dark) and light are destroyed while surrounding healthy, non-targeted and nonirradiated cells are spared from photodamage. Furthermore, the fluorescence of the photosensitizing compound, such as IR700, coupled to the PSMA targeting moiety enable simultaneous diagnostic optical imaging that can be used to guide the PDT step of the method of treating cancer described herein.

PDT agent photosensitizer compounds for use in an agent described herein can include compounds that are excited by an appropriate light source to produce radicals and/or reactive oxygen species. Typically, when a sufficient amount of photosensitizer appears in diseased tissue (e.g., tumor tissue), the photosensitizer can be activated by exposure to light for a specified period. The light dose supplies sufficient energy to stimulate the photosensitizer, but not enough to damage neighboring healthy tissue. The radicals or reactive oxygen produced following photosensitizer excitation kill the target cells (e.g., cancer cells). In some embodiments, the targeted tissue can be locally illuminated. For example, light can be delivered to a photosensitizer via an argon or copper pumped dye laser coupled to an optical fiber, a double laser consisting of KTP (potassium titanyl phosphate)/YAG (yttrium aluminum garnet) medium, LED (light emitting diode), or a solid state laser.

PDT sensitizers for use as a theranostic or therapeutic agent can include a first generation photosensitizer (e.g., hematoporphyrin derivatives (HpDs) such as Photofrin (porfimer sodium), Photogem, Photosan-3 and the like). In some embodiments, PDT sensitizers can include second and third generation photosensitizers such as porphyrinoid derivatives and precursors. Porphyrinoid derivatives and precursors can include porphyrins and mettaloporphrins (e.g., meta-tetra(hydroxyphenyl)porphyrin (m-THPP), 5,10,15,20-tetrakis(4-sulfanatophenyl)-21H,23H-porphyrin (TPPS₄), and precursors to endogenous protoporphyrin IX (PpIX): 5-aminolevulinic acid (5-ALA, which has been used for photodynamic therapy (PDT) of gliomas with some success (Stummer, W. et al. J Neurooncol. 2008, 87(1):103-9.).), IR700 dye, methyl aminolevulinate (MAL), hexaminolevulinate (HAL)), chlorins (e.g., benzoporphyrin derivative monoacid ring A (BPD-MA), meta-tetra(hydroxyphenyl)chlorin (m-THPC), N-aspartyl chlorine6 (NPe6), and tin ethyl etiopurpurin (SnET2)), pheophorbides (e.g., 2-(1-hexyloxyethyl)-2-devinyl pyropheophorbide (HPPH)), bacteriopheophorbides (e.g., bacteriochlorphyll a, WST09 and WST11), Texaphyrins (e.g., motexafin lutetium (Lu-Tex)), and phthalocyanines (PCs) (e.g., aluminum phthalocyanine tetrasulfonate (AlPcS4) and silicon phthalocyanine (Pc4)). In some embodiments, the PDT sensitizer can include cationic zinc ethynylphenyl porphyrin. Although porphyrinoid structures comprise a majority of photosensitizers, several non-porphyrin chromogens exhibit photodynamic activity. These compounds include anthraquinones, phenothiazines, xanthenes, cyanines, and curcuminoids.

In some embodiments, a theranostic agent or therapeutic agent described herein can include a phthalocyanine compound. Phthalocyanines, hereinafter also abbreviated as “Pcs”, are a group of photosensitizer compounds having the phthalocyanine ring system. Phthalocyanines are azaporphyrins consisting of four benzoindole groups connected by nitrogen bridges in a 16-membered ring of alternating carbon and nitrogen atoms (i.e., C₃₂H₁₆N₈) which form stable chelates with metal and metalloid cations. In these compounds, the ring center is occupied by a metal ion (either a diamagnetic or a paramagnetic ion) that may, depending on the ion, carry one or two ligands. In addition, the ring periphery may be either unsubstituted or substituted. Phthalocyanines strongly absorb clinically useful red or near IR radiation with absorption peaks falling between about 600 and 810 nm, which potentially allows deep tissue penetration by the light. The synthesis and use of a wide variety of phthalocyanines in photodynamic therapy is described in International Publication WO 2005/099689.

In some embodiments, the phthalocyanine compound is Pc4. Pc4 is relatively photostable and virtually non-toxic. In some embodiments, the phthalocyanine compound is an analog of the PDT photosensitizing drug Pc4 found to be effective in targeted bioimaging and targeted PDT of cancer in a subject, see for example, U.S. Pat. No. 9,889,199, the contents of which are hereby incorporated by reference. In some embodiments, the Pc4 analog can include Pc413.

In certain embodiments, anear-infrared (NIR) labeling agent, such as IRDye700DX (IR700), can be used as the photosensitizing agent.

Following administration and detection/localization of PSMA-targeted conjugate compounds, the targeted cancer cells can be exposed to therapeutic amount of light that causes cancer cell damage and/or suppression of cancer cell growth. The light, which is capable of activating the PDT therapeutic agent can delivered to the targeted cancer cells, using for example, semiconductor laser, dye laser, optical parametric oscillator or the like. It will be appreciated that any source light can be used as long as the light excites the phthalocyanine compound bound or complexed with a PSMA expressing cancer cell.

In some embodiments, the surgical resection site can be irradiated using visible laser diodes to photoactivate the photosensitizing compound coupled to the PSMA targeting moiety. For example, in some embodiments, the surgical resection site can be irradiated using visible laser diodes emitting at 672 nm. In certain embodiments, the surgical resection site can be irradiated with an amount of radiation effective to inhibit tumor recurrence in the subject. In an exemplary embodiment, the PDT step of the method of treating cancer described herein can include irradiating the resection site bed with a 672 nm laser for 12.5 minutes with total radiant exposure of 75 J/cm².

In some embodiments, where the sensitizing agent coupled to the PSMA targeting moiety is IR700, the surgical resection site can be irradiated using visible laser diodes emitting at 690 nm. It is contemplated that the absorption peak of IR700 at the near infra-red permits increased light penetration across tissues. Photoactivation of IR700 specifically kills PSMA-expressing tumor cells while sparing the tumor microenvironment as well as the surrounding healthy tissues.

Following the PDT step of a method of treating cancer described herein, the resection site of a subject can be further imaged and irradiated after a period of time(s) to detect and ablate residual PSMA expressing cancer cells that may have survived previous irradiation. This optional step may or may not include additional administration of a pharmaceutical composition including a PSMA targeted conjugate compound described herein.

The PSMA targeted conjugate compounds can be used to deliver a therapeutic anti-cancer agent to a PSMA expressing cancer cell of a subject or mammal. Therefore, some embodiments relate to the administration of a PSMA targeted conjugate compound coupled to an anticancer agent to a subject having or suspected of having cancer, such as a PSMA expressing cancer for the treatment of cancer. In particular embodiments, a PSMA targeted conjugate compounds coupled to an anticancer agent can be administered to a subject having prostate cancer.

The PSMA targeted conjugate compounds can target and transiently interact with, bind to, and/or couple with a cancer cell, such as a prostate cancer cell, and once interacting with, bound to, or coupled to the targeted cell or tissue advantageously facilitate delivery of a therapeutic agent within cell by, for example, receptor mediated endocytosis. Without being bound by theory, where the PSMA targeted conjugate compound includes a therapeutic agent, it is believed that once delivered to a PSMA expressing cell, endosomal conditions can release the therapeutic agent, e.g., doxorubicin, where it travels to the nucleus, binds to DNA and exerts anti-proliferative effects.

Examples of anticancer agents that can be directly or indirectly coupled to a PSMA targeting conjugate compound as described herein can include, but are not limited to chemotherapeutic agents and other agents including Taxol, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon α-2a; interferon α-2b; interferon α-n1; interferon α-n3; interferon β-I a; interferon γ-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; silicon phthalocyanine (PC4) sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans (GAGs); tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Still other examples of anti-cancer agents can include the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Arnad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCl), Resverastatin phosphate sodium, BPR-OY-007 (National Health Research Institutes), and SSR-250411 (Sanofi).

Other examples of anti-cancer agents include alkylating agents, such as nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.), antimetabolites, such as folic acid analog (e.g., methotrexate), or pyrimidine analogs (e.g., fluorouracil, floxouridine, Cytarabine), purine analogs (e.g., mercaptopurine, thioguanine, pentostatin, vinca alkaloids (e.g., vinblastin, vincristine), epipodophyllotoxins (e.g., etoposide, teniposide), platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, amino glutethimide).

In particular embodiments, the anti-cancer agent is coupled to the PSMA targeted conjugate compound. In certain embodiments, the anti-cancer agent coupled to a PSMA targeted conjugate compound is selected from doxorubicin or an ant-mitotic agent or monomethyl auristatin E (MMAE).

In an embodiment, a PSMA targeted conjugate compound that is coupled to a therapeutic anti-cancer agent can have the formula:

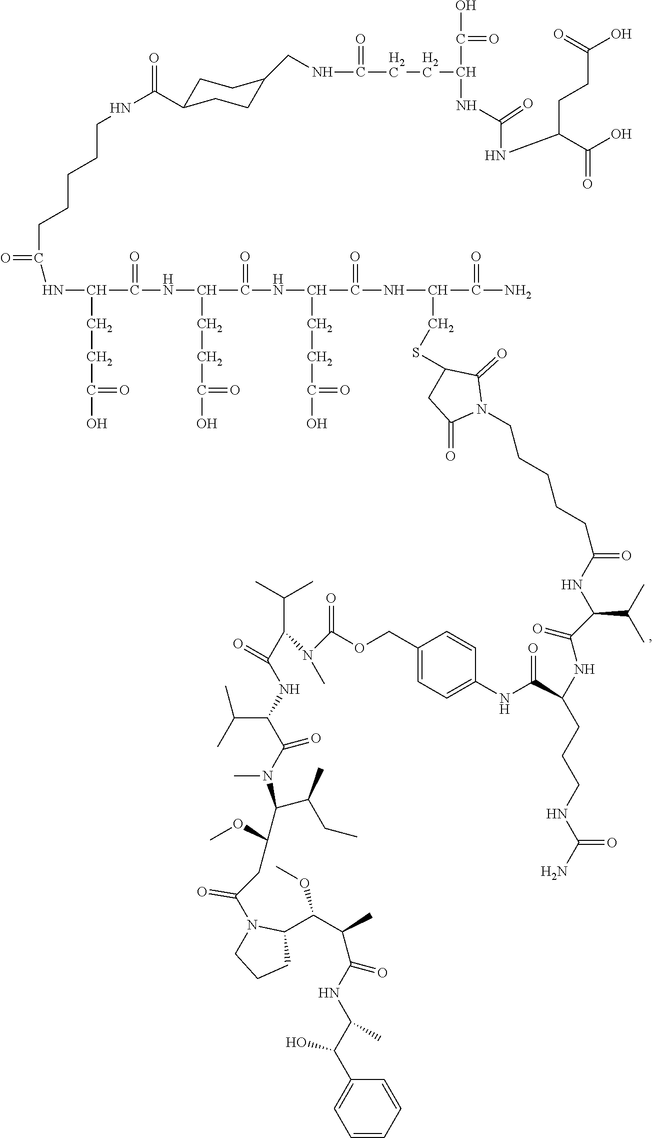 [SMILES: CC[C@H](C)[C@@H]([C@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SCC(NC(=O)C(CCC(=O)O)NC(=O)C(CCC(=O)O)NC(=O)C(CCC(=O)O)NC(=O)CCCCCNC(=O)C3CCC(CNC(=O)CCC(NC(=O)NC(CCC(=O)O)C(=O)O)C(=O)O)CC3)C(N)=O)C2=O)C(C)C)cc1)C(C)C]

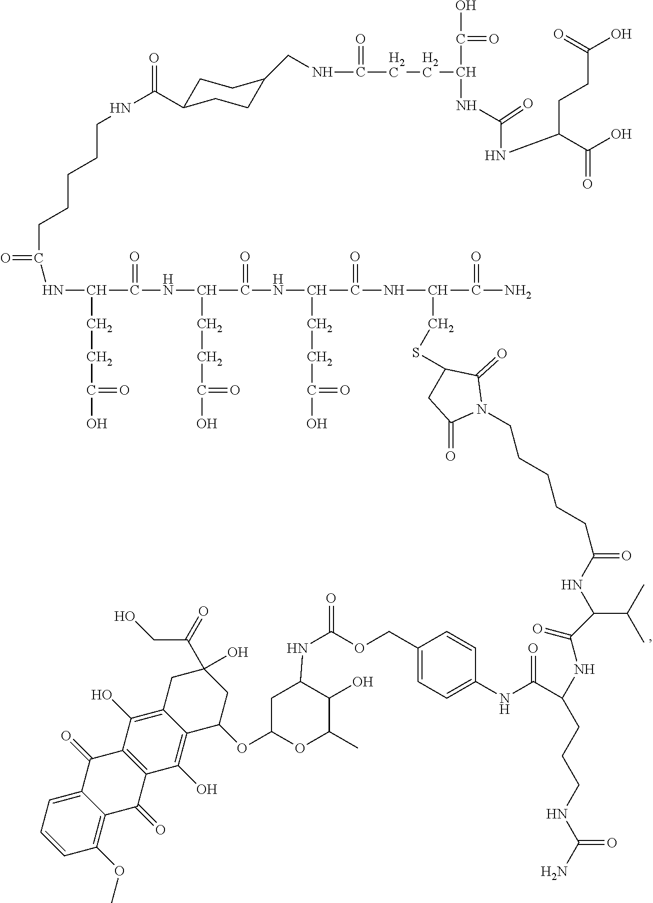 [SMILES: COc1cccc2c1C(=O)c1c(O)c3c(c(O)c1C2=O)CC(O)(C(=O)CO)CC3OC1CC(NC(=O)OCc2ccc(NC(=O)C(CCCNC(N)=O)NC(=O)C(NC(=O)CCCCCN3C(=O)CC(SCC(NC(=O)C(CCC(=O)O)NC(=O)C(CCC(=O)O)NC(=O)C(CCC(=O)O)NC(=O)CCCCCNC(=O)C4CCC(CNC(=O)CCC(NC(=O)NC(CCC(=O)O)C(=O)O)C(=O)O)CC4)C(N)=O)C3=O)C(C)C)cc2)C(O)C(C)O1]

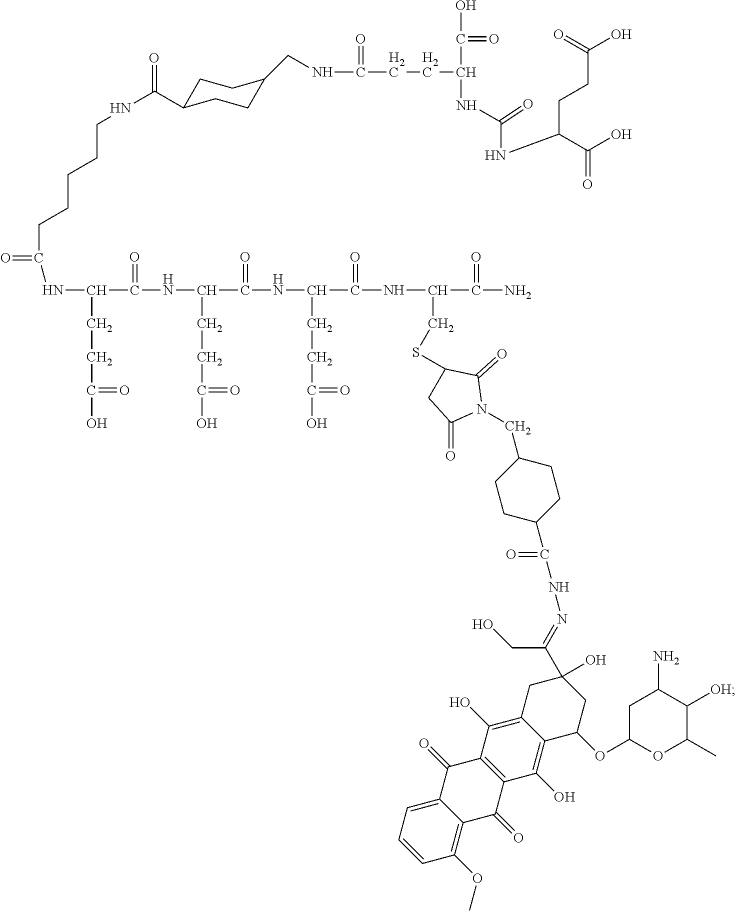 [SMILES: COc1cccc2c1C(=O)c1c(O)c3c(c(O)c1C2=O)CC(O)(/C(CO)=N/NC(=O)C1CCC(CN2C(=O)CC(SCC(NC(=O)C(CCC(=O)O)NC(=O)C(CCC(=O)O)NC(=O)C(CCC(=O)O)NC(=O)CCCCCNC(=O)C4CCC(CNC(=O)CCC(NC(=O)NC(CCC(=O)O)C(=O)O)C(=O)O)CC4)C(N)=O)C2=O)CC1)CC3OC1CC(N)C(O)C(C)O1]

or pharmaceutical salts thereof.

In some embodiments, PSMA targeted conjugate compounds can be coupled to both a therapeutic agent and a NIR imaging agent to provide targeted delivery of the therapeutic agent, such as an anti-cancer agent, while also allowing for tumor imaging and targeted photodynamic therapy.

In an embodiment, a PSMA targeted conjugate compound can be coupled to the anti-cancer agent MMAE via a MC-VC-PABC protease cleavable linker and coupled to the near-infrared labeling agent or photosensitizer IR700 (i.e., a PSMA-1-VcMMAE-IR700 conjugate compound) and have the formula:

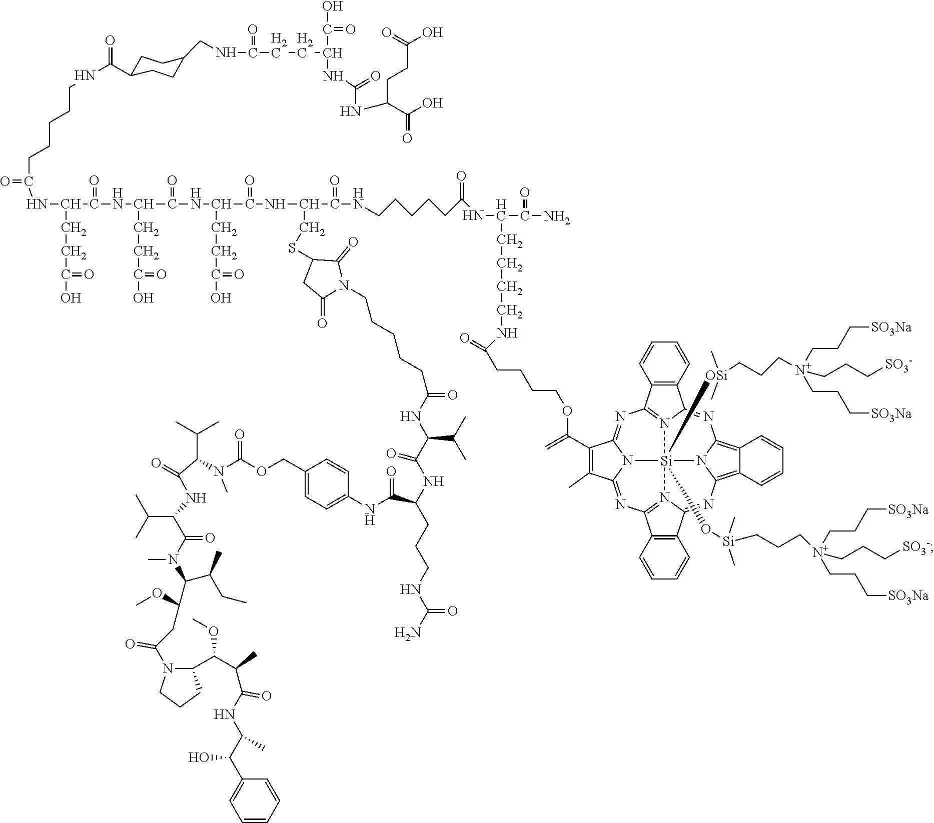

or a pharmaceutical salt thereof.

In an embodiment, PSMA-1-VcMMAE-IR700 conjugate compounds administered to subject can provide for targeted delivery of the MMAE chemotherapeutic agent to PSMA+prostate cancer cells while the NIR imaging agent IR700 coupled to PSMA-1 allows for image guidance during prostate tumor resection and subsequent targeted PDT to eliminate unresectable or remaining cancer cells.

In another embodiment, a PSMA targeted conjugate compound can be coupled to the anti-cancer agent MMAE via a MC-VC-PABC protease cleavable linker and coupled to chelating agent optionally complexed metal ion. For example, the PSMA targeted conjugate compound including MMAE and a chelating agent optionally complexed with a metal ion can have the following formula:

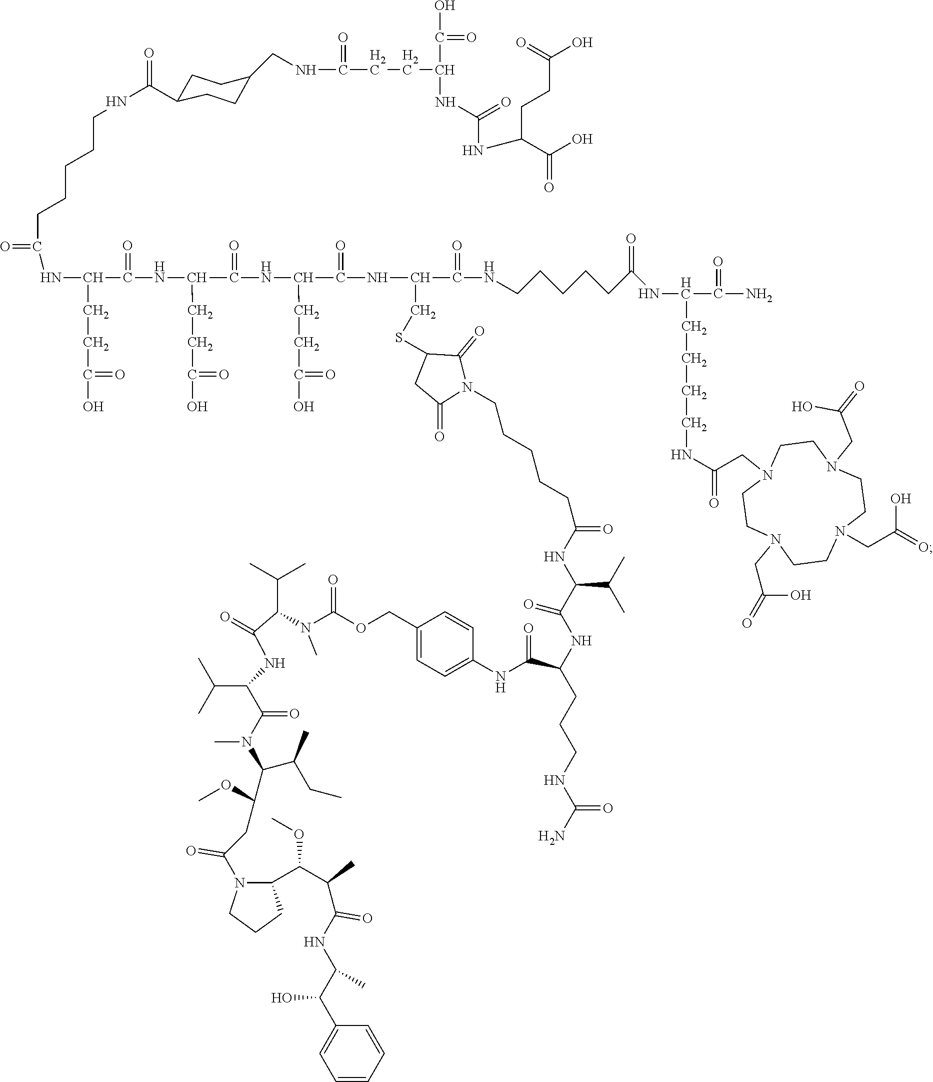 [SMILES: CC[C@H](C)[C@@H]([C@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SCC(NC(=O)C(CCC(=O)O)NC(=O)C(CCC(=O)O)NC(=O)C(CCC(=O)O)NC(=O)CCCCCNC(=O)C3CCC(CNC(=O)CCC(NC(=O)NC(CCC(=O)O)C(=O)O)C(=O)O)CC3)C(=O)NCCCCCC(=O)NC(CCCCNC(=O)CN3CCN(CC(=O)O)CCN(CC(=O)O)CCN(CC(=O)O)CC3)C(N)=O)C2=O)C(C)C)cc1)C(C)C]

or a pharmaceutically acceptable salt thereof. The metal ion complexed with the chelating agent can include at least one of a radioactive isotope of Ga, I, In, Y, Lu, Bi, Ac, Re, In, Th, Tc, Tl, Tb, Zr, Cu, R^{b}, At, Pb, Gd, Sm, or Sr. In other embodiments, the metal ion includes at least one of ¹⁸⁶Re, ¹⁸⁸Re, ^{99m}Tc, ¹⁵³Gd, ¹¹¹In, ⁶⁷Ga, ⁶⁸Ga, ²⁰¹Tl, ⁸²Rb, ⁶⁴Cu, ⁸⁹Zr, ⁹⁰Y, ¹⁷⁷Lu, T(tritium), ¹⁴⁹T, ¹⁶¹Tb, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th.

The PSMA targeting conjugate compounds can be administered alone as a monotherapy, or in conjunction with or in combination with one or more additional therapeutic agents. In some embodiments, a PSMA targeting conjugate compound described herein can be administered to the subject in combination with an additional anti-cancer agent. In a particular embodiment, a PSMA targeting conjugate compound coupled to doxorubicin as described herein can be administered to the subject in combination with the anti-cancer agent docetaxel.

It will be appreciated that additional detectable moieties, therapeutic agents, and/or theranostic agents administered to a subject need not be conjugated directly or indirectly to the PSMA ligand of the PSMA targeting compound and can optionally be provided in a pharmaceutical composition or preparation with the PSMA targeting conjugate compounds described herein or in a separate pharmaceutical composition.

The term “in conjunction with” or “in combination with” indicates that the PSMA targeting conjugate compound is administered at about the same time as the additional agent. The PSMA targeting conjugate compound can be administered to the subject in need thereof as part of a pharmaceutical composition comprising the compound and a pharmaceutically acceptable carrier or excipient and, optionally, one or more additional therapeutic agents. The compound and additional therapeutic agent can be components of separate pharmaceutical compositions, which can be mixed together prior to administration or administered separately. The PSMA targeting conjugate compound can, for example, be administered in a composition containing the additional therapeutic agent, and thereby, administered contemporaneously with the agent. Alternatively, the PSMA targeting conjugate compound can be administered contemporaneously, without mixing (e.g., by delivery of the compound on the intravenous line by which the compound is also administered, or vice versa). In another embodiment, the PSMA targeting conjugate compound can be administered separately (e.g., not admixed), but within a short timeframe (e.g., within 24 hours) of administration of the compound.

The disclosed PSMA targeting conjugate compounds and additional therapeutic agents, detectable moieties, and/or theranostic agents described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed compounds can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. In specific embodiments, oral, parenteral, or systemic administration (e.g., intravenous) are preferred modes of administration for treatment.

The methods described herein contemplate either single or multiple administrations, given either simultaneously or over an extended period of time. The PSMA targeting conjugate compound (or composition containing the compound) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the PSMA targeting conjugate compound is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the agent in the subject, the agent can be administered between, for example, once a day or once a week.

For example, the administration of the PSMA targeting conjugate compound described herein and/or the additional therapeutic agent can take place at least once on day 1, 2,3, 4,5, 6, 7, 8, 9,10,11,12,13, 14,15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

A disclosed PSMA targeting conjugate compound and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions, the active ingredient will ordinarily be present in an amount of about 0.5-95% by weight based on the total weight of the composition.

The amount of disclosed PSMA targeting conjugate compound and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the compound can also depend on the disease state or condition being treated along with the clinical factors and the route of administration of the compound.

For treating humans or animals, the amount of disclosed PSMA targeting conjugate compound and/or additional therapeutic agent administered (in milligrams of compound per kilograms of subject body weight) is generally from about 0.1 mg/kg to about 100 mg/kg, typically from about 1 mg/kg to about 50 mg/kg, or more typically from about 1 mg/kg to about 25 mg/kg. In a preferred embodiment, the effective amount of agent or PSMA targeting conjugate compound is about 1-10 mg/kg. In another preferred embodiment, the effective amount of agent or PSMA targeting conjugate compound is about 1-5 mg/kg. The effective amount for a subject can be varied (e.g., increased or decreased) over time, depending on the needs of the subject.

The term “unit dose” refers to a physically discrete unit suitable as unitary dosage for the subject, each unit containing a predetermined quantity of active material that can produce the desired therapeutic effect in association with the required diluent; e.g., carrier or vehicle. In addition to the ingredients particularly mentioned above, the formulations of the present invention may include other agents conventional in the art having regard to the type of formulation in question.

The disclosed PSMA targeting conjugate compound and/or additional therapeutic agent described herein can be administered to the subject in conjunction with an acceptable pharmaceutical carrier or diluent as part of a pharmaceutical composition for therapy. Formulation of the PSMA targeting conjugate compound to be administered will vary according to the route of administration selected (e.g., solution, emulsion, capsule, and the like). Suitable pharmaceutically acceptable carriers may contain inert ingredients that do not unduly inhibit the biological activity of the PSMA targeting conjugate compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The preparation of a pharmacological composition that contains active ingredients dissolved or dispersed therein is well understood in the art. Typically, such compositions are prepared as injectables as either liquid solutions or suspensions, however, solid forms suitable for solution, or suspensions, in liquid prior to use can also be prepared. Formulation will vary according to the route of administration selected (e.g., solution, emulsion, capsule).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, 0. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

The following examples are for the purpose of illustration only and are not intended to limit the scope of the claims, which are appended hereto.

### Example 1

In this Example, we used our PSMA ligand to selectively deliver the very potent microtubule disruption drug, monomethyl auristatin E (MMAE), to prostate cancer cells. Advantageously, using the PSMA ligand to selectively target a chemotherapeutic drug to PSMA was found to improve pharmacokinetics and enable more precise chemotherapy with lower side-effects. Using a prodrug strategy we developed a therapeutic small molecule, PSMA-1-VcMMAE, and a theranostic molecule, PSMA-1-VcMMAE-Cy5.5, in which the near-infrared dye Cy5.5 was incorporated in the structure to allow modification of pharmacokinetics and to follow tumor response to the treatment in mouse models. In heterotopic, orthotopic, and metastatic prostate cancer models in mice we found that both conjugates selectively and effectively inhibited PSMA-expressing tumor growth and prolonged animal survival with no obvious toxicity. PSMA-1-VcMMAE also had a much more favorable therapeutic index than either PSMA-1-VcMMAE-Cy5.5 or PSMA-ADC.

### Materials and Methods

### General

PSMA targeting peptide Glu-CO-Glu′-Amc-Ahx-Glu-Glu-Glu-Cys-C6-Lys (PSMA-1-Cys-C6-Lys) was synthesized by Fmoc chemistry. (S)-2-(3-((S)-5-amino-1-carboxypentyl)ureido)pentanedioic acid (Cys-CO-Glu) was custom made by Bachem Bioscience Inc. All the other chemicals were purchased from Sigma-Aldrich Inc. HPLC was performed on a Shimadzu HPLC system equipped with a SPD-20A prominence UV/visible detector and monitored at 220 nm and 254 nm. Preparative HPLC was achieved using Luna 5m C18(2) 100A column (250 mm×10 mm×5 mm; Phenomenex) at a flow rate of 2.5 m/min. Analytical HPLC was performed using an analytical Luna 5m C18(2) 100A column (250 mm×4.6 mm 5 mm; Phenomenex) at a flow rate of 0.8 mL/min. The gradient used was 10% to 90% acetonitrile against 0.1% trifluoroacetic acid over 30 minutes.

### Synthesis of PSMA-1-VcMMAE

PSMA-1-VcMMAE was synthesized as a prodrug by conjugating MMAE to the Cys residue in PSMA-1-Cys-C6-Lys via a maleimido caproyl valine-citruline (Vc) cathepsin-cleavable linker with a self immolative p-aminobenzyl carbamate (PABC) spacer. PSMA-1-Cys-C6-Lys (2.6 mg, 2 mol) was dissolved in 500 μL of phosphate buffer; then 2.2 μmol of Vc-MMAE (3.0 mg) (BOC Sci.) in 500 μL of DMF was added. The pH of the reaction mixture was adjusted to 8 by triethylamine. The mixture was stirred at room temperature for 1 hour, then went through HPLC to get purified PSMA-1-VcMMAE. Yield: 4.4 mg (85%). Retention time: 16.8 min. MS (C₁₂₃H₁₉₅N₂₃O₃₇S). calculated: 2618.3. found: 1310.7 ([M+2H]/2), 874.1 ([M+3H]/3).

### Synthesis of PSMA-1-VcMMAE-Cy5.5

PSMA-1-Cys-C6-Lys (2.6 mg, 2 mol) was dissolved in 500 μL of phosphate buffer; then 2.2 mol of Vc-MMAE (3.0 mg) (BOC Sci.) in 500 μL of DMF was added. The pH of the reaction mixture was adjusted to 8 by trimethylamine. After stirring at room temperature for 1 hour, Cy5.5 NHS ester 2.5 mol in 200 μL of DMF was added. The reaction mixture was stirred overnight, then the product was purified by semi-preparative HPLC. Yield: 3.1 mg (50%). Retention time: 18.8 min. MS (C₁₆₃H₂₃₆N₂₅O₃₈S). calculated: 3185.8. found: 1062.2 ([M+3H]/3), 796.9 ([M+4H]/4).

### Synthesis of PSMA-1-McMMAE-Cy5.5

PSMA-1-McMMAE-Cy5.5 was synthesized using a non-cleavable maleimido caproyl (Mc) linker. It was synthesized in the same way as PMA-1-MMAE-Cy5.5. Yield: 45%. Retention time: 20.9 min. MS (C₁₄₄H₂₀₉N₂₀O₃₃S). calculated: 2780.4. found: 1041.2 ([M+H+Na]/2), 942.2 ([M+H+2Na]/3).

### Cell Culture

Retrovirally transfected PSMA positive PC3pip cells and transfection control PC3flu cells were obtained from Dr. Michel Sadelain in 2000 (Laboratory of Gene Transfer and Gene Expression, Gene Transfer and Somatic Cell Engineering Facility, Memorial-Sloan Kettering Cancer Center, New York, NY). C4-2 cells were from ATCC. The cells were last sorted and checked by western blot in 2019; no genetic authentication was performed. Cells were maintained in RPMI1640 medium (Invitrogen) with 2 mM L-glutamine and 10% Fetal Bovine Serum at 37° C. and 5% CO₂ under a humidified atmosphere.

### Competitive Binding Assay

The assay was carried as previously reported by incubation PC3pip cells with different concentrations of drug conjugates in the presence of 10 nM N—[N—[(S)-1,3-dicarboxypropyl]carbamoyl]-S-[³H]-methyl-L-cysteine (³H-ZJ24) (GE Healthcare Life Sciences, Pittsburgh, PA). Radioactivity of cell pellet was counted by scintillation counter. The concentration required to inhibit 50% of binding was determined (IC₅₀) by GraphPad Prism 3.0.

### Enzymatic Cleavage of PSMA-1-MMAE-Cy5.5 by Cathepsin B

PSMA-1-VcMMAE-Cy5.5 or PSMA-1-McMMAE-Cy5.5 was added to 500 μL of activated human liver cathepsin B (Anthens Research and Technology) solution to a final concentration of 2 μM and incubated at 37° C. At different time intervals, 40 L of the solution was taken out into tubes loaded with 1 μL of 1 mM E64 protease inhibitor. The mixture was vortexed and then stored at −80° C. for future HPLC analysis to assess degradation of PSMA-1-VcMMAE-Cy5.5 or PSMA-1-McMMAE-Cy5.5. Studies were performed in triplicate.

### In Vitro Cytotoxicity Assay

Cells (1,000/well) were seeded in 96-well culture plates the day before treatment. Cells were incubated with various concentrations of drugs for 72 hours and cell viability evaluated by CCK-8 (Dojindo). The concentration required to reach 50% of cell proliferation was determined (IC₅₀) by GraphPad Prism 3.0.

### In Vitro Cellular Uptake Studies

PC3pip and PC3flu cells were seeded in p-Slide 8-Well Chamber Slide (ibidi GmbH) at 2,000 cells/well. When cells grew to 70% confluency, PSMA-1-VcMMAE-Cy5.5 or PSMA-1-McMMAE-Cy5.5 conjugations were added at 50 nM and incubated at 37° C. for 4 hours. Cells were washed with PBS and stained with DAPI and LysoOrange (Abcam) for 30 minutes at 37° and 5% CO₂, after which they were washed again with PBS and filled with fresh medium. Selectivity was determined by including 20-fold excess of the PSMA-1 ligand. The uptake and localization of the drug conjugates were visualized under a Leica HyVolution SP8 confocal microscope at 40×.

### Immunofluorescence Analysis of Alpha-Tubulin

Cells on coverslips at about 70% confluency were incubated with 5 nM of MMAE, PSMA-1-VcMMAE-Cy5.5 or PSMA-1-McMMAE-Cy5.5 for 24 hours. Cells were washed and fixed with 4% paraformaldehyde for 10 minutes, permeabilized with 0.1% Triton™ X-100 for 10 minutes, and blocked with 1% BSA for 1 hour at room temperature. Alpha-tubulin (B5-1-2) Alexa Fluor 488 Mouse Monoclonal Antibody (Invitrogen) was then added at 2 μg/mL in 0.1% BSA and incubated for 3 hours at room temperature. Cells were counterstained with DAPI, mounted with Fluor-Mount aqueous mounting solution, and observed under Leica DM4000B fluorescence microscopy at 40×.

### In Vivo NIR Imaging Studies

Under guidelines of the animal care and use committee at Case Western Reserve University (IACUC #150033) 6 to 8-week old male Balb/cathymic nude mice were implanted subcutaneously with 1×10⁶ of PC3flu and PC3pip on the left and right dorsum respectively. Mice received 40 nmol/kg of PSMA-1-VcMMAE-Cy5.5 or PSMA-1-McMMAE-Cy5.5 in PBS via tail vein injection when tumors reached 10 mm in diameter. Fluorescence imaging was performed using the Maestro In Vivo Imaging system (Perkin-Elmer). Multispectral images were unmixed into their component spectra (PSMA probes, autofluorescence, and background) to quantitate the average fluorescence intensity associated with the tumors using regions of interest (ROIs) around the tumors.

### Maximum Tolerated Dose (MTD)

Groups of three male mice received single injections of MMAE, PSMA-1-Vc-MMAE, PSMA-1-VcMMAE-Cy5.5 or PSMA-ADC via the tail vein to determine single-dose MTD. Mice were monitored daily for 14 days. The MTD was defined as the highest dose that did not cause serious overt toxicities or 20% weight loss in any of the animals.

### Heterotopic Survival Study

Male athymic nude mice were implanted subcutaneously with 1×10⁶ of PC3pip cells under the right dorsum of the mice. When tumor size reached approximately 100 mm³ (tumor volume=Length×width²/2), mice received drugs through tail vein injection. Mice were treated every four days with a total of five doses. Animals were weighed and tumor size measured every other day for 90 days. Cures were defined as no tumor present at end of the 90-day study. When tumors became too large or animals were moribund they were euthanized. Five mice were used in each group.

### Orthotopic Survival Study

Surgical orthotopic tumor implantation was performed as previously reported. Tumor growth/size was monitored using Siemens Acuson S2000 ultrasound scanner. When the tumors were at the appropriate size (5 mm as measured by ultrasound, approximately 2 weeks) animals were given PSMA-1-VcMMAE, PSMA-1-VcMMAE-Cy5.5, or PBS every four days with a total of 5 doses. Tumors were monitored every other day by ultrasound. Each group had five mice.

### Metastatic Survival Study

Male athymic nude mice were injected into the left ventricle of the heart with 1×10⁵ GFP labeled PC3pip cells to generate bone metastasis. One week later mice received 160 nmol/kg PSMA-1-VcMMAE-Cy5.5 or PBS every 4 days with a total of 5 doses and the progression of disease was monitored by GFP imaging. Five mice were used in each group.

### Statistics

Student t-test was used to compare inter-group differences. Kaplan-Meier survival data were analyzed by SAS9.4 using log-rank tests. A p value <0.05 was considered statistically significant for all comparisons.

### Results

### A Prodrug Strategy is Crucial for Antitumor Activity

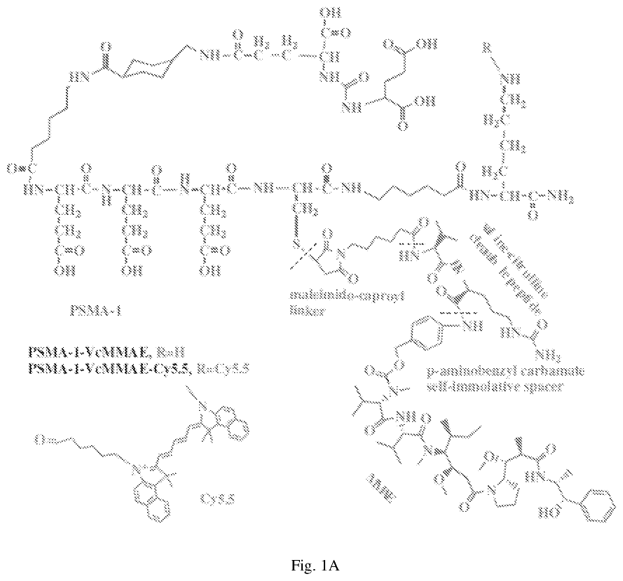

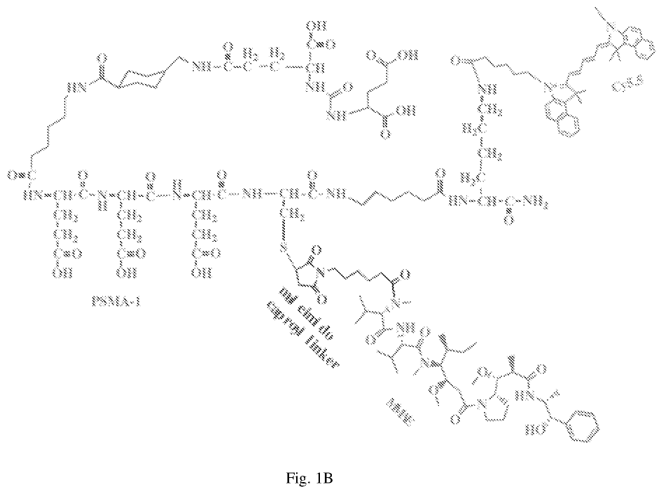

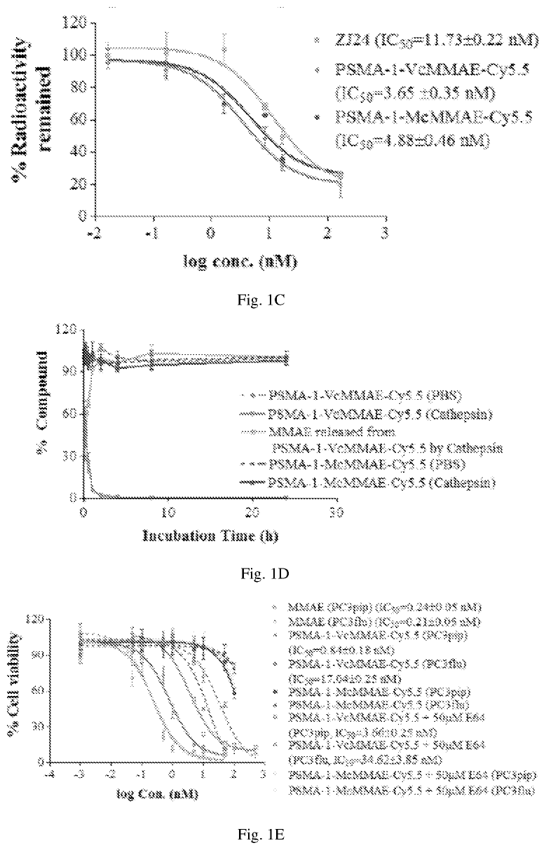

We initially synthesized two PSMA-targeted MMAE-Cy5.5 conjugates, one with a cathepsin-cleavable linker, PSMA-1-VcMMAE-Cy5.5, and the other with a non-cleavable linker, PSMA-1-McMMAE-Cy5.5, (FIGS. 1A-B) and characterized the antitumor activity of the small molecule-drug conjugates.

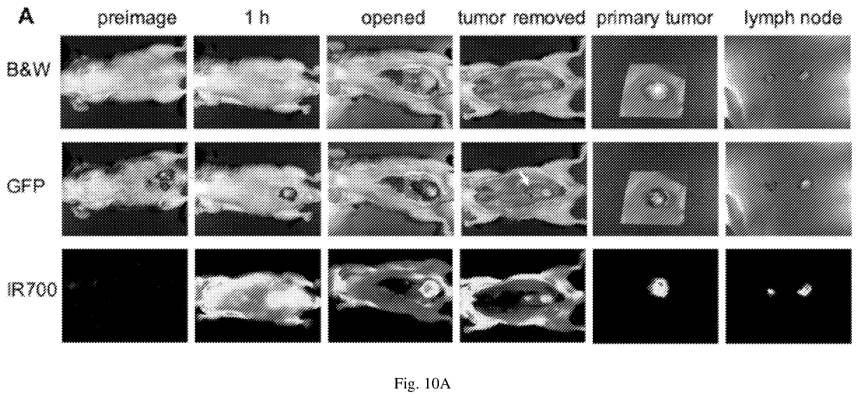

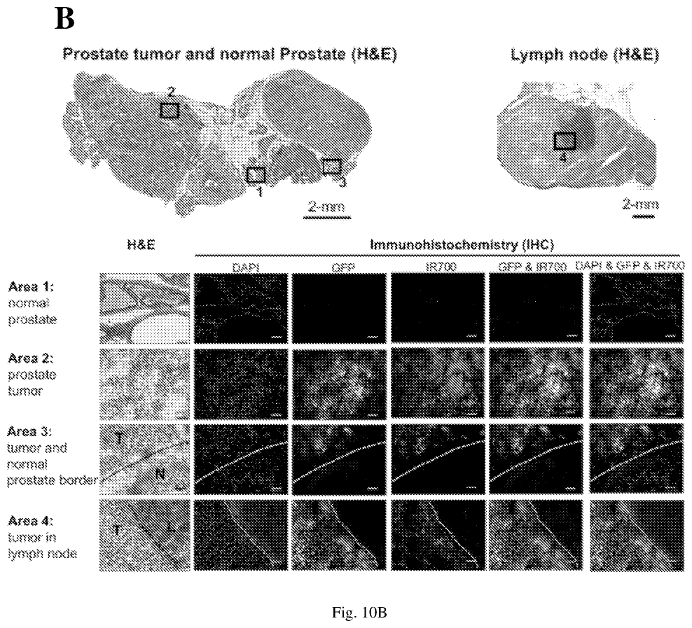

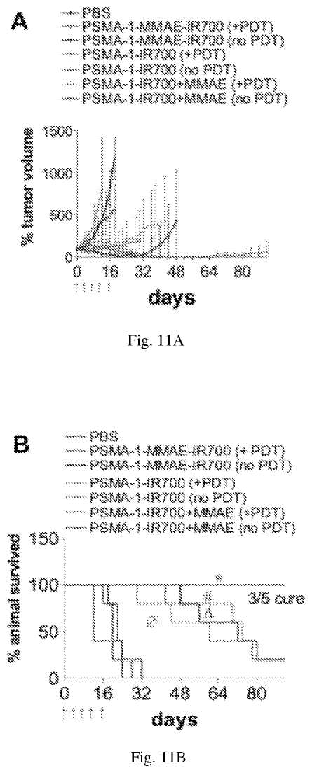

Competition binding experiments demonstrated that the complexity of the drug conjugates did not impact their binding affinity to PSMA; PSMA-1-VcMMAE-Cy5.5 showed an IC₅₀ of 3.65 nM and PSMA-1-McMMAE-Cy5.5 had an IC₅₀ of 4.88 nM, both similar to unconjugated PSMA-1, IC₅₀=2.30 nM and significantly lower than the parent ZJ24 ligand, IC₅₀=11.73 nM (FIG. 11C).

To investigate if PSMA-targeted drug conjugates were cleavable by cathepsin B, the two conjugates were incubated with or without the enzyme and chromatographed by HPLC. When the conjugates were incubated with PBS, both PSMA-1-VcMMAE-Cy5.5 and PSMA-1-McMMAE-Cy5.5 were stable (FIG. 1D). In the presence of cathepsin B, PSMA-1-VcMMAE-Cy5.5 degraded rapidly with a half-life at 0.33 h, releasing intact MMAE, while PSMA-1-McMMAE-Cy5.5 remained intact (FIG. 1D). The results demonstrated that PSMA-1-VcMMAE-Cy5.5 can be cleaved by cathepsin B, while PSMA-1-McMMAE-Cy5.5 is not cleavable by the protease.

To test if PSMA-targeted drug conjugates could selectively kill PSMA-expressing cells, cytotoxicity studies were performed in both PSMA-positive PC3pip and PSMA-negative PC3flu cells. PSMA-1-VcMMAE-Cy5.5 was about 20-fold more potent at killing PSMA-positive PC3pip cells (IC₅₀=0.84 nM) than PSMA-negative PC3flu cells (IC₅₀=17.0 nM) (FIG. 11E), while the free drug MMAE showed no selectivity between PSMA positive and negative cells. In contrast, PSMA-1-McMMAE-Cy5.5 was much less potent in cell killing, no IC₅₀ values could be obtained for either PC3pip and PC3flu cells for the concentrations tested, suggesting PSMA-1-VcMMAE-Cy5.5 acts as a prodrug dependent on protease activation. To confirm protease dependent activation of PSMA-1-VcMMAE-Cy5.5, we performed in vitro cell killing studies including 50 μM of the protease inhibitor E64 (FIG. 1E). Co-incubation with 50 μM of protease inhibitor E64 reduced the potency of PSMA-1-VcMMAE-Cy5.5. confirming that the conjugate worked as a prodrug.

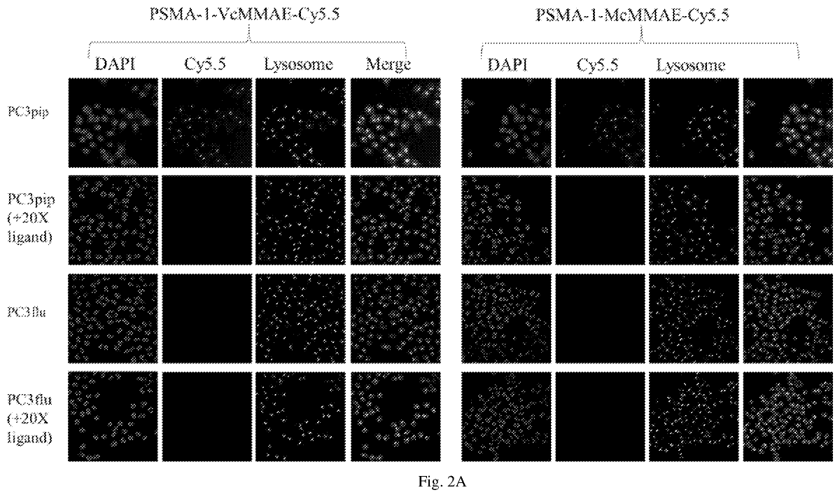

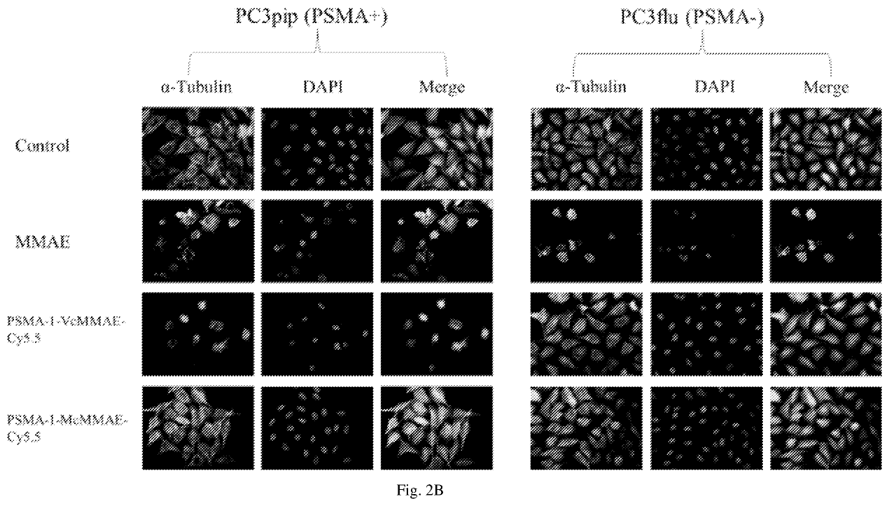

To determine if PSMA-targeted drug conjugates would result in selective cellular binding and uptake, in vitro uptake studies were performed. Similar levels of fluorescence uptake into PC3pip cells was observed after treatment with either PSMA-1-VcMMAE-Cy5.5 or PSMA-1-McMMAE-Cy5.5 and was localized to the lysosomal compartment of the cells (FIG. 2A). Competition studies indicated that binding of the two conjugates was selective and specific for the PSMA receptor expressed on the PC3pip cells.

It is known that MMAE inhibits cell division by destabilization of α-tubulin. To further validate the biological consequence of MMAE-conjugate treatment, in vitro immunofluorescence staining of α-tubulin was performed (FIG. 2B). Incubation with free MMAE led to non-selective tubulin disruption of the microtubule network in both PC3pip and PC3flu cells, causing them to round up and lose the spindle structure. PSMA-1-VcMMAE-Cy5.5 showed selective disruption of tubulin in PC3pip cells but not in PC3flu cells. In contrast, no obvious disruption of tubulin was observed in PC3pip or PC3flu cells when treated with the same concentration of the non-cleavable PSMA-1-McMMAE-Cy5.5.

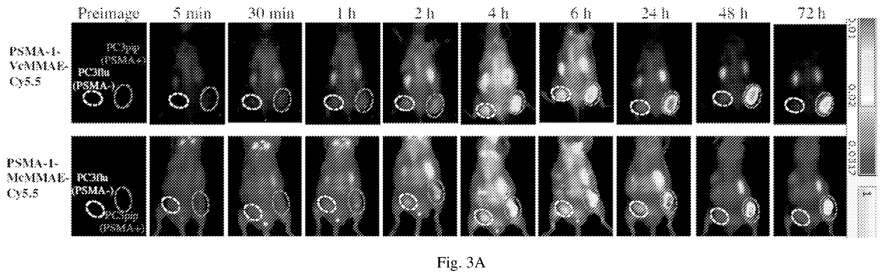

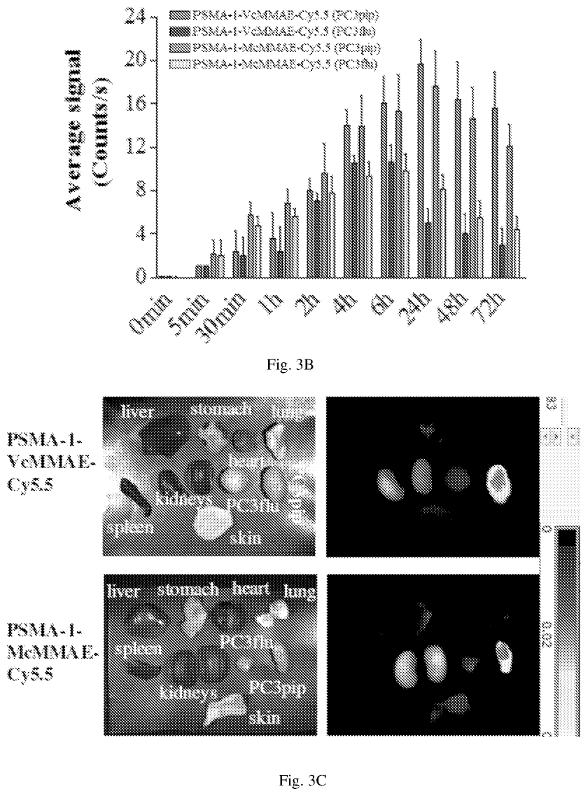

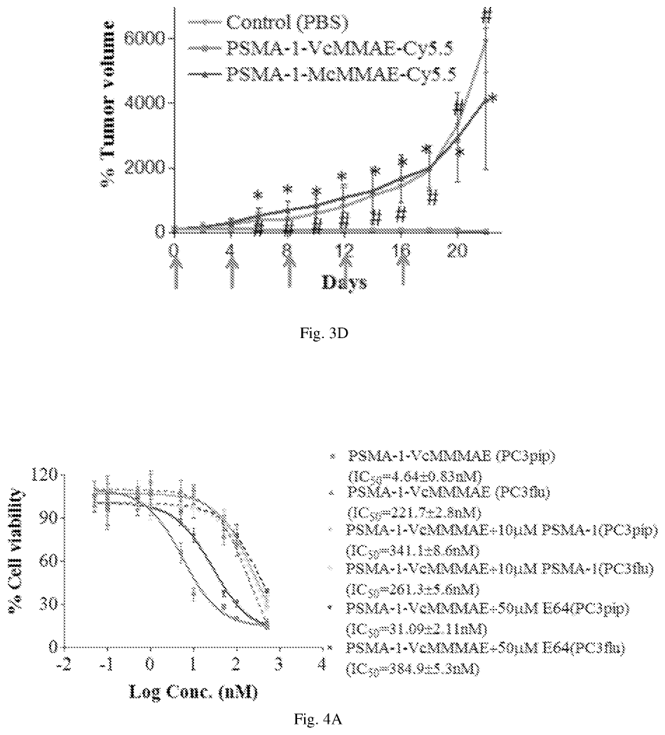

To demonstrate selective tumor uptake in vivo, mice bearing both PC3flu and PC3pip tumors were injected with 40 nmol/kg of PSMA-1-VcMMAE-Cy5.5 or PSMA-1-McMMAE-Cy5.5 and uptake monitored via fluorescence imaging over time (FIG. 3). Similar selective uptake was observed in PC3pip tumors for both conjugates, peaking at 24-hour post-injection followed by a prolonged clearance. At 24-h post-injection there was little to no detectable uptake in PSMA-negative PC3flu tumors (FIGS. 3A-B). Ex vivo imaging of tissues at 72-hour post-injection showed that fluorescence was mainly retained in the PSMA-expressing tumor; no/minimal fluorescence was detected in other organs (FIG. 3C).

We next compared the antitumor activity of the two conjugates in mice bearing a flank PC3pip tumor. Each animal received 160 nmol/kg of PSMA-1-VcMMAE-Cy5.5 or PSMA-1-McMMAE-Cy5.5 through tail vein injection every four days with a total of 5 doses. (Dosing was based on previously published dose and schedules for antibody-drug conjugates). Mice were then imaged and tumors were measured by caliper. Untreated PC3pip tumor grew rapidly achieving a 50-fold increase in size by day 22 (FIG. 3D). Quantitative Maestro imaging showed that the average fluorescence signal in PC3pip tumors was similar for each theranostic injected and peaked in PC3pip tumors 24-hour after each injection (day 1, day 5, day 9, day 13 and day 17). Mice treated with the non-cleavable conjugate, PSMA-1-McMMAE-Cy5.5, showed similar growth rate as that measured for untreated mice. In contrast, administration of PSMA-1-VcMMAE-Cy5.5 effectively inhibited tumor growth as early as day 6 after the initial dose, indicating that prodrug strategy with cleavable linker is imperative for the antitumor activity of the conjugates. The changes in body weight were similar between mice receiving PBS or targeted drug conjugates, suggesting that the drug treatment is not very toxic to the animals.

### Efficacy of PSMA-Targeted Prodrug Conjugates In Vitro

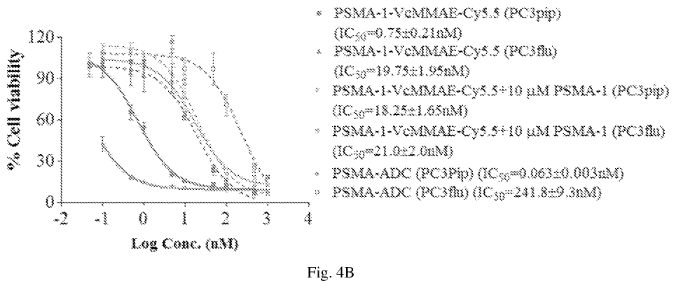

Having confirmed that prodrug strategy is crucial for the antitumor activity, we performed studies to further explore the antitumor activity of PSMA-1-VcMMAE and PSMA-1-VcMMAE-Cy5.5 (FIG. 1A) in vitro and in vivo (FIG. 4). The incorporation of Cy5.5 will not only provide information of tumor response to the treatment, but, we have demonstrated, will also modulate blood retention. Both PSMA-1-VcMMAE (FIG. 4A) and PSMA-1-VcMMAE-Cy5.5 (FIG. 4B) selectively killed PC3pip cells with IC₅₀'s of 4.64 and 0.75 nM, respectively. Removal of the Cy5.5 moiety, however, increased the IC₅₀ of PSMA-1-VcMMAE by 6.2-fold against PC3pip cells and 11.1-fold against PC3flu cells, suggesting a reduction in absolute efficacy. For PSMA-1-VcMMAE the ratio of IC₅₀'s between PC3pip and PC3flu cells increased to 48.2, compared to a ratio of 26.3 for PSMA-1-VcMMAE-Cy5.5, suggesting that removal of Cy5.5 increased the selectivity of the probe for PSMA-expressing cells. In the presence of 10 IM of PSMA-1, the potency to kill PC3pip cells for both agents was reduced to the same potency to kill PC3flu cells, again indicating that the killing is dependent on PSMA binding. PSMA-1-VcMMAE activity was also susceptible to E64 protease inhibition suggesting that it also worked as a prodrug, FIG. 4A. We next compared the cytotoxicity of these agents with antibody-conjugated MMAE (PSMA-ADC), which entered clinical trials but failed due to toxicity. It was found that PSMA-ADC was the most potent (IC₅₀=0.063 nM), followed by PSMA-1-VcMMAE-Cy5.5 (IC₅₀=0.75 nM), and PSMA-1-VcMMAE (IC₅₀=4.64 nM). PSMA-ADC had better selectivity defined by differential cytotoxicity ratio between PC3pip and PC3flu cells of 3838-fold compared to PSMA-1-VcMMAE (48-fold) and PSMA-1-VcMMAE-Cy5.5 (26-fold). PSMA-ADC (IC₅₀=241.8 nM) and PSMA-1-VcMMAE (IC₅₀=221.7 nM) had similar potency to kill non-PSMA expressing PC3flu cells, suggesting the antibody's greater affinity resulted in efficacy differences against PC3pip cells.

### In Vivo Studies of Toxicity and Efficacy

The maximum tolerated dose (MTD) was determined in tumor-free athymic nude mice to determine the toxicity of the conjugates. Loss of 20% body weight or any overt signs of toxicity was used as an end point. We compared the MTD of free drug MMAE, PSMA-targeted drug conjugates and PSMA-ADC. MTDs of single dose i.v. injection of MMAE, PSMA-1-VcMMAE, PSMA-1-VcMMAE-Cy5.5 and PSMA-ADC were 700 nmol/kg, 7640 nmol/kg, 480 nmol/kg and 640 nmol/kg, respectively, highlighting the superior safety (10-fold or greater) of PSMA-1-VcMMAE compared to all the other drug derivatives.

For in vivo antitumor activity, we first studied in vivo potency of PSMA-1-VcMMAE in nude mice bearing heterotopic PC3pip tumors. Mice received drugs intravenously every 4 days with a total of 5 doses. In PBS control groups the tumor grew rapidly resulting in animal death within 30 days. In contrast treatment with PSMA-1-VcMMAE showed the ability to inhibit tumor growth and prolong animal survival in a dose dependent manner. At the highest dose tested (3820 nmol/kg, ½ of its MDT), all 5 mice survived the 90-day experimental time and 3 out of 5 mice were tumor free resulting in 60% cure. No body weight loss was observed even at the highest dose tested. It was also found that PSMA-1-VcMMAE was significantly more effective at inhibiting PSMA-positive PC3pip tumor growth than PSMA-negative PC3flu tumor growth when used at a lower dose of 955 nmol/kg (P=0.0493). We then administered PSMA-1-VcMMAE in mice bearing orthotopic PC3pip tumors, which mimic human prostate cancer in a more realistic way. Inhibition of tumor growth and extension of animal survival time were observed in the orthotopic PC3pip tumor models, and significant differences were observed at the dose of 1910 (P=0.0449) and 3860 nmol/kg (P=0.0019) when compared to the PBS control, with 1 mouse that was tumor free at the dose of 3860 nmol/kg. Initial changes in body weight were similar among the tested groups.

We have reported that Cy5.5 can change the pharmacokinetics of the conjugate drastically. We therefore tested antitumor activity of PSMA-1-VcMMAE-Cy5.5 and also included equimolar doses of free MMAE and PSMA-ADC as controls in the study. Free MMAE was relatively ineffective at inhibiting tumor growth, even at the dose of 160 nmol/kg, all mice died within 36 days. In contrast, PSMA-1-VcMMAE-Cy5.5 effectively inhibited tumor growth, and expanded animal survival times. Significantly at 160 nmol/kg of PSMA-1-VcMMAE-Cy5.5 the tumors were completely eliminated in every case for a period of time and resulted in a cure rate (no tumors at end of study) of 60% for the 90-day experimental period. Changes in body weight were similar in each regimen and control group in the first 16 days, however, increased body weight was observed in PSMA-1-VcMMAE-Cy5.5 treated groups due to reduced disease burden. Compared to PSMA-1-VcMMAE-Cy5.5, all animals treated with 160 nmol/kg of PSMA-ADC survived the 90-day experimental time with 40% cure, the difference between the two groups was not significant (P=1.000).

We then tried PSMA-1-VcMMAE-Cy5.5 in orthotopic PC3pip tumor. No significant body weight loss was observed in mice treated with PSMA-1-VcMMAE-Cy5.5 while the PBS control mice lost body weight due to increased tumor burden. Tumor volume in PSMA-1-VcMMAE-Cy5.5 treated animals was significantly reduced and animal survival were dramatically extended (P=0.0026) with 40% of cure at day 90.

The mortality of prostate cancer is mainly due to metastasis and metastatic castration resistant prostate cancer is the most deadly and difficult form of the disease to treat. To test the effectiveness of PSMA-1-MMAE-Cy5.5 against metastatic disease, we developed a metastatic prostate cancer model using intracardiac injection of GFP-expressing PC3pip cells. We then used this model to assess the effectiveness of our drug conjugate. Treatment was initiated 1 week after cardiac injection of tumors cells, and mice received 160 nmol/kg of PSMA-1-VcMMAE-Cy5.5 every 4 days with a total of 5 doses. After treatment, no significant body weight loss was observed. The control mice died with 28 days. Maestro images of control mice showed GFP signal in tibia, spine, liver, spleen, kidney and testis, indicating tumors had metastasized to these sites. In contrast, all 5 treated mice survived the 90-day experimental period, and no GFP signal was observed in throughout the mice body, indicating a 100% response. PSMA-1-VcMMAE was also found to have the ability to prolong animal survival time with no body weight loss in the metastatic PC3pip model.

To show versatility of the antitumor activity of PSMA-1-VcMMAE-Cy5.5, we tested it in mice bearing heterotopic C4-2 tumors, which are androgen-independent prostate cancer cells endogenously expressing PSMA. PSMA-1-VcMMAE-Cy5.5 showed the ability to successfully inhibit C4-2 tumor growth with no weight loss and prolonged animal survival significantly (P=0.0018) with a 40% cure rate.

The targeted delivery of potent cytotoxic agents has emerged as a promising strategy for the treatment of cancer. In this study, we demonstrate that targeted delivery of MMAE can be more efficacious and less toxic than MMAE. More remarkably, PSMA-1-MMAE has an improved therapeutic index compared to PSMA-ADC.

### Prodrug Strategy is Vital for Antitumor Activity

Targeted strategies require three components: the targeting ligand which can be an antibody, small molecular ligand or aptamer; the cytotoxic payload; and the linker. PSMA is a well-established biomarker for prostate cancer. We used our PSMA-1 ligand to target and deliver the potent microtubule polymerization inhibitor, MMAE, to PSMA-expressing prostate cancer cells. For linker selection, there are two types of linker-cleavable and non-cleavable linkers. To determine which linker would be most suitable, we tried both the cathepsin cleavable maleimido-caproyl-Val-Cit-PABC linker (Vc) (PSMA-1-VcMMAE-Cy5.5) (FIG. 1A) and the noncleavable maleimido-caproyl linker (Mc) (PSMA-1-McMMAE-Cy5.5) (FIG. 1B). It was found that although both conjugates selectively accumulated in PC3pip cells in vitro (FIG. 2) and in vivo (FIG. 3), effective antitumor activity was only observed with conjugates using the self-immolative cleavable “Vc”-linker, which concurs with the report that MMAE is most effective in its natural form and therefore not suitable for derivatization with non-cleavable linkers. Once entering the cells, the conjugates located mainly in the lysosomes (FIG. 2A), where cathepins are highly expressed and active. Inclusion of protease inhibitors diminished drug efficacy indicating that lysosomal accumulation and protease activation of PSMA-1-VcMMAE-Cy5.5 liberated free MMAE resulting in disruption of α-tubulin and cancer cell death (FIG. 1E).

### Targeting Improves the Therapeutic Profile of MMAE

MMAE is a favored drug payload for ADCs, with many in clinical trials, and is usually conjugated to the antibody through a cathepsin B cleavable Vc linker, exploiting the ability to release drug intracellularly. Here, we replaced the antibody with a PSMA targeting ligand (PSMA-1) and developed a small-molecule-drug conjugate to reduce the cost and shorten the circulation time, potentially reducing the off-target toxicities resulting from longer blood half-lives. In vitro comparison of the cytotoxicity of free MMAE (FIG. 1E) with PSMA-ADC and our PSMA-1-drug conjugates (FIG. 4) showed that free MMAE killed both PC3pip and PC3flu cells with no selectivity. In contrast, PSMA-ADC and PSMA-1-drug conjugates selectively killed PC3pip cells with PSMA-ADC being the most potent and selective. The improved potency and selectivity of PSMA-ADC maybe due to its significantly higher affinity for PSMA (picomolar) than the PSMA-1 ligand used (nanomolar). PSMA-1-VcMMAE-Cy5.5 was found to be more potent than PSMA-1-VcMMAE in vitro, but less selective. The Cy5.5 renders greater hydrophobicity to PSMA-1-VcMMAE-Cy5.5. It has been reported that higher hydrophobicity can lead to increased cellular uptake and therefore higher cytotoxicity, which may explain the higher cytotoxicity of PSMA-1-VcMMAE-Cy5.5. Further increased hydrophobicity may have increased non-selective drug uptake into PC3flu cells, contributing to the decreased selectivity of PSMA-1-VcMMAE-Cy5.5.

Comparing the maximum tolerated dose (MTD) of MMAE, PSMA-ADC and PSMA-1-drug conjugates, PSMA-1-VcMMAE was more than 10-fold better than MMAE. This is likely due to targeted drug delivery. Interestingly, the MTD for PSMA-ADC was much lower than our lower affinity small molecule conjugate PSMA-1-VcMMAE, but very similar to PSMA-1-VcMMAE-Cy5.5. Although the MTDs of PSMA-ADC and PSMA-1-VcMMAE-Cy5.5 were lower than MMAE itself, their in vivo effective doses were disproportionately better. At the dose of 160 nmol/kg, PSMA-1-VcMMAE-Cy5.5 and PSMA-ADC potently inhibited PC3pip tumor growth leading to significant prolonged survival time, while MMAE did not show any effectiveness at the same dose. No significant difference in animal survival time was observed between PSMA-1-VcMMAE-Cy5.5 and PSMA-ADC treatment groups (P=1.000), suggesting that PSMA-1-VcMMAE-Cy5.5 has similar efficacy to PSMA-ADC. Compared to PSMA-ADC and PSMA-1-VcMMAE-Cy5.5, PSMA-1-VcMMAE needed a higher dose to achieve the same treatment effect when given at the same dosing schedule, which is likely attributed to differences in pharmacokinetic characteristics. Our previous studies have shown that the hydrophobicity/hydrophilicity of conjugated dyes can dramatically impact the pharmacokinetics of the conjugates, with more hydrophilic conjugates resulting in a shorter time-to-peak accumulation in tumors and shorter circulation times. It may be that integration of hydrophobic Cy5.5 in PSMA-1-VcMMAE-Cy5.5 provided elongated circulation of the drug conjugate in vivo, maximizing antitumor activity. The difference in pharmacokinetics may contribute to the different MTDs of PSMA-1-VcMMAE, PSMA-1-VcMMAE-Cy5.5 and PSMA-ADC. Future pharmacokinetic studies are required to confirm our hypothesis and optimize these agents. It was noticed that animals treated by the drug conjugates showed no body weight loss due to the treatment, indicating low toxicity of the drug conjugates. Biodistribution studies using PSMA-1-VcMMAE-Cy5.5 showed that the fluorescence from the conjugate was associated with PC3pip tumors with minimal signals observed in the liver, spleen, lungs etc. This may indirectly indicate the low toxicity of the conjugate in other organs. However, MMAE will be detached from Cy5.5 in vivo, the information obtained from fluorescence imaging may not be directly correlated to the biodistribution of the drug. Detailed biodistribution and toxicity studies are still needed.

Notably, PSMA-1-VcMMAE and PSMA-1-VcMMAE-Cy5.5 showed the ability to significantly prolong the survival time of animals in metastatic PC3pip tumor model. In spite of the fact that the treatment initiated one week after tumor inoculation, before the metastasis was detectable using imaging, it is encouraging that it exhibited the ability to successfully prevent tumor metastasis. As a significant percentage of patients with prostate cancer die from the metastatic form of the disease due to lack of effective treatment options, it is critical to develop potent treatments that can effectively eradicate cancer cells and control metastatic tumors. We also found that both PSMA-1-VcMMAE and PSMA-1-VcMMAE-Cy5.5 greatly inhibited tumor growth when given every 7 days.

PSMA-1-VcMMAE utilizes the same targeting strategy as PSMA-ADC, but has shorter circulation time due to its smaller size and can be easily excreted from the body leading to reduced toxicity. PSMA-1-VcMMAE effectively inhibited tumor growth starting at the dose of 191 nmol/kg, and its' MTD was at 7640 nmol/kg, resulting in a therapeutic index of 40. The TI of PSMA-1-VcMMAE-Cy5.5 is 480/80=6. PSMA-ADC was reported to be effective starting at 2.0-3.0 mg/kg (53 nmol/kg-80 nmol/kg), its MTD was at 640 nmol/kg resulting in its TI at 8-12. By replacing the antibody with our PSMA-1 ligand, PSMA-1-VcMMAE dramatically improved the therapeutic index. These characteristics will help it avoid the problems of PSMA-ADC found in clinical trials. A theranostic such as PSMA-1-VcMMAE-Cy5.5 will allow modification of pharmacokinetics and will play a key role in initial drug development by enabling visualization of drug-target engagement, tumor size, and providing feedback information on the therapy status. Even though after prodrug activation by proteases Cy5.5 is released and physically separated from MMAE, this theranostic molecule provides significant data about drug targeting and allows greater ease and less time for small animal optimization studies. It is the first example of combining targeted small molecules with therapeutics and fluorophores for a theranostic approach to develop targeted drugs for prostate cancer.

In summary, we have developed small-molecule-based prodrugs for the treatment of prostate cancer. Their antitumor activities were demonstrated not only in androgen independent PC3pip heterotopic, orthotopic and metastatic models but also in androgen independent C4-2 tumor models that endogenously express PSMA, with no toxicity observed.

### Example 2

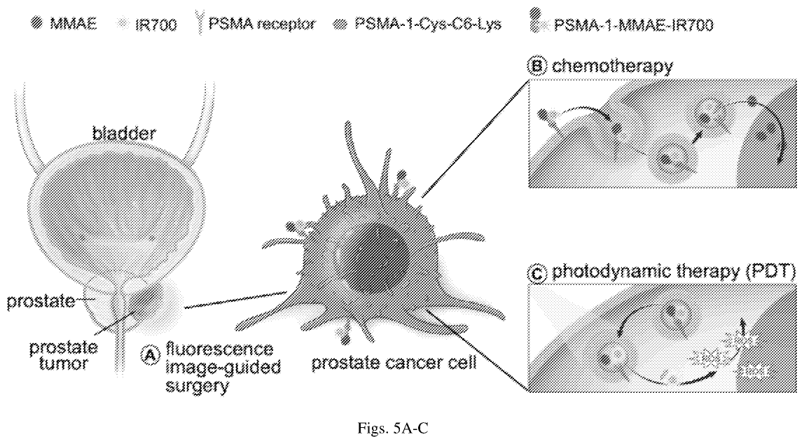

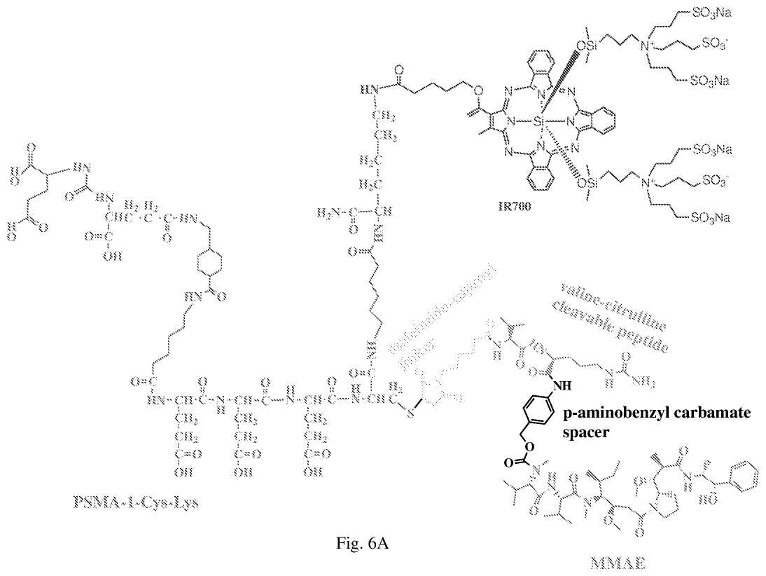

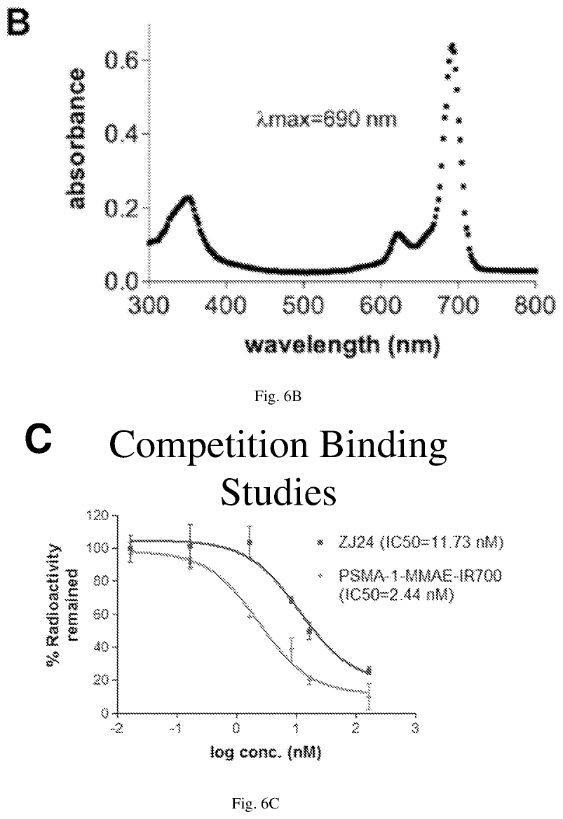

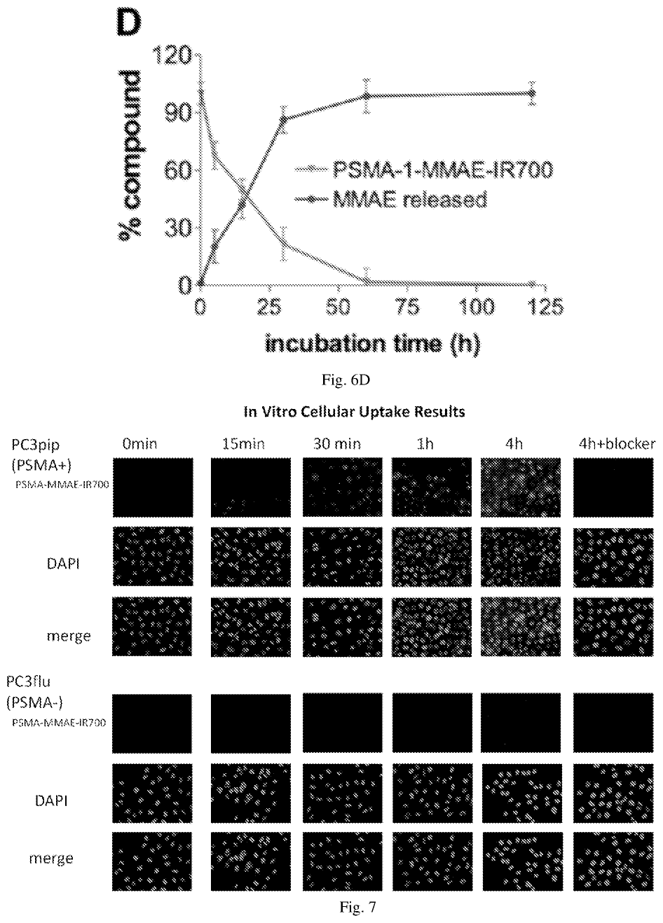

In this Example, we developed a multifunctional theranostic approach that combines a cytotoxic drug (MMAE), a photosensitizer (IR700) and a low molecular weight PSMA targeting ligand (PSMA-1-Cys-C6-Lys) into a single molecule, PSMA-1-MMAE-IR700, that selectively and simultaneously delivers both chemotherapeutic drugs and photosensitizers to cancer cells (FIGS. 5 and 6A). In addition, IR700 emits near infrared light at around 700 nm with a fluorescence quantum yield at 0.24, therefore, this new approach can also be used for near infrared fluorescence detection of cancers (FIG. 5). Our results showed that PSMA-1-MMAE-IR700 can be selectively delivered to PSMA-expressing cancer cells. More importantly, the PSMA-targeted covalent combination of PDT and chemotherapy agents showed significantly improved antitumor activity compared to individually PSMA-targeted treatment with PDT or chemotherapy, or the non-covalent simultaneous addition of both PSMA-targeted PDT and free MMAE.

### Methods

### Materials

PSMA-1-Cys-C6-Lys (Glu-CO-Glu′-Amc-Ahx-Glu-Glu-Glu-Cys-C6-Lys) was synthesized manually by solid phase peptide synthesis method as reported previously. VcMMAE was purchased from Creative Biolabs (Shirley, NY). IRDye700 NHS ester was purchased from Li-Cor Biosciences (Lincoln, NE). PSMA-1-IR700 was synthesized as previously reported. (S)-2-(3-((S)-5-amino-1-carboxypentyl)-ureido)pentanedioic acid (ZJ24) was custom made by Bachem Bioscience Inc (Torrance, CA). Tritium labeled ZJ24 (N—[N—[(S)-1,3-dicarboxypropyl]-carbamoyl]-S-[3H]-methyl-L-cysteine, 3H-ZJ24) was custom synthesized by GE Healthcare Life Sciences (Chicago, IL). All the other chemicals were purchased from Sigma-Aldrich (St. Louis, MO).

### High Performance Liquid Chromatography (HPLC)

HPLC was performed on a Shimadzu HPLC system equipped with an SPD-20A prominence UV/visible detector and monitored at 220 nm and 254 nm [57, 58, 60]. Semi-preparative HPLC was achieved using Luna 5m C18(2) 100 Å column (250 mm×10 mm×5 mm; Phenomenex) at a flow rate of 2.5 mL/min. Analytical HPLC was performed using an analytical Luna 5m C18(2) 100 Å column (250 mm×4.6 mm×5 mm; Phenomenex) at a flow rate of 0.8 m/min. The gradient used to purify PSMA-1-MMAE-IR700 was 10% to 90% acetonitrile against 25 mM triethyl-ammonium acetate (TEAA, pH 7.5) over 30 min.

### Synthesis of PSMA-1-MMAE-IR700

PSMA-1-VcMMAE was synthesized as previously reported. Briefly, PSMA-1-Cys-C6-Lys (2.6 mg, 2 μmol) was dissolved in phosphate buffered saline (PBS), the pH of the solution was adjusted to 7.5-8.0, then Vc-MMAE (3.0 mg, 2.2 μmol) (BOC Sci.) in 500 μL of DMF was added. The reaction mixture was allowed to react at room temperature for 1 h. PSMA-1-VcMMAE was then purified by semi-preparative HPLC and lyophilized. Retention time: 17.6 min. Mass spectrum (MS) (C₁₂₃H₁₉₅N₂₃O₃₇S). calculated: 2618.3. found: 2619 (M+1). Purified PSMA-1-VcMMAE (1.3 mg, 0.5 μmol) was dissolved in 0.5 mL PBS. The pH of the solution was adjusted to 7.5, then IRDye700 NHS ester (1.9 mg, 1 mol, Li-Cor Inc.) in 0.5 mL PBS was added. The mixture was allowed to react at room temperature overnight. PSMA-1-MMAE-IR700 was then purified by HPLC. Yield: 1.6 mg, 76%. Retention time: 19.9 min. Mass spectrum (MS) (ammonium salt: C₁₉₃H₃₀₂N₃₈O₆₁S₇Si₃). calculated: 4439. found: 971 ([M−4 NH₄-C₁₄H₃₀NO₁₀S₃Si]/4), 1091 ([M−4 NH]/4), 1295 ([M−4 NH-₄ C₁₄H₃₀NO₁₀S₃Si]/3), 1456 ([M−4 NH₄]/3), 1943 ([M−4 NH₄-C₁₄H₃₀NO₁₀S₃Si]/2).

### Cell Culture

PSMA-positive PC3pip and PSMA-negative PC3flu cells were maintained in RPMI medium with 10% Fetal Bovine Serum at 37° C. and 5% CO₂ under a humidified atmosphere. The cells were last sorted and checked by western blot in 2021.

### Competition Binding Studies

PC3pip cells (5×105) was suspended in 200 μL of 50 mM Tris buffer, pH 7.5. The cells were incubated at 37° C. with different concentrations of PSMA-1-MMAE-IR700 or ZJ24 in the presence of 12 nM of 3H-ZJ24 for 1 h. The cells were then washed 3 times with cold PBS and cell-associated radioactivity was measured by scintillation counting. The concentration required to inhibit 50% of the binding (IC₅₀) was determined by GraphPad Prism 3.0. Studies were performed in triplicate.

### Cathespin Cleavage Studies

Enzymatic cleavage study of PSMA-1-MMAE-IR700 was performed as previously described. PSMA-1-MMAE-IR700 was incubated with activated human liver cathespin (Anthens Research and Technology, Anthens, GA) at 37° C. At different incubation time, 40 μL of the solution was placed into tubes loaded with 1 μL of 1 mM E64 protease inhibitor. The mixture was vortexed and then stored at −80° C. for future HPLC analysis. Studies were performed in triplicate. In vitro cellular uptake studies cells were plated on coverslips at about 60-70% confluency. Twenty-four hours later, cells were incubated with 50 nM of PSMA-1-MMAE-IR700. After incubation for various times (15 min, 30 min, 1 h and 4 h), cells were washed twice with cold RPMI 1640, fixed with 4% paraformaldehyde and counterstained with 4′,6-diamidino-2-phenylindole (DAPI). Images were taken using a Leica DM4000B fluorescence microscope (Leica Biosystems, Buffalo Gove, IL) at 40×. Blocking experiments were performed by co-incubation of cells with 50 nM of PSMA-1-MMAE-IR700 and 100× of PSMA-1 ligand. Studies were performed in triplicate.

### In Vitro Cytotoxicity Studies

PC3pip and PC3flu cells were plated at 3,000 cells/well in 96-well plates. Twenty-four hours later, drugs (PSMA-1-MMAE-IR700 or PSMA-1-IR700) of different concentrations were added. After incubation at 37° C. in the dark for 24 h, cell viability was determined by CellTiter 96© aqueous one solution cell proliferation assay using absorbance at 490 nm (Promega Biotech, Madison, MI). The concentration required to inhibit 50% of cell growth (IC₅₀) was determined by GraphPad Prism 3.0. To test the cytotoxicity of PSMA-1-MMAE-TR700 with light irradiation, drugs at a final concentration of 5 nM were added to cells and incubated in the dark for 24 h. Cells were then washed 3 times with RMPI 1640 and then irradiated with 690 nm light (L690-66-60, Marubeni America Co, New York, NY). Cells were incubated in the dark for another 24 h. Cell viability was then determined by CellTiter 96© aqueous one solution cell proliferation assay (Promega Biotech, Madison, MI). The coefficient drug interaction (CDI) was calculated as follows: CDI=AB/(A×B), where AB is the ratio of the absorbance of combination treatment groups (PSMA-1-MMAE-IR700 with light irradiation) to the absorbance of control groups; A or B is the absorbance of single treatment group (PSMA-1-MMAE-IR700 without light or PSMA-1-IR700 with light irradiation) to the absorbance of control groups. CDI <1, =1 and >1 indicates synergistic, additive or antagonistic effect.

### In Vivo Fluorescence Imaging Studies

Animal experiments were approved by the animal care and use committee at Case Western Reserve University (IACUC #150033). Six to eight-week-old male Balb/c athymic nude mice (Jackson Laboratory, Bar Harbor, ME) were implanted subcutaneously with 1×10⁶ of PC3pip (right flank) and PC3flu (left flank) cells in 100 μl of matrigel. Animals were ready to use when tumor diameter reached 10 mm, about two weeks. Animals received 100 nmol/kg of PSMA-1-MMAE-IR700 through tail vein injection and were imaged at different time points by Maestro In vivo Image System (Perkin Elmer, Waltham, MI) using the yellow filter set (excitation 575-605 nm, emission 645 nm longpass). During imaging, mice were anesthetized by isoflurane. At 48 h post injection, mice were sacrificed and tissues such as heart, lung, liver, kidneys, stomach, tumors were harvested for ex vivo imaging. Multispectral images were unmixed into their component spectra and average fluorescence signals were quantified by creating regions of interest. Experiments were performed in 5 mice.

Orthotopic PC3pipGFP prostate cancer models were established as previously described using green fluorescence protein (GFP) transfected PC3pip cells. Mice bearing orthotopic PC3pipGFP tumors were injected with 100 nmol/kg of PSMA-1-MMAE-IR700. Mice were imaged at 1 h post injection by Maestro using the yellow filter set for PSMA-1-MMAE-IR700 and blue filter set for GFP (excitation 445-490 nm, emission 515 nm longpass). Mice were then euthanized and primary tumor was removed to expose lymph nodes. The mice were imaged again. Resected primary tumor and lymph nodes were snap-frozen in optimal cutting temperature (OCT) compound and sectioned. The slides were subjected to hematoxylin and eosin (H&E) staining, and adjacent set of slides were counter stained with DAPI and observed under Leica DM4000B fluorescence microscope at 10× to visualize DAPI, GFP and PSMA-1-MMAE-IR700. Experiments were repeated in three mice.

### In Vivo Antitumor Efficacy Studies

The effect of PSMA-1-MMAE-IR700 were tested in mice bearing PC3pip tumors. Animals with tumor size at about 100 mm3 were used for the study (tumor volume=Length×width2/2). Animals were divided into 7 groups: (1) mice receiving PBS; (2) mice receiving 100 nmol/kg PSMA-1-MMAE-IR700 with PDT; (3) mice receiving equal doses of PSMA-1-MMAE-IR700 to group 2, but not receiving PDT; (4) Mice receiving equal doses of PSMA-1-IR700 to group 2 with PDT treatment; (5) Mice receiving equal doses of PSMA-1-IR700 to group 2 without PDT; (6) mice receiving equal doses of PSMA-1-IR700 to group 2+free MMAE with PDT (MMAE normalized to that delivered by PSMA-1-MMAE-IR700); and (7) mice receiving equal doses of PSMA-1-IR700 to group 2+free MMAE without PDT. Each group had 5 mice. Animals received drugs through tail vein injection on day 0, 4, 8, 12 and 16 and treated with 50 J/cm2 of 690 nm light at 1 h post-injection on these injection days. The dose and schedule was based on previous PSMA-1-VcMMAE work and were not optimized. Animals were imaged before and after PDT. Mice were monitored every other day for 90 days. Animals were euthanized when tumors became too large (diameter >20 mm) or animals were moribund. Data were reported as body weight over time, tumor size over time and Kaplan-Meier survival plots.

### Immunofluorescent Detection of Apoptosis

Animals bearing PC3pip tumors were divided into 7 groups: (1) mice receiving PBS; (2) mice receiving 100 nmol/kg PSMA-1-MMAE-IR700 with PDT; (3) mice receiving equal doses of PSMA-1-MMAE-IR700 to group 2, but not receiving PDT; (4) Mice receiving equal doses of PSMA-1-IR700 to group 2 with PDT treatment; (5) Mice receiving equal doses of PSMA-1-IR700 to group 2 without PDT; (6) mice receiving equal dose of PSMA-1-IR700 to group 2+free MMAE with PDT (MMAE normalized to that delivered by PSMA-1-MMAE-IR700); and (7) mice receiving equal dose of PSMA-1-IR700 to group 2+free MMAE without PDT. Animals were treated with one single dose and were sacrificed at 4-day post treatment. Tumors were snap-frozen in OCT, cut into 10 μm thick sections and fixed on slides. Induction of apoptosis by the treatment was determined by rabbit polyclonal anti-Caspase-3 antibody (Abcam, Cambridge, UK). A goat anti-rabbit polyclonal antibody labeled by Alexa Fluor-594 was used as secondary antibody (Abcam, Cambridge, UK). The presence of apoptosis was determined by fluorescence images under Leica DM4000B fluorescence microscope at 10×. H&E staining of tumor tissues was performed in adjacent sections to check the histology of the tumors. Experiments were repeated in 5 mice.

### Statistics

Student t-test was used to compare inter-group differences. Kaplan-Meier survival data were analyzed by SAS 9.4 using log-rank tests. A p value <0.05 was considered statistically significant for all comparisons.

### Results

### Chemistry and In Vitro Competition Binding Studies

To synthesize PSMA-1-MMAE-IR700 (FIG. 6A), we used a self-immolative cathespsin cleavable Vc linker to conjugate MMAE to PSMA-1-Cys-C6-Lys. IR700 was conjugated to the PSMA-1 ligand through reaction of the Lys amine (—NH2) with NHS ester as no IR700 release is required for light activation. PSMA-1-MMAE-IR700 had a maximum absorbance (λmax) at 690 nm (FIG. 6B), which concurred with PSMA-1-IR700. In a competition binding assay (FIG. 6C), PSMA-1-MMAE-IR700 showed IC₅₀ at 2.44±0.43 nM, which was 4.4-fold greater than the related ligand ZJ24 (IC₅₀=10.71±0.89 nM) and similar to the PSMA-1 ligand. In the presence of cathepsin, PSMA-1-MMAE-IR700 degraded rapidly and release of MMAE was observed, supporting the prodrug strategy of PSMA-1-MMAE-IR700 (FIG. 6D). Stability studies found that PSMA-1-MMAE-IR700 was stable in PBS when incubated at 37° C. In mouse plasma, degradation of PSMA-1-MMAE-IR700 was observed with half-life at 17.2±2.8 h. Degradation of PSMA-1-MMAE-IR700 led to release of free MMAE.

### In Vitro Uptake Studies

To determine the selectivity of PSMA-1-MMAE-IR700, uptake studies were performed using both PSMA-positive PC3pip and PSMA-negative PC3flu cells. Fluorescence signal in PC3pip cells was observed as early as 15 min after incubation with PSMA-1-MMAE-IR700 and the signal intensity increased with extended incubation time (FIG. 7). In contrast, no fluorescence signal was observed in PC3flu cells. When PC3pip cells were co-incubated with PSMA-1-MMAE-IR700 and 100× of PSMA-1 ligand, the fluorescence signal was completely blocked, indicating that the binding of PSMA-1-MMAE-IR700 was specific to PSMA. Further confocal images showed that the fluorescence from PSMA-1-MMAE-IR700 co-localized to the lysosome compartment of the cells where cathepsins are highly expressed. The results concurred with our previous study with PSMA-1-VcMMAE-Cy5.5, suggesting the internalization of PSMA-1-MMAE-IR700 and its accumulation in lysosomes.

### In Vitro Cytotoxicity Studies

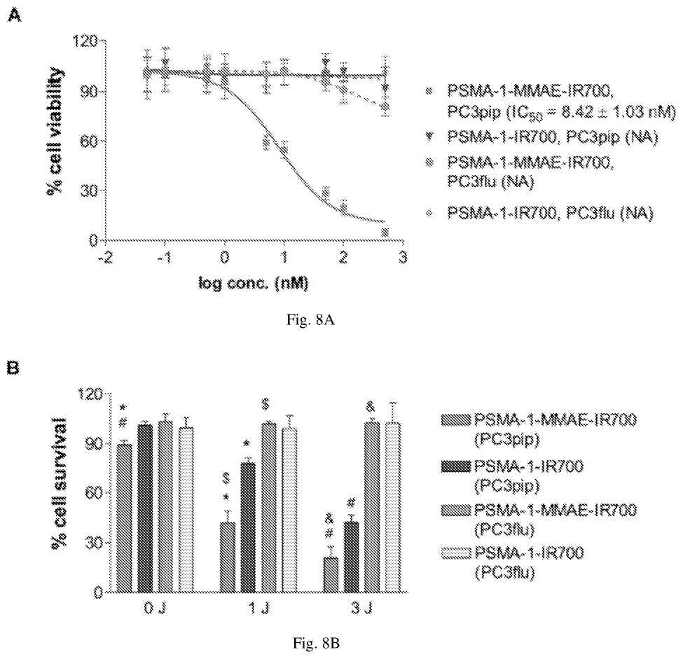

Cytotoxicity of PSMA-1-MMAE-IR700 was performed in both PSMA-positive PC3pip and PSMA-negative PC3flu cells to test if PSMA-1-MMAE-IR700 would selectively kill PSMA-positive cells. We first tested the cytotoxicity of PSMA-1-MMAE-IR700 and PSMA-1-IR700 in PC3pip and PC3flu cells without light treatment. After 24 h of incubation in the dark, PSMA-1-IR700 did not show any activity indicating that PSMA-1-IR700 is not toxic without light activation (FIG. 8A). PSMA-1-MMAE-IR700 effectively killed PC3pip cells with an IC50=8.42±1.03 nM, while it was much less potent in killing PC3flu cells and no IC₅₀ value was obtained.

Incubation of the cells with PSMA-1-MMAE-IR700 in the dark for 72 h showed that it was 50-fold more effective for killing PC3pip cells than for PC3flu cells. Our results suggest that PSMA-1-MMAE-IR700 selectively delivers MMAE to PC3pip cells leading to effective cell death. To test if combination of MMAE-based chemotherapy and PDT using PSMA-1-MMAE-IR700 would enhance the cytotoxicity, PC3pip and PC3flu cells were incubated with 5 nM of different targeted agents for 24 h, and then cells were washed and treated with or without light. PSMA-1-MMAE-IR700 with light treatment (1 J/cm2 and 3 J/cm2) significantly enhanced the cytotoxicity to PC3pip cells as compared to PSMA-1-IR700 with light treatment and compared to PSMA-1-MMAE-IR700 without light treatment (0 J/cm2) (FIG. 8B). In addition, the cytotoxicity of PSMA-1-MMAE-IR700 increased when the dose of light was increased from 1 J/cm₂ to 3 J/cm₂. CDI was 0.423/(0.778×0.892)=0.609 and 0.208/(0.424×0.892)=0.550 at 1 J/cm₂ and 3 J/cm₂ light irradiation, respectively, suggesting that combination of PSMA-targeted PDT and MMAE caused synergistic effects. In contrast to the cytotoxicity observed in PC3pip cells, no effective cell killing was observed in PC3flu cells when the cells were treated with 5 nM of PSMA-1-MMAE-IR700 or PSMA-1-IR700 with or without light, indicating the cell killing effect is selective to PSMA expression.

### In Vivo Fluorescence Imaging Results

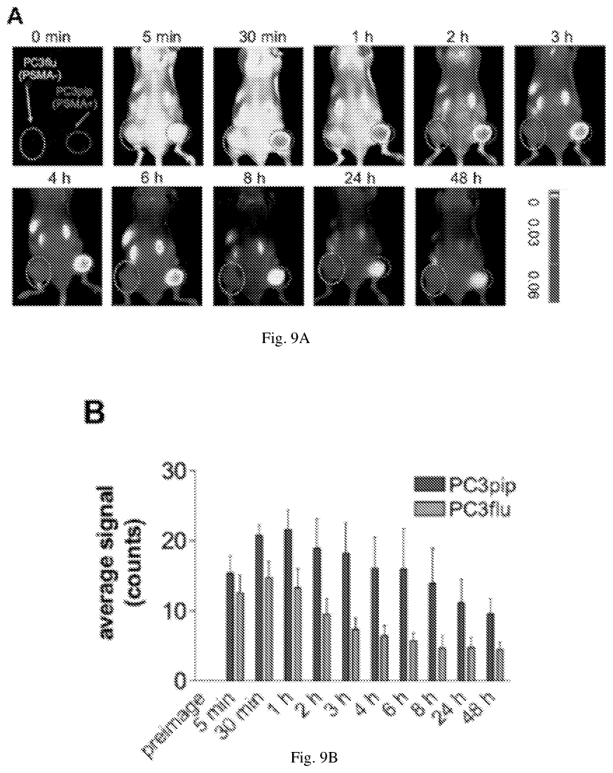

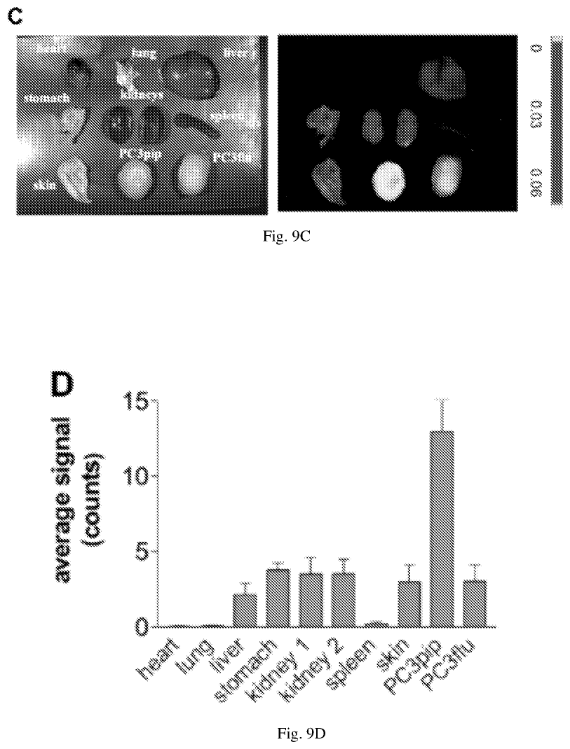

To evaluate the selectivity of PSMA-1-MMAE-IR700, in vivo fluorescence imaging was performed in mice bearing both PC3pip and PC3flu tumors. PSMA-1-MMAE-IR700 (100 nmol/kg) was administered through tail vein injection, and mice were imaged at various time points. As shown in FIGS. 9A-B, selective uptake was observed in PC3pip tumors. The time to reach peak uptake in PC3pip tumors was 1 h post injection. At 4 h post injection, the fluorescence signal in PC3 pip tumor was 3.8-fold higher (19.6±4.4 counts) than that in PC3flu tumor (5.2±1.5 counts); at 48 h post injection, the difference between the fluorescence signal from the two tumors was 2.9-fold (9.6±2.1 counts on PC3pip tumors vs 3.3±1.3 counts on PC3flu tumors) (FIG. 9B). Ex vivo imaging at 48 h post injection showed that fluorescence was mainly retained in the PSMA-positive PC3pip tumor (FIGS. 9C-D). Minimal fluorescence was observed in PSMA-negative PC3flu tumor, liver, spleen, lung, kidneys, heart, lung, skin, and stomach.

The orthotopic PC3pip prostate cancer mouse model can develop tumor metastases to lymph nodes. To test if PSMA-1-MMAE-IR700 can detect lymph node metastases, we performed fluorescence imaging in mice bearing PC3pipGFP tumors. Twenty-one days following orthotopic implantation of PC3pip cells into the prostate gland, mice received 100 nmol/kg PSMA-1-MMAE-IR700. At 1 hour post injection (peak tumor accumulation time determined above), bright fluorescence signal from PSMA-1-MMAE-IR700 was observed in primary orthotopic PC3pipGFP tumors and the signal correlated with the GFP fluorescence signal in the tumor (FIG. 10A). Removal of the primary tumor allowed visualization of enlarged lymph nodes. GFP signal in the lymph nodes proved presence of tumor in the lymph nodes. Fluorescence signal from PSMA-1-MMAE-IR700 corresponded to the GFP in the lymph nodes. Ex vivo imaging of tissues at 1 h post injection showed that PSMA-1-MMAE-IR700 fluorescence was only observed in the tumor and kidneys. The signal in kidneys dropped much more rapidly from 1 h to 48 h (FIGS. 9C-D) as compared to the signals in PC3pip tumors, suggesting that the signal in kidneys was mainly due to renal clearance of PSMA-1-MMAE-IR700. Further histological analysis was performed in the primary tumor and lymph node (FIG. 10B). Fluorescence signal from PSMA-1-MMAE-IR700 was only observed in cancer tissues, but not in normal prostate or normal lymphocytes. PSMA-1-MMAE-IR700 fluorescence signal was highly co-localized to GFP signal from the tumor. In addition, PSMA-1-MMAE-IR700 was able to define the borders between the cancer tissue and normal tissue.

### In Vivo Antitumor Activity Studies

The effectiveness of PSMA-1-MMAE-IR700 to eliminate prostate tumors was performed in mice bearing PC3pip tumors. Mice received 100 nmol of PSMA-1-MMAE-IR700 through tail vein injection every 4 days with a total of 5 doses. Mice were irradiated by 50 J/cm² of 690 nm light at the peak tumor accumulation of PSMA-1-MMAE-IR700, which was 1 h post injection. Controls included i.v. administration of PBS with no light treatment, 100 nmol/kg of PSMA-1-MMAE-IR700 with 50 J/cm² of 690 nm light, 100 nmol/kg of PSMA-1-MMAE-IR700 with no light treatment, 100 nmol/kg of PSMA-1-IR700 with 50 J/cm² of 690 nm light, 100 nmol of PSMA-1-IR700 with no light treatment, co-injection of 100 nmol/kg of PSMA-1-IR700 and 100 nmol/kg of free MMAE with 50 J/cm² of 690 nm light, and co-injection of 100 nmol/kg of PSMA-1-IR700 and 100 nmol/kg of free MMAE with no light treatment. As shown in FIGS. 11A-B, PSMA-1-IR700 with no light and PSMA-1-IR700+MMAE with no light failed to inhibit tumor growth and extend animal survival. In contrast to the group that received PSMA-1-IR700+MMAE without irradiation, PSMA-1-IR700 with light irradiation and PSMA-1-IR700+MMAE with light irradiation showed the ability to inhibit tumor growth. PDT caused tumor swelling in some mice. Most of the mice in these two PDT groups initially displayed tumor growth inhibition, but after the treatment period the tumor started to grow again leading to animal death, with one mouse in the PSMA-1-IR700+PDT group dying on day 32 of the treatment (FIGS. 11A-B). These data indicate that PDT alone is not enough to kill the tumors completely. PSMA-1-MMAE-IR700 without light significantly inhibited tumor growth and prolonged animal survival, indicating that targeted delivery of MMAE improved antitumor activity. The greatest tumor growth inhibition was found in the animals receiving PSMA-1-MMAE-IR700 with light irradiation. During PSMA-1-MMAE-IR700+PDT treatment, initial tumor swelling was observed in some mice, resulting in slightly larger initial tumor sizes than those measured after treatment with PSMA-1-MMAE-IR700 without PDT. Importantly, the sizes of PSMA-1-MMAE-IR700+PDT treated tumors continued to shrink even when the treatment ceased. In contrast, the tumors of the mice receiving PSMA-1-MMAE-IR700 without PDT treatment started to grow when the treatment stopped. Between day 40 to day 60, all 5 mice that received PSMA-1-MMAE-IR700 with PDT were tumor free. After day 60, tumor started to grow back on 2 of the mice, and the other 3 mice remained tumor free during the 90-day experimental period. Although we were not able to obtain the CDI values as the control mice started to die as early as on day 18, our data demonstrated that PSMA-1-MMAE-IR700+PDT significantly inhibited tumor growth (FIG. 11A) and extended animal survival (FIG. 7B) as compared to the other 6 groups. Therefore, PSMA-1-MMAE-IR700 with light improved the treatment outcome of PSMA-1-IR700 with light irradiation and PSMA-1-MMAE-IR700 with no light irradiation. In addition, the treatment did not cause any body weight loss (FIG. 11C). Further histological analysis of major organs in mice treated with PSMA-1-MMAE-IR700 on day 30 did not show any considerable microscopic changes in major organs, such as liver, lung, heart, spleen, kidney, salivary gland and prostate, indicating the treatment did not cause systemic toxicity.

### Induction of Apoptosis by Treatment

To evaluate the apoptosis caused by the treatment, mice bearing PC3pip tumors were treated with drugs and tumors were collected four days after the treatment, sectioned, and examined by H&E and caspase 3 assay (FIG. 11D). The histology of the tumors treated with PSMA-1-IR700 without PDT and PSMA-1-IR700+MMAE without PDT remained the same as the PBS-treated control tumors. No immunofluorescence signal related to apoptosis was observed in these tumors. Treatment of the tumors with PSMA-1-IR700 with PDT or PSMA-1-IR700+MMAE with PDT caused significant necrotic damage (H&E) to the tumor tissue. The Caspase 3 assay revealed large portion of apoptotic lesions in the tumors. Similar observations were found in the tumors treated with PSMA-1-MMAE-IR700 without PDT. The most aggressive necrotic damage and apoptosis was observed in tumors receiving PSMA-1-MMAE-IR700 with PDT, suggesting that combination of targeted PDT with targeted chemotherapy enhanced the treatment effect.

Prostate cancer is highly heterogeneous, which will affect treatment response, drug resistance and clinical outcome. The use of combination therapies with different mechanisms of action will offer potential advantages over a single therapy and it can be an effective way to deal with the ‘heterogeneity’ of cancer cells. However, this is not a simple approach because different drugs may have different pharmacokinetics and do not necessarily get to the tumor at the same time and the drug can also go to other tissues in the body causing side effects. The use of anticancer drugs is therefore limited by unwanted side effects. To overcome these problems, we have developed a multifunctional molecule named PSMA-1-MMAE-IR700 that combines chemotherapy, PDT, and imaging in a single molecule that is targeted to PSMA. PSMA is over expressed almost exclusively on prostate cancer (FIG. 6A). MMAE was selected as the chemotherapeutic drug because of its high potency, its wide use in antibody drug conjugates, its synergy with PDT and the fact that we have successfully targeted it to PSMA for the treatment of prostate cancer with a greater therapeutic index when compared to a PSMA-targeted antibody drug conjugate using MMAE. IR700 was selected as the photosensitizer due to its good water-solubility, its high fluorescence quantum yield and singlet oxygen yield, its long wavelength absorption (690 nm), and the fact that a cetuximab-IR700 conjugate (RM-1929) has been approved in Japan for local regional treatment of head and neck cancer. Conjugation of MMAE and IR700 to the PSMA-1 ligand did not reduce the binding affinity of PSMA-1-MMAE-IR700 to PSMA (FIG. 10C). Stability studies showed that while PSMA-1-MMAE-IR700 was stable in PBS, it degraded in mouse plasma with a half-life at 17.2±2.8 h. The fluorescence from IR700 allowed visualization of uptake studies of PSMA-1-MMAE-IR700. PSMA-1-MMAE-IR700 showed selective uptake in PSMA-positive PC3pip cells, but not in PSMA-negative PC3flu cells, and its binding was specific to PSMA receptor on the cells as indicated by the blocking experiment with excess PSMA-1 ligand (FIG. 7). Once it entered the cells, PSMA-1-MMAE-IR700 was almost exclusively located in the lysosomal compartment. These results are consistent with our previous studies. In in vitro cytotoxicity studies, PSMA-1-MMAE-IR700 without light activation selectively killed PC3pip cells, likely due to protease release of MMAE, which does not require PDT (FIG. 8). Furthermore, PSMA-1-MMAE-IR700 with light treatment showed PDT efficacy and greater cytotoxicity than PSMA-1-MMAE-IR700 without light and PSMA-1-IR700 with light. These data suggested that the combination of PDT and MMAE in a single molecule was the most effective at killing PSMA-positive cancer cells. The combination appeared to be synergistic and CDIs of <0.6 were observed. In vivo biodistribution studies showed that PSMA-1-MMAE-IR700 preferentially accumulated in PC3pip tumors (FIG. 9). At 4 h post injection, the fluorescent signal in PC3pip tumors was 3.8-fold higher than in PC3flu tumors. Ex vivo images at 48 h post injection showed that the fluorescent signal in PC3pip tumor was more than 3-fold higher than in kidneys, liver and other organs, which indicates low off-target accumulation of PSMA-1-MMAE-IR700. Pharmacokinetic studies based on the fluorescence of PSMA-1-MMAE-IR700 showed that PSMA-1-MMAE-IR700 cleared quickly from the blood, after 4 h post injection almost all drug was cleared. Although PSMA-1-MMAE-IR700 degraded in mouse plasma, its degradation is much slower than its clearance from the blood stream, therefore, there shouldn't be concerns about toxicity from released MMAE. In fact, no body weight loss nor damage to major organs were observed after the treatment (FIG. 11C). In in vivo antitumor activity studies, PSMA-1-MMAE-IR700 demonstrated significantly greater efficacy for PC3pip tumors than single drug treatments (PSMA-1-MMAE-IR700 without PDT or PSMA-1-IR700 with PDT) (FIG. 11). More noteworthy, PSMA-1-MMAE-IR700 showed improved antitumor activity compared to co-administration of individual drugs (PSMA-1-IR700+MMAE with PDT), addressing the importance of simultaneous drug delivery to cancers. PSMA-1-MMAE-IR700 at 100 nmol/kg with light irradiation resulted in a 60% cure rate for tumors. In previous studies, to reach 60% cure, a dose of 3820 nmol/kg of PSMA-1-VcMMAE was used, therefore, combination of PDT and chemotherapy significantly reduced the dose of chemotherapy required. A caspase 3 assay showed that tumors treated with PSMA-1-MMAE-IR700+PDT had more apoptosis than tumors in the other treatment groups (FIG. 11D), indicating that PSMA-1-MMAE-IR700+PDT is the most efficacious treatment. It is speculated that PSMA-1-MMAE-IR700+PDT may kill significantly more cancer cells than other treatment groups, leading to slower regrowth, i.e., less residual tumor cells, and/or therapeutic cures.

Our molecule is the first example that combines both chemotherapy and PDT in a single targeted small molecule for a dual-therapeutic approach to combat prostate cancer. The selective targeting and rapid clearance of the molecule should dramatically reduce off-target toxicity while simultaneously increasing anti-cancer efficacy. For localized prostate cancer, minimally invasive fiber optics have been developed to irradiate the prostate gland with light, e.g., TOOKAD, providing the needed infrastructure for implementation of the PDT approach. In addition to localized NIR light irradiation, the efficacy of PSMA-1-MMAE-IR700 can be extended to systemic cell killing by local release of active MMAE, which will overcome the problem that PDT cannot be used to treat large tumors due to limited light penetration. On the other hand, PDT will reduce the needed dose of the chemotherapeutic drug, therefore further reducing dose-related toxicity of MMAE. Compared to current clinical protocols for combination therapy of PDT and chemotherapy, our approach selectively delivers both drugs to cancer cells, reducing off-target toxicity related to untargeted drugs and achieving enhanced synergistic antitumor activity. Furthermore, the PDT agent, IR700, emits light at 700 nm when irradiated by 690 nm light. We have demonstrated that PSMA-1-MMAE-IR700 can identify cancer tissues, including metastases to lymph nodes, and delineate tumor margins (FIG. 10), which further expands its use for fluorescence imaging and image-guided surgery for prostate cancer. It has been reported that IR700 can be imaged by a clinically available imaging instrument, LIGHTVISION (Shimaduzu, Japan). Therefore, PSMA-1-MMAE-IR700 can help surgeons visualize the tumor and resect tumors during surgery. We have demonstrated that PDT is an effective adjuvant therapy after fluorescence image guided surgery (FIGS). In the case of PSMA-1-MMAE-IR700, it can offer both adjuvant PDT and chemotherapy FIGS to eradicate any unresected cancer cells, resulting in complete tumor removal. PSMA-1-MMAE-IR700 showed the ability to accumulate in tumor metastases in lymph nodes, which indicates that the drug may be effective in combating tumor metastases. In addition, both MMAE and PDT have been reported to initiate a post-treatment immune response against tumor cells. The immune stimulation by our approach may further prevent the metastasis of the disease. Future works are needed to validate the use of PSMA-1-MMAE-IR700 for FIGS and the immune response caused by PSMA-1-MMAE-IR700 treatment using an immunocompetent mouse model of prostate cancer that overexpresses PSMA.

In conclusion, we have synthesized a multi-functional theranostic molecule for simultaneous and targeted delivery of both PDT and chemotherapy to prostate cancer cells. The multifunctional molecule showed selective uptake in PSMA-positive tumors and significantly enhanced (synergistic) antitumor activity was observed as compared to individual treatment with PDT or chemotherapy alone. It can be used in the operating room to help surgeons detect tumors using real-time FIGS, and provide PDT and chemotherapy to kill any unresected cancer cells. It is also possible that the molecule can be used directly on prostate cancer patients that are not suitable for surgery, providing PDT and chemotherapy to cancer tissues.

### Example 3

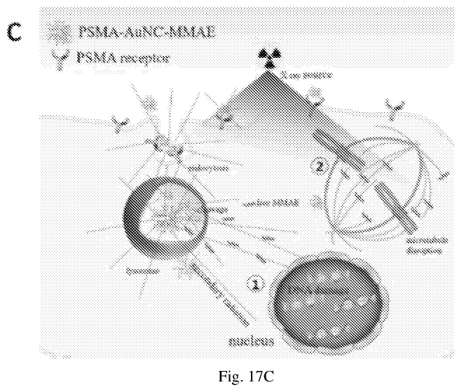

This example describes theranostic gold nanoclusters as prostate cancer (PCa) targeted radiosensitizers and chemotherapy delivery vehicles. We designed a peptide-prodrug conjugate, PSMA-MMAE, with protease-based MMAE release and used it to synthesize PCa-targeted PSMA-AuNC-MMAE having a renal-clearable size to reduce potential toxicity (FIG. 17). PSMA targeting enhanced the specificity of cellular uptake. MMAE release was governed by cathepsin activity in the tumor. The PSMA-AuNC-MMAE enabled significant amplification of radiation efficacy for PCa in vitro and in vivo and potentially will improve the efficacy and therapeutic index of MMAE and RT for PCa.

### Methods

### Synthesis of CY-PSMA-MMAE-SH Ligands

PSMA targeting peptide Glu-CO-Glu′-Amc-Ahx-Glu-Glu-Glu-Lys-Cys (CY-PSMA-1) was synthesized by Fmoc chemistry as described previously. C(Trt)Y-PSMA-1 was also synthesized to keep the —SH protected by Trt. All the amino acids were purchased from Peptides International Inc. The synthesized CY-PSMA-1 ligands were purified by a HPLC system (Shimadzu) with Luna 5m C18 semipreparative column and characterized by electrospray ionization mass spectrometry (ESI-MS, LCQ advantage, Thermo Finnigan).

The PSMA targeted prodrug, CY-PSMA-MMAE, was synthesized by conjugating monomethyl auristatin E (MMAE) to the Cys residue of CY-PSMA-1 ligands via a maleimido caproyl valine-citrulline (Vc) cathepsin-cleavable linker with a self-immolative p-aminobenzyl carbamate (PABC) spacer. Briefly, 1.5 equiv of MMAE (BOC Sci.) was dissolved in DMF and added to 1 equiv of CY-PSMA-1 peptides in DMF. The pH of reacting mixture was then adjusted to above 7.5 by using diisopropylethylamine (DIPEA) and stirred at room temperature for over 2 h. Purification was performed by using HPLC, and conjugates were confirmed by ESI-MS.

The synthesized CY-PSMA-MMAE or C(Trt)Y-PSMA-1 was reacted with N-succinimidyl S-acetylthioacetate (Pierce SATA), producing CY-PSMA-MMAE-SATA or C(Trt)Y-PSMA-SATA. Generally, 1.5 equiv of SATA was dissolved in DMF and added to 1 equiv of peptide conjugates. The pH of the reaction mixture was adjusted to around 8 by using trimethylamine (TEA) and incubated at room temperature for over 1 h. The final product was purified by HPLC, and the mass was confirmed by ESI-MS.

The purified CY-PSMA-MMAE-SATA or C(Trt)Y-PSMA-SATA was dissolved in DMF and added with 0.5 M hydroxylamine to deacetylate the SATA protection. The pH of mixture was adjusted to around 7.5 by using TEA and stirred at room temperature over 2 h. The final product, CY-PSMA-MMAE-SH or C(Trt)Y-PSMA-SH, was purified by HPLC and characterized by ESI-MS. The detailed reaction is shown schematically in the Supporting Information as well as the MS spectra of product from each step.

### Synthesis of PSMA-AuNC-MMAE and PSMA-AuNCs Conjugates and Characterization

PSMA-AuNC-MMAE conjugates were synthesized by using a one-pot reaction according to a previously reported protocol with slight modifications in terms of pH, reacting temperature and time, and solvent. In a typical synthesis of the NCs, CY-PSMA-MMAE-SH or C(Trt)Y-PSMA-SH was dissolved in 2 mL of PBS with a final concentration of 1 mM. During vigorous stirring, 100 μL of 25 mM HAuCl₄ solution was added to the prodrug conjugates slowly. After about 2 min, 1 M NaOH solution was added to bring the reaction pH up to 12. The mixture was then kept at 37° C. to allow the reaction to proceed for 24 h. The formed NCs were purified, and excess peptides were removed by dialysis against 25 mM HEPES buffer for over 2 h and then against 12.5 mM HEPES buffer for 3 days. The final PSMA-AuNC-MMAE conjugates were lyophilized and stored at −20° C. until further use. PSMA-AuNCs without MMAE were also synthesized by using the same procedure with C(SH)Y-PSMA-1 ligands. PSMA-AuNC-MMAE conjugates were characterized by transmission electron microscope (TEM, FEI Tecnai F300 kV) and a dynamic light scattering system (DynoPro NanoStar). The optical properties (absorbance, excitation, emission) of NCs were measured by Tecan Infinite M200 plate reader and IVIS Spectrum (PerkinElmer).

### MMAE Release Assay

Cathepsin B (human liver, Athens Research & Technology) was activated by using a protocol as described previously. PSMA-AuNC-MMAE conjugates were then added to 500 μL of activated cathepsin B buffer, and the mixture was stored at 37° C. At the time points of 1, 7, 24, and 48 h, a 10 μL solution was taken out for HPLC analysis. As a control, 1 mM E64 protease inhibitor was added to the PSMA-AuNC-MMAE conjugates, and the same procedure and analysis was carried out. The cleaved free MMAE from NCs was determined by the HPLC system equipped with a Luna 5 m C18 analytical column and SPD-20A prominence UV/vis detector at 220 and 254 nm. The amount of free MMAE was calculated according to an established calibration curve.

### Selective Cell Uptake and Cytotoxicity

The cell uptake of PSMA-AuNC-MMAE conjugates was evaluated by using human prostate cancer cell lines: PC3pip cells (PSMA+) which were retrovirally transformed with PSMA and PC3flu cells (PSMA-) as a transformation control (Laboratory of Gene Transfer and Gene Expression, Gene Transfer and Somatic Cell Engineering Facility, Memorial-Sloan Kettering Cancer Center). Both cells were cultured in RPMI1640 medium with L-glutamine (2 mmol/L) and 10% FBS at 37° C. and 5% CO₂. To image the uptake of PSMA-AuNC-MMAE conjugates and disruption of α-tubulin, PC3pip and PC3flu cells were seeded in 8-well μ-slide (ibidi GmbH). When the cells growth reached 70% confluence, PSMA-AuNC-MMAE conjugates were added at calculated MMAE concentration of 5 nM and incubated for 1, 4, and 24 h at 37° C. Another group of cells were pretreated with 50 μM E64 inhibitor before adding the PSMA-AuNCs-MMAE. Cells were then washed with PBS, fixed with 4% paraformaldehyde for 10 min, permeabilized by 0.1% Triton X-100 for 10 min, and blocked by 1% BSA for 1 h at 37° C. Then both PC3pip and PC3flu cells were incubated with 2 μg/mL Alpha-tubulin (B5-1-2) Alexa Fluor 488 Mouse Monoclonal Antibody (Invitrogen) in 0.1% BSA at 37° C. for 3 h. Cells were then washed and counterstained with DAPI for 10 min before observation by using a Leica HyVolution SP8 confocal microscope (Leica Microsystem Inc.).

In vitro cytotoxicity was measured by MTT assay. Cells (10000 cells/well) were seeded in 96-well plates and incubated for 24 h before adding 5 nM free MMAE, PSMA-MMAE, or PSMA-AuNCs-MMAE. An equivalent amount of PSMA-AuNCs without MMAE was added as a control, and another group of cells pretreated with 50 μM E64 inhibitor for 6 h was also incubated with PSMA-AuNCs-MMAE. After incubation for 1, 4, and 24 h, cells were rinsed with PBS and incubated with 1 mg/mL MTT solution in culture media for 3.5 h. After that media were removed, and 100 μL of DMSO was added. The absorbance was measured 15 min later by a plate reader (Tecan Infinite M200) with absorbance at 570 nm and reference at 620 nm.

### In Vitro Radiotherapy

Both PC3pip and PC3flu cells (1×10⁵ cells/well) were cultured in 6-well plates for 24 h, and then PSMA-AuNC-MMAE and PSMA-MMAE were added at an equivalent MMAE concentration of 1 nM. Additionally, PSMA-AuNCs without MMAE was added at 60 μg Au/mL. Another group with the addition of a mixture of PSMA-AuNCs and PSMA-AuNC-MMAE at the equivalent dose of Au and MMAE was also evaluated. Mixing was performed so as to keep Au concentrations equivalent. This could not be achieved by adding more PSMA-AuNC-MMAE as this would result in toxic levels of MMAE. Cells without any treatment were used as blank control. Following incubation for 6 h, the media were removed, and cells were rinsed by PBS before exposing to X-ray radiation at doses of 0, 2, 4, and 6 Gy. Next, cells were trypsinized, counted, and reseeded in 6-well plate for colonies to grow. After 2 weeks, all the wells were rinsed with PBS, fixed with 4% paraformaldehyde, and stained by 0.4% crystal violet. The number of colonies from each well was counted to calculate the survive fraction.

### In Vivo Tumor Targeting and Biodistribution

Under the guidelines of the animal care and use committee at Case Western Reserve University (IACUC #150033), 4- to 5-week-old male athymic nude mice were subcutaneously implanted with PC3pip or PC3flu cells (100 μL matrigel containing 1×10⁶ cells) on the right flank. When the tumors reached a sufficient size, mice were intravenously injected with PSMA-AuNCs (15 μg/g) and imaged by using the IVIS Spectrum system before and at 0.5, 1, 2, 4, 6, and 24 h postinjection (excitation filter at 500 nm and emission filter at 680-800 nm). Unmixing was performed by using the in vivo spectrum of PSMA-AuNCs to remove the autofluorescence background. All the images at different time points were demonstrated at the same optical threshold. The tumor region was determined in a free-drawn manner guided by the bright-field image, and average radiant efficiency (photos s⁻¹ cm⁻¹ sr⁻¹ (μW cm⁻²)⁻¹) was calculated for each time point. At 24 h post injection, mice were euthanized, and tumor/organs were extracted for ex vivo imaging using the same setting, and the average radiant efficiency for all the tissues was calculated. The organs were also digested and analyzed by ICP-MS. Each group of treatment was done in triplicate.

### In Vivo Chemoradiotherapy

Male athymic nude mice were subcutaneously implanted with the PC3pip tumor, and when the tumor size reached about 100 mm³ (tumor volume=length×width²/2), the mice were divided into ten groups randomly. Each group was intravenously injected with PBS, PSMA-AuNCs, PSMA-AuNCs-MMAE, PSMA-MMAE, or a mixture of PSMA-AuNCs and PSMA-MMAE at MMAE dose of 400 nmol/kg and an equivalent dose of Au. At 4 h postinjection, five of the groups were irradiated with X-rays (6 Gy, Cs-137 with energy of 0.6616 MeV) focusing on the tumor region, and the other five groups received the same injections but no radiation and were used as controls. Each group contained five mice. All the treatments were given only once, and then the mice were monitored for tumor size and body weight every other day for one month. The coefficient of drug interaction (CDI) is calculated as follows: CDI=C/(A×B),⁴⁴ where C is the tumor volume ratio of PSMA-AuNCs-MMAE+RT group to control group, A is the tumor volume ratio of PSMA-AuNCs+RT group to control group, and B is the tumor volume ratio of PSMA-MMAE+RT group to control group; PBS+RT group is the control. A CDI value of <1, =1, or >1 indicates that the treatments are synergistic, additive, or antagonistic, respectively. When the tumors became too large or an animal was moribund, they were euthanized. At the end of monitoring, all mice were euthanized and tumors were isolated and weighed.

### Histology, Detection of AuNPs, and Immunofluorescent Analysis

Groups of mice injected with PBS, PSMA-AuNCs, PSMA-AuNCs-MMAE, PSMA-MMAE, and a mixture of PSMA-AuNCs and PSMA-MMAE were euthanized at 24 h postinjection. Groups of mice that received radiation were euthanized 1 h after irradiation. All tumor samples were snap-frozen in optimum cutting temperature compound (OCT) for cryosectioning (Leica CM3050S). Sections, 12 μm thick, were serially collected directly onto slides and stored at −80° C. for processing. Adjacent slides were stained for presence of NCs with a silver staining assay (Sigma Silver Enhancer Kit) followed by H&E standard procedures. α-Tubulin of tumor samples was stained with a-tubulin (B5-1-2) Alexa Fluor 488 Mouse Monoclonal Antibody (Invitrogen) by using the sample protocol as described in the in vitro uptake section. Tissue nuclei were stained with DAPI and tumor tissues were imaged by Leica HyVolution SP8 confocal microscope (Leica Microsystem Inc.).

For immune-histochemical analysis (IHC), the slides were warmed to room temperature for 10 min, fixed with 10% buffered formalin, blocked in blocking buffer (5% normal goat serum/0.3% Triton X-100 in 1×PBS) for 1 h at room temperature, and incubated in primary antibody for 1-3 h followed by three 5 min washes in 1×PBS. The presence of apoptosis in the tumor was evaluated by rabbit anti-Cleaved Caspase-3 antibody (#700182, Cell Signal Tech) at a 1:400 dilution. The presence of the histones with double-strand breaks in DNA of the tumor nuclei was evaluated by rabbit anti-yH2AX (phospho-Ser139) antibody (SAB5600038, Sigma-Aldrich) at a 1:500 dilution. After washing, the slides were treated with the secondary ready-to-use antibody (goat anti-rabbit polyclonal antibody labeled by Alexa Fluor-594 (Invitrogen, Inc.)) for 20 min at room temperature followed by double washing with PBS for 5 min. Tissue nuclei were contrasted by using Fluoro-Gel-II with DAPI (Electron Microscopy Sciences, Hatfield, PA). Fluorescent images were viewed with a Leica-DM4000B microscope (bandpass=560/645, for Cleaved Caspase-3 or yH2AX) and analyzed with QCapturePro-7 software. An Olympus-VS120/S5 versatile microscope-based scanner was used to generate histological images larger than a single field of view.

### Statistical Analysis

All numerical results are expressed as mean±SD. Descriptive statistics and significant differences between groups were analyzed by using two-tailed Student's t tests, and the difference was considered significant if *p<0.05 and **p<0.01.

### Results

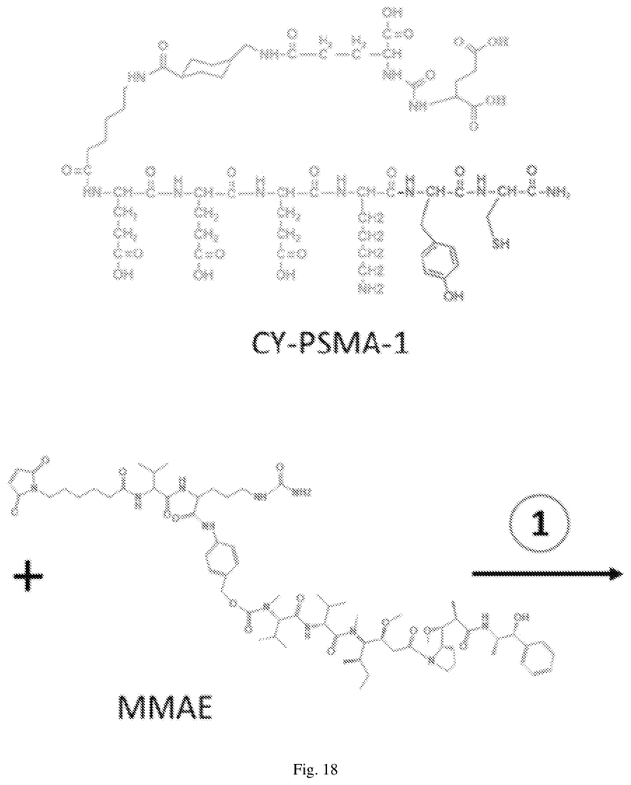

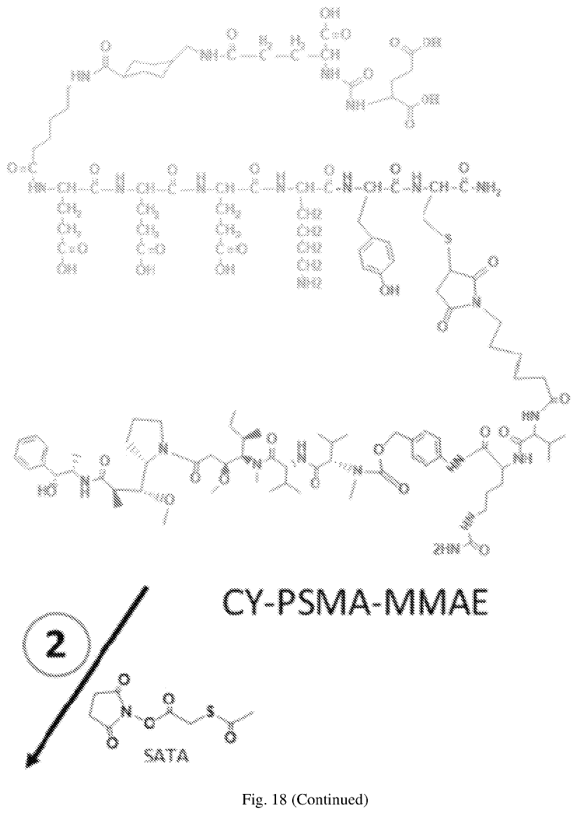

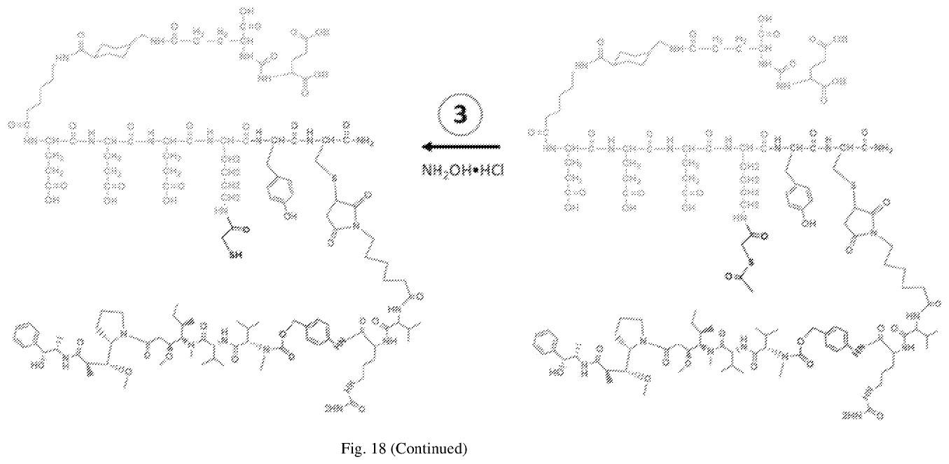

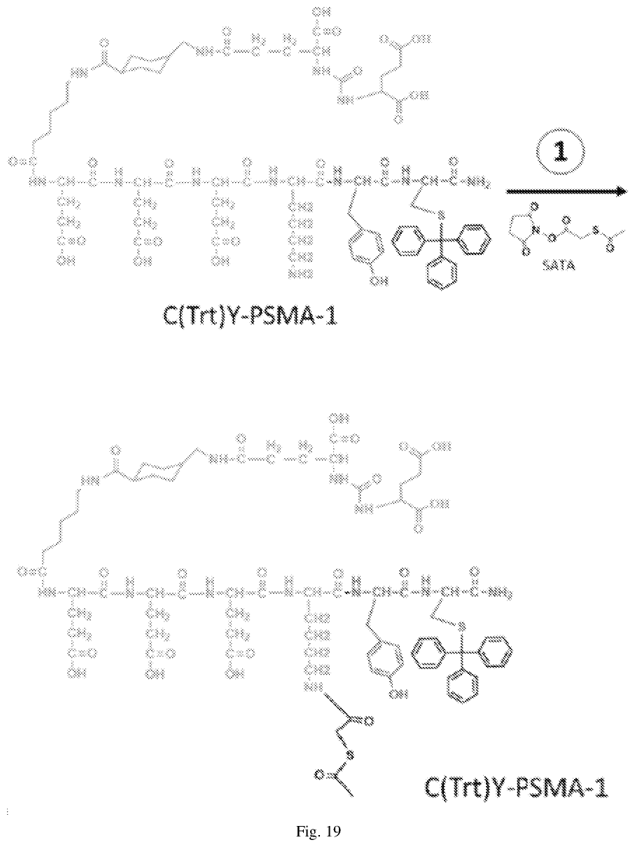

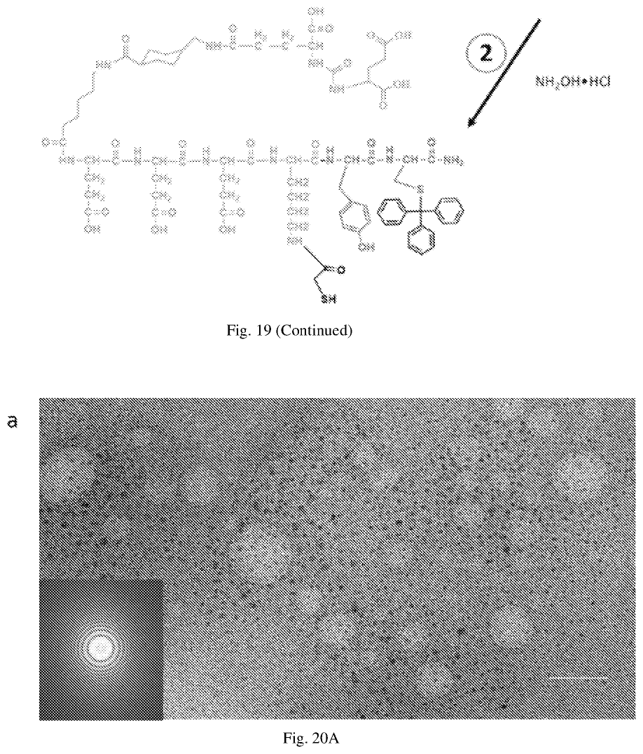

PSMA-AuNC-MMAE conjugates were synthesized by using PSMA-MMAE as a template in a one-step reaction (FIG. 17). Our first step was to conjugate the MMAE to the PSMA-1 ligands via a clinically approved cathepsin B sensitive linker, maleimidocaproyl-valine-citrulline-p-aminobenzyl carbamyl (MC-VC-PABC) (FIG. 17A). To enable successful formation of NCs, we then modified the peptide-MMAE conjugates with SATA through the Lys residue to induce active —SH groups on the conjugate with two additional synthesis steps (FIG. 18). The Tyr and —SH modified Lys residues reduced Au³⁺ into nanoclusters, AuNCs, anchoring the PSMA-1 targeting moiety and cathepsin B releasable MMAE on their surface (FIG. 17B). Actively targeted peptides have been incorporated into AuNCs with this single step procedure with improved affinity for tumor biomarkers. PSMA-AuNC lacking MMAE conjugation were also synthesized (FIG. 19).

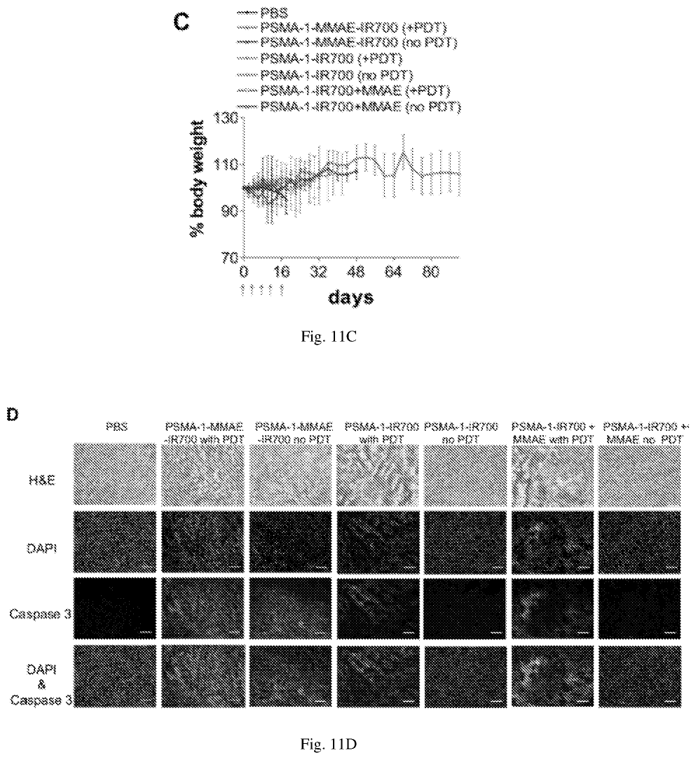

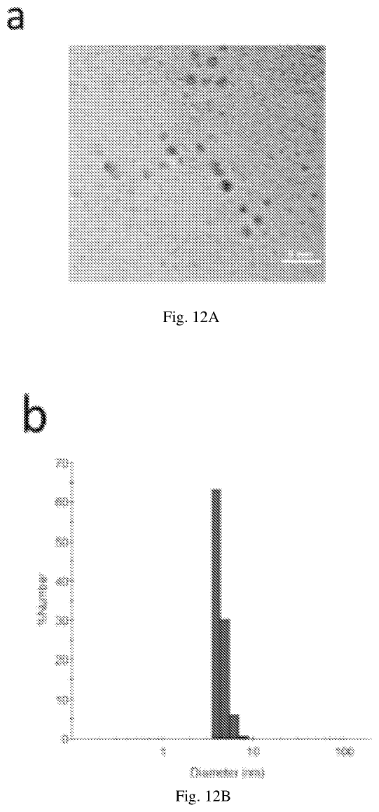

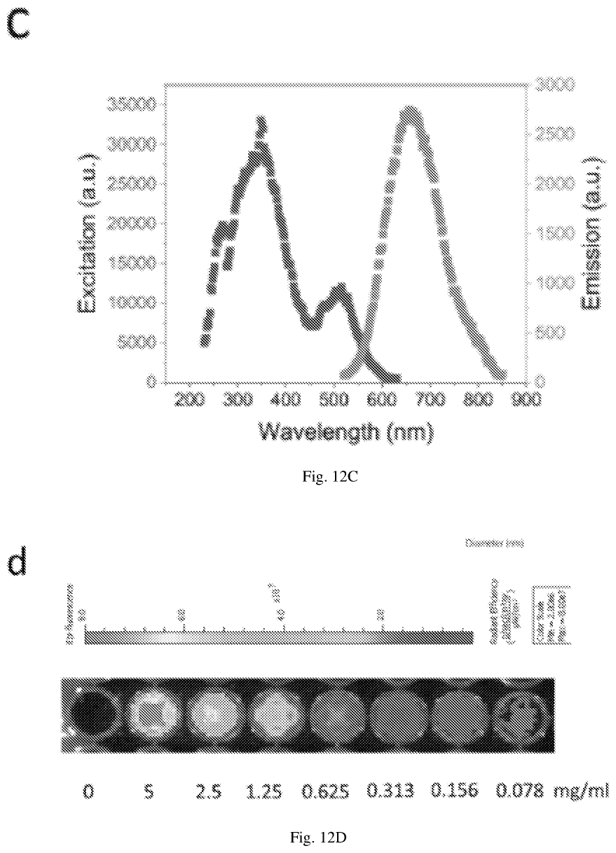

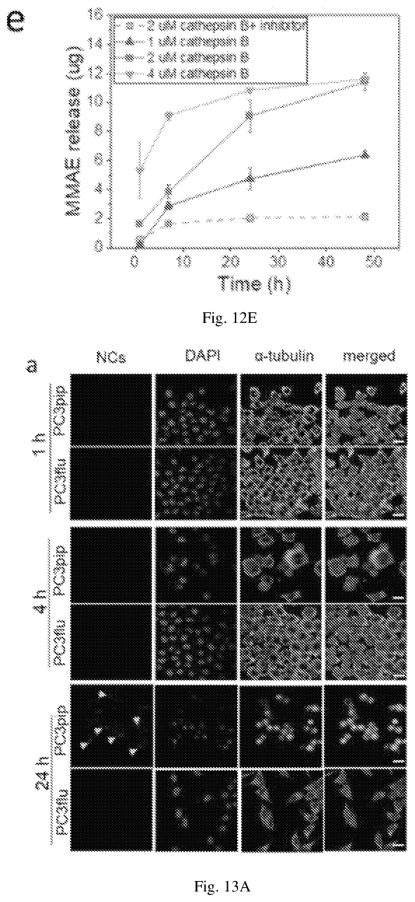

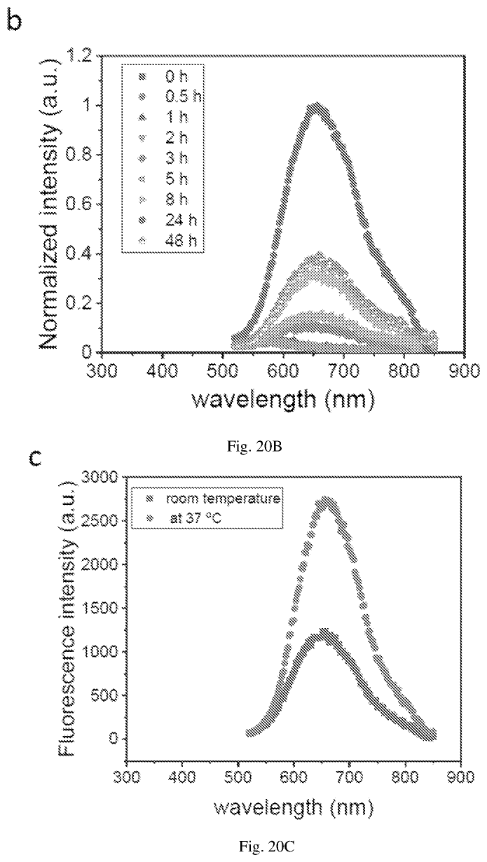

The synthesized PSMA-AuNC-MMAE conjugates had an average core size of 1.2±0.2 nm as revealed by transmission electron microscopy (TEM, FIG. 12A and FIG. 20A) and a hydrodynamic diameter of 4.4 nm (PDI=0.16) (FIG. 12B). An intensive NIR emission in the 600-750 nm range was observed when the PSMA-AuNC-MMAE conjugates were excited at 490 nm (FIG. 12C). The NIR fluorescence peak of 675 nm for PSMA-AuNC-MMAE originates from the interaction between the intrinsic Au core and the surface ligands. Alterations in synthesis procedure can result in slightly different emissions (FIG. 20). To evaluate the fluorescence imaging sensitivity, PSMA-AuNC-MMAE dilutions from 0.078 to 5 mg/mL were imaged by a Spectrum IVIS imager with A_{exc/em} of 490/700 nm. There was a linear relationship between the average radiant efficiency (photons s⁻¹ cm⁻² sr⁻¹ (μW cm⁻²)⁻¹) and the nanocluster concentration (FIG. 12D).

The PSMA-AuNCs show good stability in physiological solutions due to the zwitterionic peptide coating. A previous study of PSMA-AuNCs with no MMAE revealed a structure with a core consisting of 25 Au atoms and 18 PSMA ligands on the surface. We assumed that since MMAE was conjugated to the PMSA ligand, there would also be 18 MMAE drug molecules attached to each nanocluster. To confirm this, we incubated the PSMA-AuNC-MMAE conjugates with excess activated cathepsin B to release free MMAE. After 48 h incubation, 95% of MMAE (11.4 μg) was released from the NCs according to the high performance liquid chromatography (HPLC) measurement, which was very close to the 12 μg of MMAE that theoretically should be attached. When protease inhibitor E64 was added, release of intact MMAE was significantly reduced with only 17.5% MMAE released after 48 h. In addition, the MMAE release was dependent on the concentration of cathepsin B (FIG. 12E).

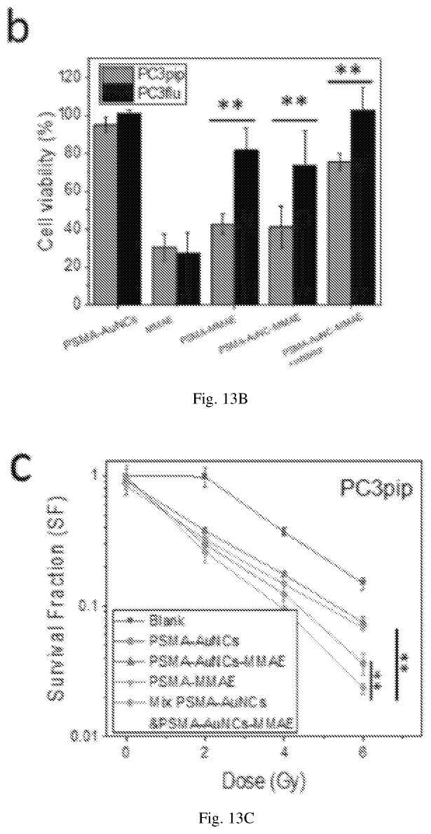

The selective uptake of PSMA-AuNC-MMAE conjugates was evaluated by incubating them with both PSMA-expressing PC3pip cells and PSMA-lacking PC3flu cells. Confocal fluorescence imaging of cells revealed that the PC3pip cells had avid internalization of NCs after 24 h incubation, while the PC3flu cells still showed no evidence of NCs fluorescence (FIG. 13A). Previously, PSMA-targeted particles or small molecular ligands all showed rapid selective and robust intracellular uptake as early as 1 h; however, the weak NCs fluorescence signal here was likely due to the low concentration of PSMA-AuNC-MMAE (5 nM), but there was significant a-tubulin disruption induced by MMAE release intracellularly. At 1 h, a few rounded-up PC3pip cells could be distinguished, and after 4 h, most PC3pip cells had irregular shapes with green fibrous a-tubulin network disruption and loss of spindle structure. At the same time, PC3flu cells still presented similar morphology compared to the blank control cells (FIG. 21). At 24 h, all PC3pip cells were rounded-up with no evidence of fibrous a-tubulin networks, and the nuclei were abnormally shaped. These results would be expected, as MMAE blocks proliferating cells in G₂-M by destabilization of a-tubulin. When MMAE cleavage and release was inhibited by a protease inhibitor, a-tubulin disruption and cytotoxicity could be decreased (FIG. 22). Similar observations were made when MMAE was conjugated to peptides or antibodies, and the activity of MMAE could be completely eliminated by using a noncleavable linker.

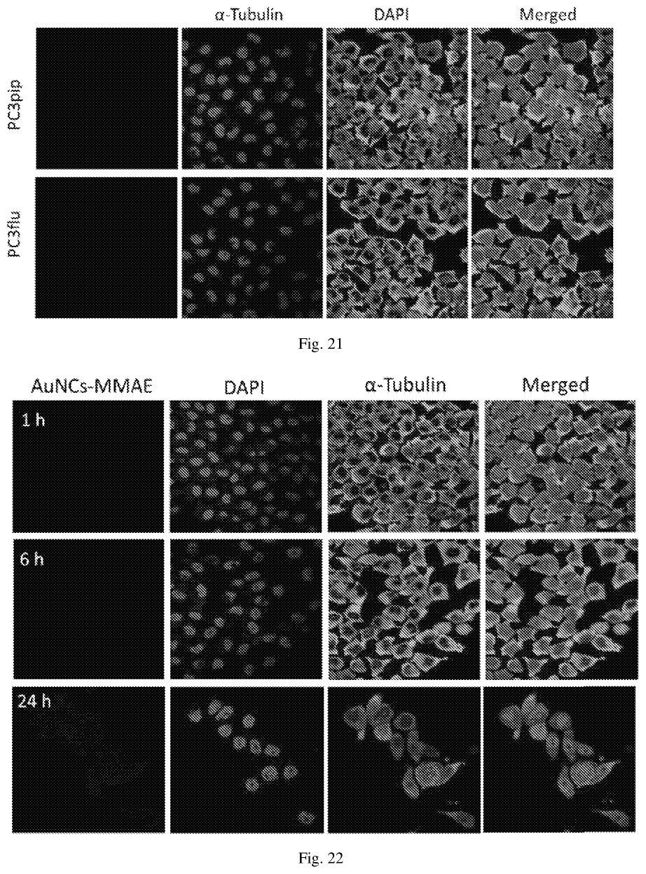

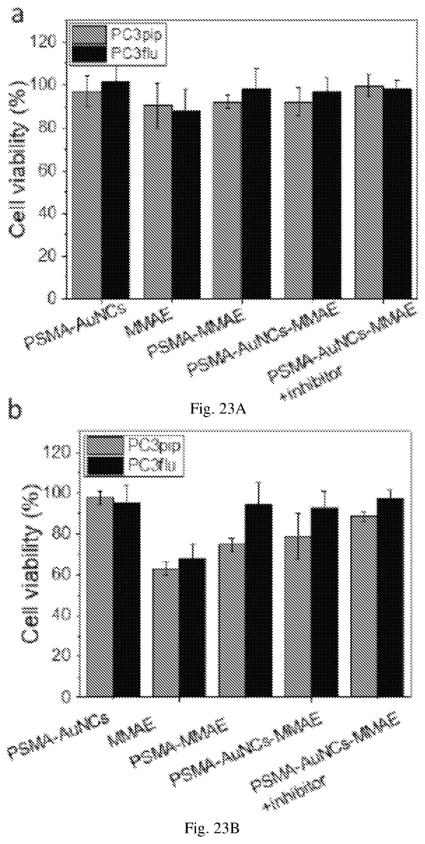

We then measured the cell viability after each treatment. MMAE has been shown to be highly efficatious for killing cancer cells in the form of a free drug or as antibody conjugates. Here, after incubation with PC3pip and PC3flu cells for 24 h (FIG. 13B), the free MMAE (5 nM) nonselectively killed over 70% of cells. When MMAE was conjugated with PSMA-1 ligands, MMAE could be selectively internalized by PC3pip cells, leading to 58% cell killing, while the viability of PC3flu cells only dropped by 18%. PSMA-AuNCs without MMAE showed no toxicity to either cell type. However, after incubation of PC3pip or PC3flu cells with PSMA-AuNC-MMAE, there was selective cell killing comparable to the small molecule PSMA-MMAE molecules. With protease inhibitor E64, cell viability was increased for both cells. The cytotoxicity for cells with the same treatments and incubation of 1 and 4 h is shown in FIG. 23.

We next investigated the radiation enhancement by both MMAE and AuNC conjugates. Au atoms have a high Z number and are well-known for RT sensitization. Au can absorb radiation energy and lead to DNA damage by the photoelectric and Auger effects. MMAE, on the other hand, can also increase radiosensitization by blocking the cells in G₂-M, when cells have the highest sensitivity to radiation. By incubating PC3pip and PC3flu cells with PSMA-AuNCs alone, PSMA-AuNC-MMAE, PSMA-MMAE, and a mixture of PSMA-AuNCs and PSMA-AuNC-MMAE with equivalent Au and MMAE concentrations, we expected to measure a synergistic sensitizing effect. We added the mixture group to achieve optimal effective doses of gold for radiosensitization and of MMAE for chemotherapy, as the MMAE/Au ratio is fixed in the NCs. This approach normalized the amount of MMAE and Au that the cells received. As shown in FIGS. 13C, D, PC3pip cells showed a much lower survival rate compared to the blank control with the same radiation dose, indicating excellent radiosensitization using PSMA-AuNCs (60 μg Au/mL). PC3pip cells incubated with PSMA-MMAE (1 nM) also demonstrated a notably lower survival rate, but there was no significant difference compared to the PSMA-AuNC-MMAE conjugates at the same MMAE dose. This was likely due to the low Au content added to the cells. In contrast, for the mixture of PSMA-AuNCs and PSMA-AuNC-MMAE with the same level of Au (increased to 60 μg/mL) and 1 nM MMAE content, PC3pip cells showed the lowest survival rate, indicating best sensitizing effect by both Au and MMAE. In comparison, similarly treated PC3flu cells showed modest radiation enhancement, but not as dramatic as the effect for PC3pip cells, indicating selective sensitization by targeting agents with PSMA-1 ligands (FIG. 13D).

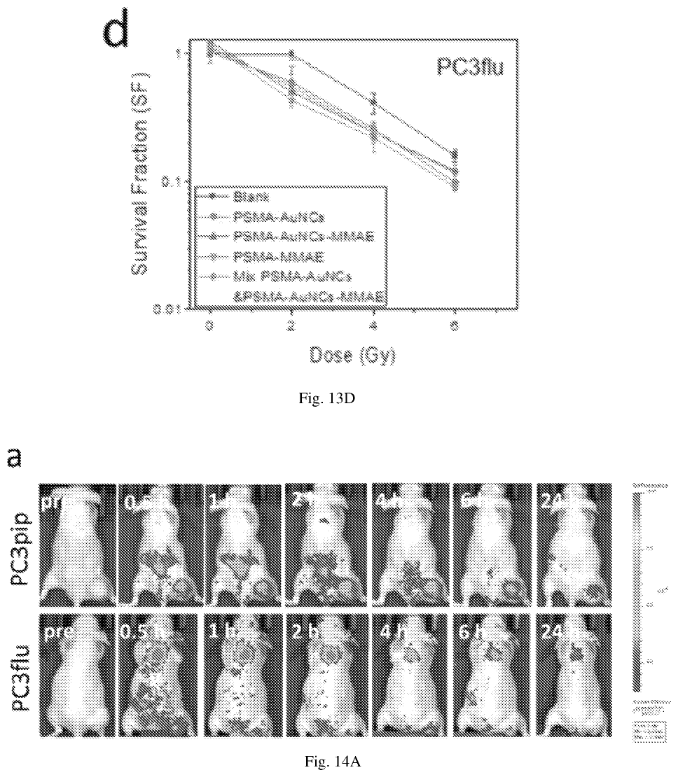

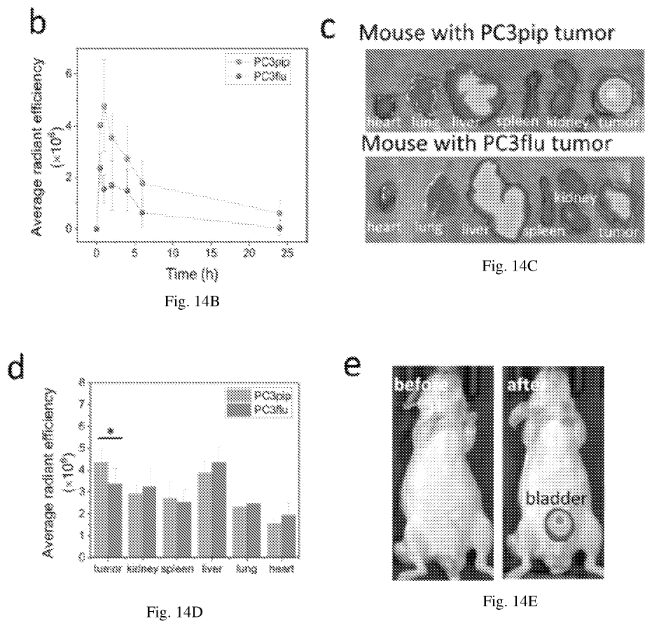

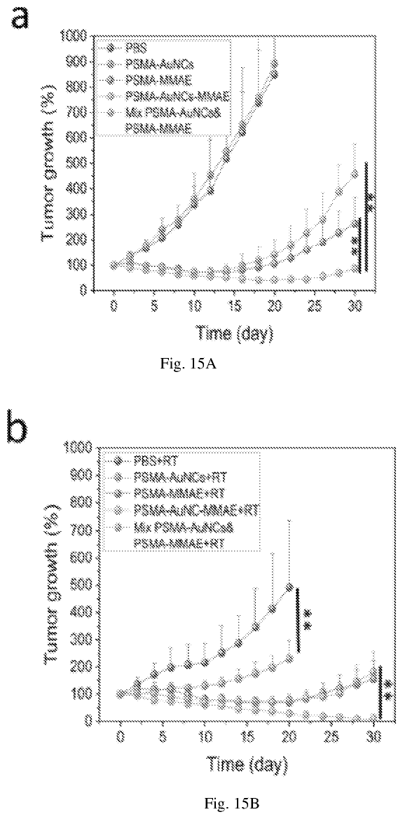

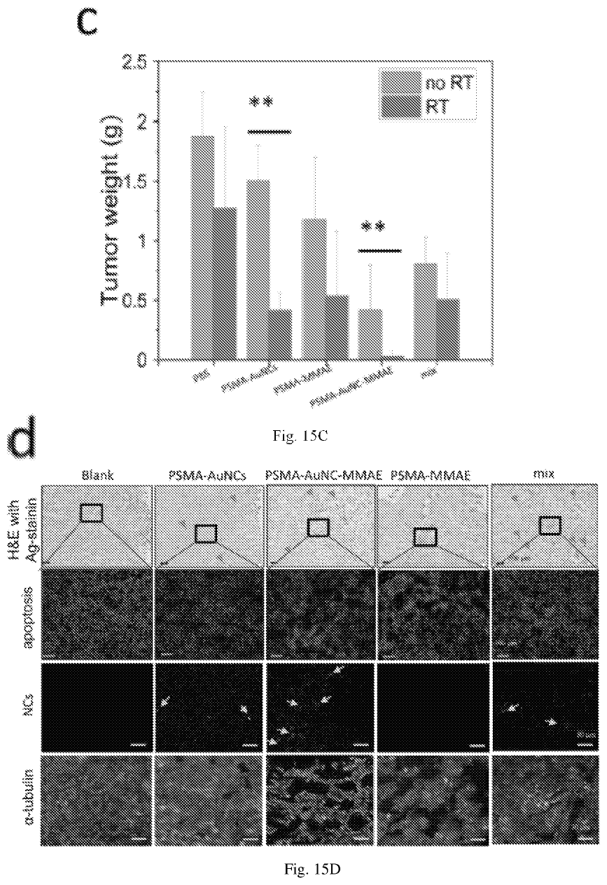

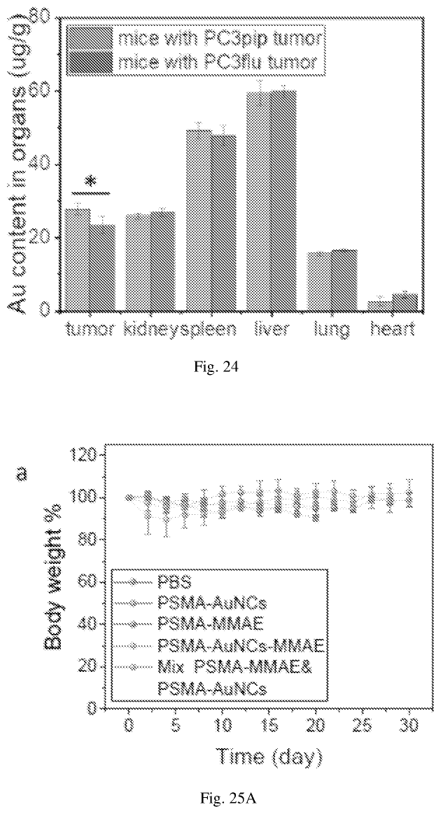

To investigate the selective tumor uptake and distribution in vivo, we injected the PSMA-AuNCs to the PC3pip or PC3flu tumor bearing mice at 0.25 g/kg and monitored the fluorescence of NCs with the Spectrum IVIS imager before injection and at 0.5, 1, 2, 4, 6, and 24 h postinjection. There was a strong fluorescence from the PC3pip tumor as early as 0.5 h postinjection that reached maximum at 1 h, after which the signal decayed over time (FIG. 14A). In contrast, mice bearing PC3flu tumors had much less fluorescence from the tumors. The fluorescence intensity is a direct reflection of NCs accumulation in tumors. Average radiant efficiency for PC3pip tumors at 1 h was 4.8×10⁶ (photons s⁻¹ cm⁻² sr⁻¹ (μW cm⁻²)⁻¹), which was around 3 times that for PC3flu tumors, 1.7×10⁶ (photons s⁻¹ cm⁻² sr⁻¹ (μW cm⁻²)⁻¹) (FIG. 15B). After 24 h, all the mice were euthanized, and NCs distribution in the main organs was imaged ex vivo, showing the brightest signal in the liver and greater fluorescence in PC3pip than PC3flu tumors (FIG. 15C,D). Similar distribution results were confirmed by ICP measurement (FIG. 24). Significantly higher NCs retention in PC3pip than in PC3flu tumor at 24 h postinjection was confirmed by both NCs fluorescence and ICP analysis. This was due to increased uptake of PSMA-targeted NCs by PC3pip tumors resulting from their overexpression of PSMA receptor. Compared to gold nanoparticles of a larger size, which have a dramatically higher retention in liver and spleen, the PSMA-AuNCs showed minimal retention in all organs (FIG. 15D). The difference in vivo kinetics was likely due to the extremely small size of NCs that enabled them to escape from the reticuloendothelial system and be renally excreted. This was evidenced by the observation of significantly high fluorescence signal from the bladders after injection of PSMA-AuNCs (FIG. 15E).

We next evaluated the antitumor activity of PSMA-AuNC-MMAE conjugates in mice bearing flank PC3pip tumors. The PSMA-AuNC-MMAE were injected, and a radiation dose of 6 Gy was given 4 h postinjection, focused on the tumor area only. For the no radiation control groups (FIG. 15A), PSMA-AuNCs injected mice showed similar tumor growth kinetics to the PBS control group, indicating that PSMA-AuNCs alone did not affect the tumor growth. The PSMA-MMAE group showed significant tumor growth inhibition with tumor volume decreasing over the first 2 weeks but then slowly increasing in size with an increase of 163% at the end of observation. The PSMA-AuNC-MMAE showed a much better tumor inhibition than PSMA-MMAE, with tumor growth coming back after 3 weeks but tumor volume still reduced by 23% at day 30. With radiation (FIG. 15B), obvious radiosensitization by Au was observed with a tumor volume increase of only 130% compared to 390% for the PBS/RT group at day 20. The best therapeutic outcome was observed for the PSMA-AuNC-MMAE group, having an average tumor volume decrease of 90.2% at day 30, with two mice in this group completely cured. For the PSMA-MMAE group, tumor volume increased 55% at day 30. The body weight for all the groups or mice with and without RT did not show overt fluctuation. The PSMA-AuNC-MMAE injected mice showed some body weight reduction initially, but they all recovered after 1 week (FIG. 25). This may be induced by PSMA-AuNC-MMAE retention in organs and nonspecific MMAE release. To test this, we added a group of mice treated with a mixture of PSMA-AuNCs and PSMA-MMAE having equivalent Au and MMAE, and the mice did not experience body weight reduction. Interestingly, lower therapeutic efficacy was observed for this mixture group compared to the PSMA-AuNC-MMAE conjugates. This may be due to the competition effect of PSMA-targeted small molecules and PSMA-targeted NCs. The tumor weight for each of the groups at the end of observation indicated that RT reduced the tumor growth for each group overall, but PSMA-AuNCs and PSMA-AuNC-MMAE groups showed significant changes, indicating the excellent radiosensitization imparted by gold (FIG. 15C).

To further compare the damage induced by MMAE, tumors for the five groups without radiation were retrieved and analyzed histologically (FIG. 15D). H&E and immunohistochemistry staining demonstrated that all the PSMA-MMAE, PSMA-AuNC-MMAE, and the mixed conjugate groups showed a substantial degree of apoptosis, but the PSMA-AuNC-MMAE group showed the most dramatic tumor tissue damage. A silver staining assay for the NCs also revealed the distribution of the nanoclusters within the tumor tissues (black dots as indicated by red arrows), which was confirmed by the fluorescence imaging of the tumor slices with the presence of NCs fluorescence (FIG. 15D, arrows in third panel). Immunochemistry staining of activated caspase-3 also revealed that apoptosis of tumor tissues induced by MMAE had the greatest fluorescence, indicating significant apoptosis for the PSMA-AuNC-MMAE group. Because the upregulated cathepsin activity in prostate tumors should cleave the MMAE from either PSMA-MMAE or PSMA-AuNC-MMAE and result in u-tubulin disruption, the u-tubulin staining for the tumor samples was also carried out. Both the PBS and PSMA-AuNCs treated group displayed dense fibrous u-tubulin networks. In contrast, the PSMA-MMAE, PSMA-AuNC-MMAE, or the mixture groups showed disruption of the u-tubulin network exemplified as bright green dots in the tumor tissues, which was also observed in vitro when the tumor cells were dying.

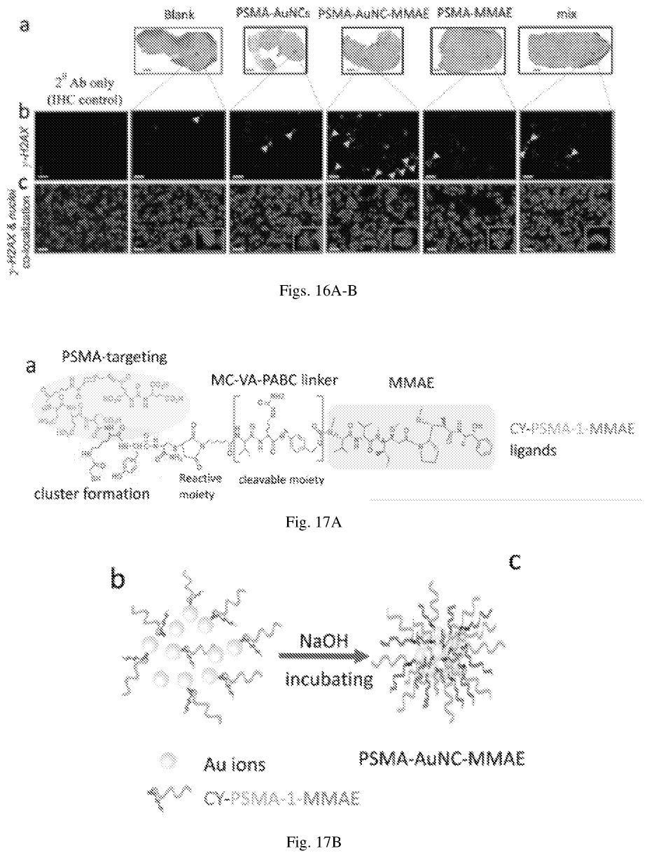

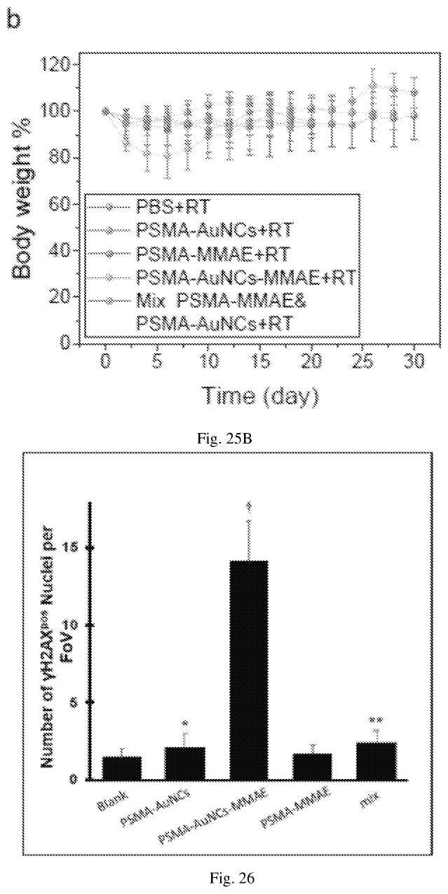

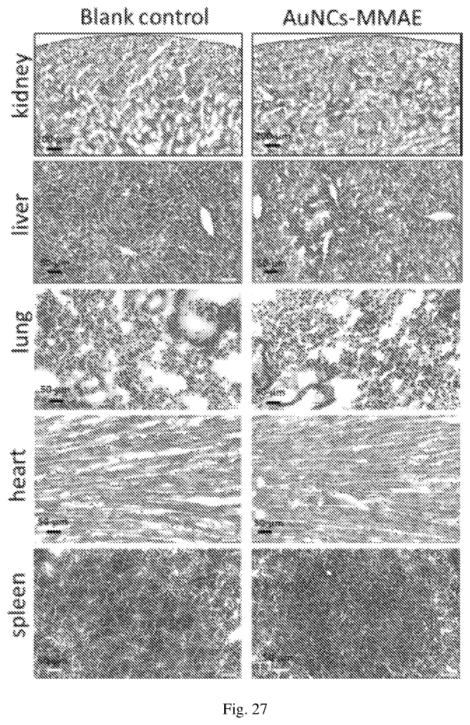

The radiation-induced DNA double-strand breaks in tumors and sensitizing enhancement from different groups were evaluated by y-H2AX staining (FIG. 16). After irradiation, massive DNA damage was observed for the PSMA-AuNC-MMAE group as shown by red fluorescence (arrows) colocalized with the nuclei. For PSMA-AuNCs alone, PSMA-MMAE, and the mixture groups, there was also a large amount of DNA damage, but much less compared to the PSMA-AuNC-MMAE group. Statistically, tumors treated with PSMA-AuNC-MMAE showed a significantly higher (p<0.0001) y-H2AX staining of nuclei than observed with the other groups (FIG. 26). Taken together, these results clearly validate and confirm a synergistic therapeutic effect by both Au and MMAE molecules (coefficient of drug interaction, CDI=0.9).

For successful clinical translation any possible side effect and toxicity related to RT and chemotherapy should be minimized. Advances in imaging technologies enable precise delivery of radiation doses to defined tumor areas, and radiation amplification can be achieved with radiosensitizers. For chemotherapy, the highly toxic therapeutic drugs can also be modified to improve specificity via tumor biomarker recognition. We developed a PSMA-AuNC-MMAE conjugate with MMAE conjugated via a self-immolative cathepsin cleavable linker. The PSMA-AuNC-MMAE conjugates showed excellent selectivity for PSMA-expressing tumor cells and antitumor activity upon cathepsin cleavage to release free MMAE. The prodrug approach, selective accumulation of PSMA-AuNC-MMAE in tumors, ensuing radiosensitization, and the conjugate's rapid urinary excretion may possibly reduce the toxicity to animals induced by either radiation or nonspecific release of MMAE. Biodistribution studies with PSMA-AuNC, which has an average size smaller than the threshold of renal clearance limit (around 5.5 nm), may result in a fast renal excretion and a reduced circulation time in vivo, which is beneficial for reducing the toxicity. According to the biodistribution, the PSMA-AuNCs peaked in tumor tissues around 1 h post injection and showed a low retention in the reticuloendothelial system (RES) after 24 h, which is in agreement with other AuNCs studies. In contrast, the majority AuNPs having a much larger size end up in RES organs.

On the other hand, the choice of a linker to conjugate chemotherapy drugs to targeting ligands also plays a critical role for their successful application. MMAE conjugated to antibody via uncleavable linkers is inactive until completely released via lysosomal proteolytic degradation of the antibody. A cathepsin self-imolative cleavable linker, MC-VC-PABC, was used here and showed controllable MMAE release upon cathepsin activity. This linker rendered the MMAE inactive until protease removal and liberation of free MMAE. This approach could take full advantage of upregulated cathepsin activities in tumor tissues and further reduce the toxicity of MMAE. Although antibodies conjugated with MMAE via the VC linker have been reported to exhibit instability in plasma, this may be related to the significantly extended half-life of ADCs in the blood. Our approach uses a much smaller carrier that is rapidly excreted from the body limiting nonselective release of MMAE. The PSMA-AuNC-MMAE showed good antitumor activity; however, they still caused some body weight loss the first 6 days after injection, which may also be related to the unavoidable nontumor selective cleavage of MMAE from PSMA-AuNC-MMAE during circulation. However, no obvious damage to the main organs was observed for the PSMA-AuNC-MMAE treated mice at the end of treatment (FIG. 17). Other studies have used external trigger-responsive linkers to conjugate antitumor drugs to reduce toxicity caused by nonselective release. Generally in clinical uses of antitumoral drugs, multiple doses at intervals will be utilized. Additionally, radiation doses are also given in multiple fractionations. Therefore, the injected PSMA-AuNC-MMAE for each dose may be lower, further reducing toxicity. To better control the release of MMAE from NCs and minimize toxicity, other responsive linkers and the mode of PSMA-AuNC-MMAE administration will be investigated in our future studies.

A combined chemotherapy and RT has been prescribed to a wide range of patients with cancer. The PSMA-AuNC-MMAE conjugates integrated with both gold and MMAE enables precision chemotherapy-RT. Interestingly, without radiation, the PSMA-AuNC-MMAE showed better antitumor capability than the PSMA-MMAE counterpart, which was probably due to the enhanced binding to PSMA for nanoparticles and increased drug delivery by the NCs. With a single dose of 6 Gy radiation, we also observed a synergistic sensitizing effect by both gold and MMAE in PSMA-AuNC-MMAE (CDI=0.9). However, compared to PSMA-MMAE, PSMA-AuNCs had a much better sensitizing enhancement as measured by γ-H2AX staining for tumor tissues. The independent mechanism of the two therapies will also decrease the chance of tumor cells developing resistance. Moreover, X-ray irradiation may further enhance the accumulation of NCs in tumor tissues via disruption of the tumor vasculature. Though the retrovirally modified PSMA-expressing PC3 cells are a good model to evaluate the PSMA-targeted NCs, AR positive prostate cancer models that express PSMA, such as VCaP, C4-2b, and PDX models will be used in the future to evaluate the performance of NCs in different types of prostate cancers. We are hopeful that the future studies will move us closer to clinical translation of the PSMA-targeted nanoclusters.

In summary, we synthesized a PSMA-AuNC-MMAE conjugates for combined RT and chemotherapy of PCa. The NCs were functionalized with PSMA-1 targeting moieties and conjugated with MMAE via a cleavable linker, which demonstrated selective tumor cell uptake and active MMAE release upon cathepsin cleavage in vitro and in vivo. RT was enhanced by both gold and MMAE, and together with the chemotherapeutic effect of MMAE, the PSMA-AuNC-MMAE displayed excellent antitumor activity. Further studies for optimizing the dose and administration schedule for minimizing systemic toxicity will be performed to help translate the PSMA-AuNC-MMAE conjugates for clinical applications.

### Example 4

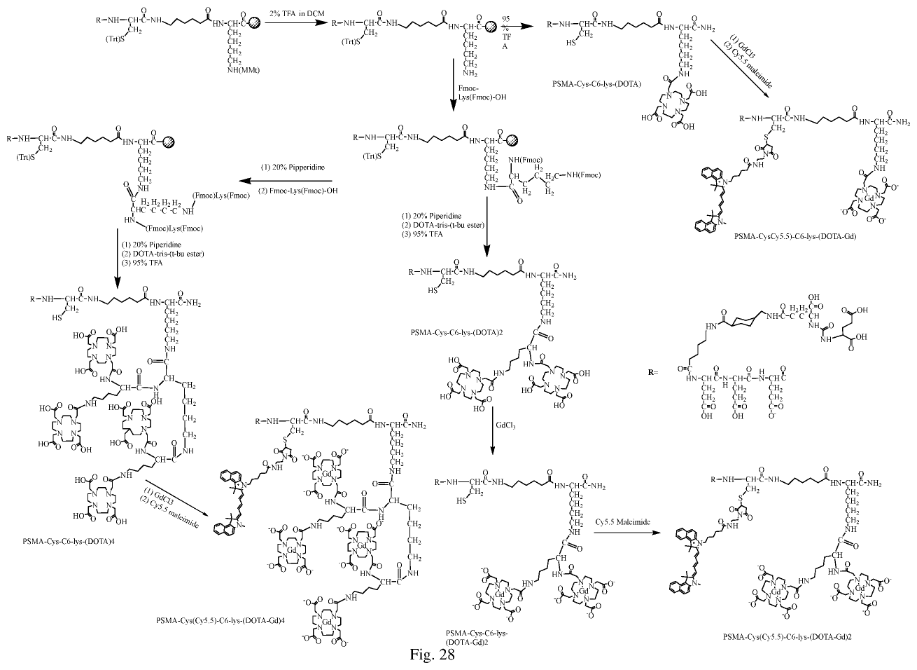

Magnetic resonance (MR) imaging is a clinically relevant, noninvasive diagnostic tool for high-resolution anatomic and functional imaging. Molecular MR imaging enables the visualization of biological markers in vivo. Gd-based contrast agents are widely accepted by clinicians because they are easy to administer and provide T1-weighted, positive contrast. With signal amplification strategies, MR might offer a sensitive modality for molecular imaging complementary to radionuclide-based techniques. Combining a receptor-specific high affinity ligand with multimeric Gd agents for detection has been described as one solution for enabling MR-based receptor imaging. PSMA is overexpressed in primary and metastatic prostate cancer, particularly with respect to the castration resistant form. Furthermore, PSMA is expressed by most solid tumors and tumor neovasculature. We hypothesized that PSMA would be a suitable biomarker for MR molecular imaging. We plan to synthesize three PSMA-targeted MR contrast agent with 1 (PSMA-Cys(Cy5.5)-C6-Lys-(DOTA-Gd)), 2 (PSMA-Cys(Cy5.5)-C6-Lys-(DOTA-Gd)2) and 4 Gd (PSMA-Cys(Cy5.5)-C6-Lys-(DOTA-Gd)4) (FIG. 28). To adjust the pharmacokinetics of the contrast agents, fluorescent dye Cy5.5 will be added to the molecules.

### Synthesis of PSMA-1-Cys(Trt)-C6-Lys(NH2)-MBHA Rink-Amide Resin

PSMA-1-Cys(Trt)-C6-Lys(Mmt) on MBHA Rink-amide resin was synthesized manually based on Fmoc chemistry using the same method as the synthesis of PSMA-1. After all the sequence was assembled on Rink-Amide resin, the resin was exposed to 2% TFA in dichloromethane to selectively remove the Mmt group to get PSMA-1-Cys(Trt)-C6-Lys(NH2)-MBHA Rink-amide resin.

### Synthesis of PSMA-1-Cys(Cy5.5)-C6-Lys(DOTA-Gd)

To PSMA-1-Cys(Trt)-C6-Lys(NH2)-MBHA Rink-amide resin in DMF, 3.3 equiv of DOTA-tris-(t-Bu ester) in DMF activated with 3.3 equiv of HCTU and 5 equiv of diisopropylethylamine (DIPEA) was added. After the reaction was complete, the resin was washed with DMF and then dichloromethane and dried. The peptide was cleaved from resin by TFA/water/triisopropylsilane (950:25:25). The cleaved peptide PSMA-1-Cys-C6-Lys(DOTA) was purified by preparative HPLC and lyophilized. The lyophilized PSMA-1-Cys-C6-Lys(DOTA) was dissolved in water, to which 5-fold excess amount of GdCl₃ in water was added. The reaction mixture was stirred at room temperature for 1 day and then purified by HPLC to get pure PSMA-1-Cys-C6-Lys(DOTA-Gd). Finally, PSMA-1-Cys-C6-Lys(DOTA-Gd) in PBS solution was reaction with 1.5-fold excess amount of Cy5.5 maleimide for 2 hours at room temperature. The crude was then purified by HPLC to get PSMA-1-Cys(Cy5.5)-C6-Lys(DOTA-Gd). (The compounds in this series haven't been synthesized yet).

### Synthesis of PSMA-1-Cys(Cy5.5)-C6-Lys(DOTA-Gd)2

To PSMA-1-Cys(Trt)-C6-Lys(NH2)-MBHA Rink-amide resin in DMF, 3.3 equiv of Fmoc-Lys(Fmoc)-OH activated with 3.3 equiv of HCTU and 5 equiv of diisopropylethylamine (DIPEA) was added. After the reaction was completed, the resin was washed 5 times. The two Fmoc groups of N-terminal amino acid lysine were deprotected by 20% piperidine in DMF. The resin was washed 5 times with DMF, to which 3.3 equiv of DOTA-tris-(t-Bu ester) in DMF activated with 3.3 equiv of HCTU and 5 equiv of diisopropylethylamine (DIPEA) in DMF was added. After the reaction was complete, the resin was washed with DMF and then dichloromethane and dried. The peptide was cleaved from resin by TFA/water/triisopropylsilane (950:25:25). The cleaved peptide PSMA-1-Cys-C6-Lys(DOTA)2 was purified by preparative HPLC and lyophilized. MALDI-MS: C₉₃H₁₅₃N₂₂O₃₇S, 2204.1 (found); 2204.3 (calculated). PSMA-1-Cys-C6-Lys(DOTA)2 was then reacted with 5-fold excess amount of GdCl₃ in water for 1 day. The crude was purified by HPLC to get pure PSMA-1-Cys-C6-Lys(DOTA-Gd)2. MALDI-MS: C₉₃H₁₄₇Gd₂N₂₂O₃₇S, 2511.8 (found); 2511.8 (calculated). PSMA-1-Cys-C6-Lys(DOTA-Gd)2 in PBS solution was reaction with 1.5-fold excess amount of Cy5.5 maleimide for 2 hours at room temperature. The crude was then purified by HPLC to get PSMA-1-Cys(Cy5.5)-C6-Lys(DOTA-Gd)2. MALDI-MS: C₁₃₉H₁₉₆Gd₂N₂₆O₄₀S, 3217.1 (found); 3217.2 (calculated).

### Synthesis of PSMA-1-Cys(Cy5.5)-C6-Lys(DOTA-Gd)4

To PSMA-1-Cys(Trt)-C6-Lys(NH2)-MBHA Rink-amide resin in DMF, 3.3 equiv of Fmoc-Lys(Fmoc)-OH activated with 3.3 equiv of HCTU and 5 equiv of diisopropylethylamine (DIPEA) in DMF was added. After the reaction was completed, the resin was washed 5 times. The two Fmoc groups of N-terminal amino acid lysine were deprotected by 20% piperidine in DMF. The resin was washed 5 times with DMF, then 3.3 equiv of Fmoc-Lys(Fmoc)-OH activated with 3.3 equiv of HCTU and 5 equiv of diisopropylethylamine (DIPEA) in DMF was added. After the reaction was completed, the four Fmoc groups were removed by 20% piperidine. The resin was washed 5 times with DMF, to which 3.3 equiv of DOTA-tris-(t-Bu ester) in DMF activated with 3.3 equiv of HCTU and 5 equiv of diisopropylethylamine (DIPEA) in DMF was added. After the reaction was complete, the resin was washed with DMF and then dichloromethane and dried. The peptide was cleaved from resin by TFA/water/triisopropylsilane (950:25:25). The cleaved peptide PSMA-1-Cys-C6-Lys(DOTA)4 was purified by preparative HPLC and lyophilized. MALDI-MS: C₁₃₇H₂₃₀N₃₄O₅₃S, 3232.5 (found); 3232.5 (calculated). PSMA-1-Cys-C6-Lys(DOTA)4 was then reacted 5-fold excess amount of GdCl₃ in water for 1 day. The crude was purified by HPLC to get pure PSMA-1-Cys-C6-Lys(DOTA-Gd)4. MALDI-MS: C₁₃₇H₂₁₈Gd₄N₃₄O₅₃S, 3851.2 (found); 3851.2 (calculated). PSMA-1-Cys-C6-Lys(DOTA-Gd)4 in PBS solution was reaction with 1.5-fold excess amount of Cy5.5 maleimide for 2 hours at room temperature. The crude was then purified by HPLC to get PSMA-1-Cys(Cy5.5)-C6-Lys(DOTA-Gd)4. MALDI-MS: C₁₈₃H₂₆₇Gd₄N₃₈O₅₆S, 3217.1 (found); 4556.6 (calculated).

### In Vivo Imaging

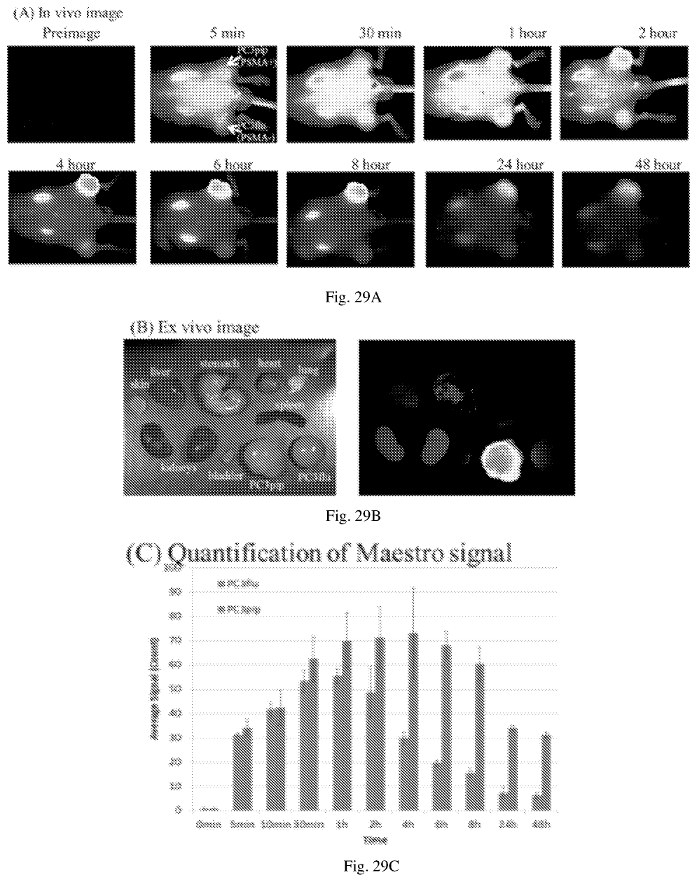

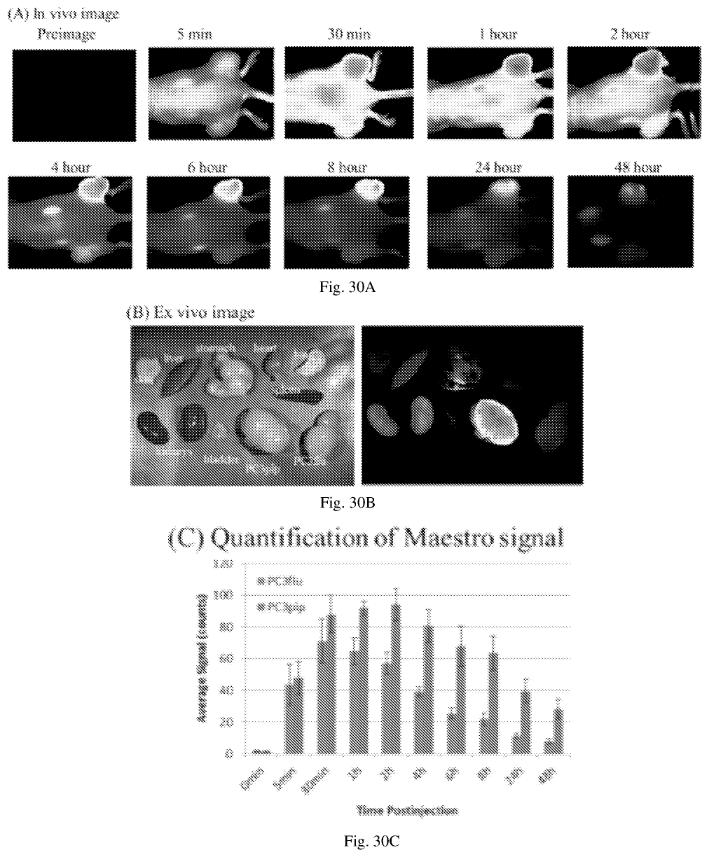

For in vivo experiments, we first tested if the new probes can selectively bind to PSMA expressing PC3pip tumors using Maestro In Vivo imaging system. Mice bearing PSMA-positive PC3pip and PSMA-negative PC3flu tumors received 1 nmol of PSMA-1-Cys(Cy5.5)-C6-Lys(DOTA-Gd)2 through tail vein injection; images were taken at different time points post injection. At 2 hours post injection, selective accumulation in PC3pip was observed and the signal reached highest at 4 hours post injection, then cleared out from the body gradually (FIG. 29). The signal in PC3pip tumor was 2 fold higher than that in PC3flu signal at 4 hours post injection. The difference between PC3pip and PC3flu tumor was even bigger at later time points. Ex vivo imaging of organs at 48 hours post injection showed that the signal was mainly in the PC3pip tumor, other organs including kidneys showed minimal fluorescence. Similar results were observed when mice received PSMA-1-Cys(Cy5.5)-C6-Lys(DOTA-Gd)4 with selective uptake in PC3pip tumors (FIG. 30).

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A compound comprising the formula (I): 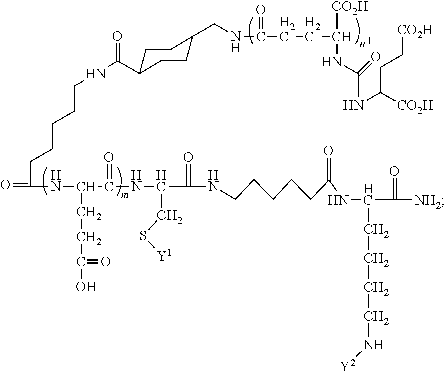
wherein:
n¹ is 1, 2, 3, or 4;
Y¹ and Y² each independently include at least one detectable moiety, therapeutic agent, or a theranostic agent.

2: The compound of claim 1, wherein Y¹ and Y² include at least one radiolabel or radioisotope, radiosensitizer, chelating agent, photosensitizer, fluorescent labeling agent, magnetic resonance imaging (MRI) agent, or anti-cancer agent.

3-11. (canceled)

12: The compound of claim 2, wherein the anticancer agent is linked to the thio group or amine group via a protease cleavable or acid-labile linker.

13: The compound of claim 12, wherein the linker includes a maleimido-caproyl linker-valine-citrulline cleavable peptide-p-aminobenzyl carbamate spacer (MC-VC-PABC) protease cleavable linker.

14: The compound of any of claim 1, wherein at least one of Y¹ or Y² includes an anti-cancer agent and the other Y¹ or Y² includes a photosensitizer, radiosensitizer, or fluorescent imaging agent.

15: The compound of claim 1 selected from the following: 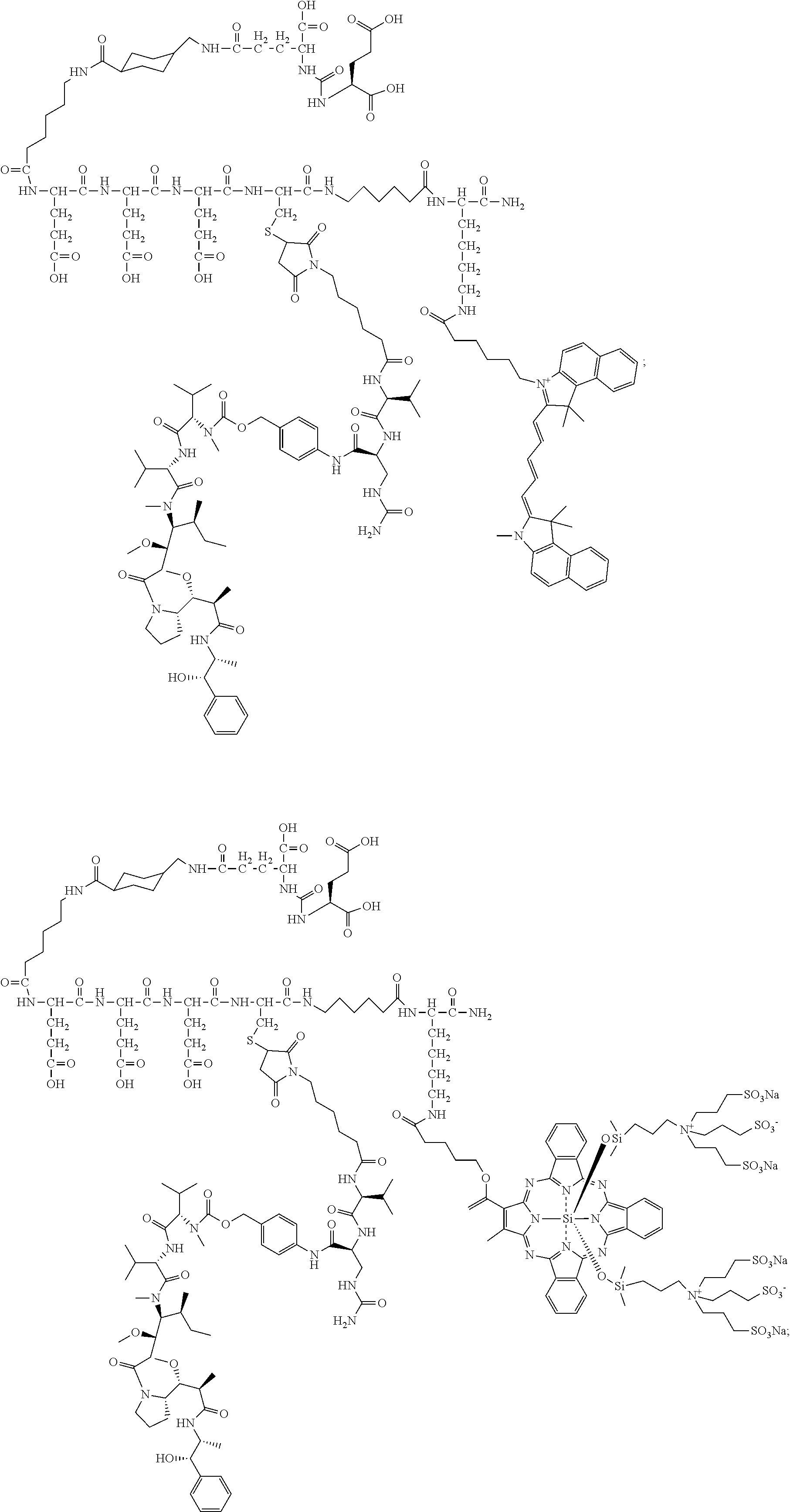 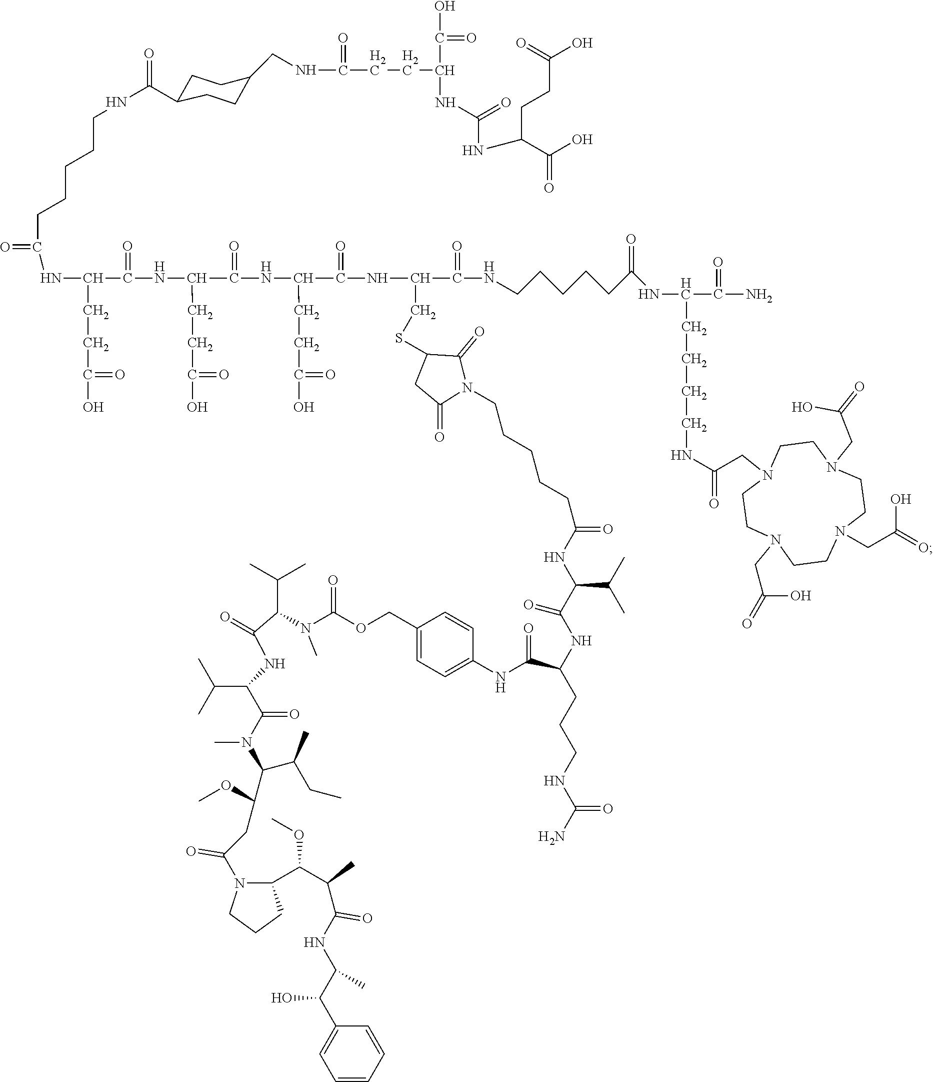 [SMILES: CC[C@H](C)[C@@H]([C@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SCC(NC(=O)C(CCC(=O)O)NC(=O)C(CCC(=O)O)NC(=O)C(CCC(=O)O)NC(=O)CCCCCNC(=O)C3CCC(CNC(=O)CCC(NC(=O)NC(CCC(=O)O)C(=O)O)C(=O)O)CC3)C(=O)NCCCCCC(=O)NC(CCCCNC(=O)CN3CCN(CC(=O)O)CCN(CC(=O)O)CCN(CC(=O)O)CC3)C(N)=O)C2=O)C(C)C)cc1)C(C)C]
or a pharmaceutically acceptable salt thereof.

16: A compound comprising the following formula: 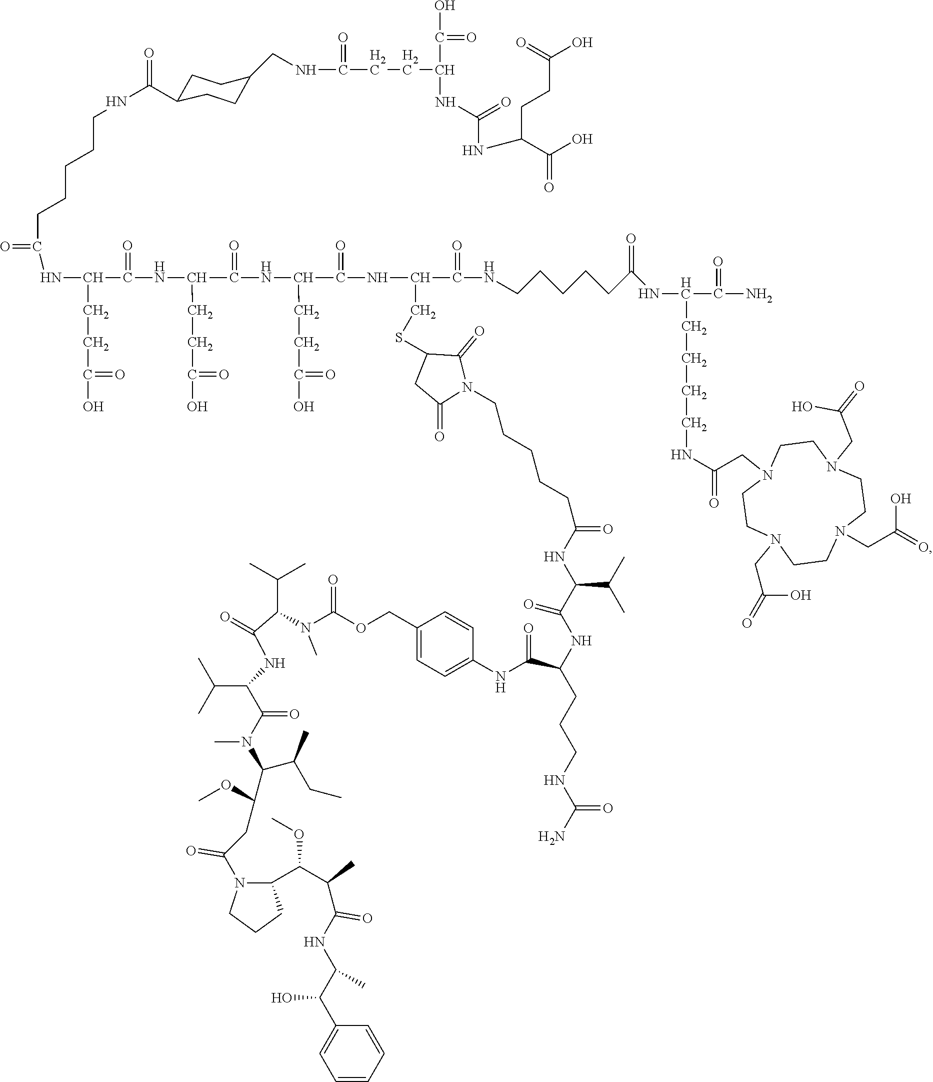 [SMILES: CC[C@H](C)[C@@H]([C@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SCC(NC(=O)C(CCC(=O)O)NC(=O)C(CCC(=O)O)NC(=O)C(CCC(=O)O)NC(=O)CCCCCNC(=O)C3CCC(CNC(=O)CCC(NC(=O)NC(CCC(=O)O)C(=O)O)C(=O)O)CC3)C(=O)NCCCCCC(=O)NC(CCCCNC(=O)CN3CCN(CC(=O)O)CCN(CC(=O)O)CCN(CC(=O)O)CC3)C(N)=O)C2=O)C(C)C)cc1)C(C)C]
optionally complexed with a metal, or a pharmaceutically acceptable salt thereof.

17: The compound of claim 16, wherein the metal ion includes at least one of ¹⁸⁶Re, ¹⁸⁸Re, ⁹⁹mTc, ¹⁵³Gd, ¹¹¹In, ⁶⁷Ga, ⁶⁸Ga, ²⁰¹TI, ⁸²Rb, ⁶⁴Cu, ⁸⁹Zr, ⁹⁰Y, ¹⁷⁷Lu, T(tritium), ¹⁴⁹Tb, ¹⁶¹Tb, ¹⁵³Sm, ⁸⁹Sr, ²¹¹At, ²²⁵Ac, ²²⁷Ac, ¹²³⁻¹²⁵I, ¹³¹I, ⁶⁷Cu, ²⁰³Pb, ²¹²Pb, ²¹¹Bi, ²¹³Bi, or ²²⁷⁻²³³Th.

18: The compound of claim 1, not binding to aquaporin.

19: The compound of claim 1 having a selectivity for PSMA expressing cancer tissue versus non-PSMA expressing non-cancer tissue ≥5 times.

20: The compound of claim 1, upon administration to a subject in need thereof showing minimal accumulation or uptake in salivary glands of the subject.
